(19) United States
(12) Patent Application Publication
(10) Pub. No.: US 2023/0061456 A1
(43) Pub. Date: Mar. 2, 2023

(54) METHODS AND COMPOSITIONS FOR GENERATING TYPE 1 VESTIBULAR HAIR CELLS

(71) Applicant: Decibel Therapeutics, Inc., Boston, MA (US)

(72) Inventors: Joseph BURNS, Newton, MA (US); Noah DRUCKENBROD, Dedham, MA (US); Tyler GIBSON, Boston, MA (US); Ryan MCCARTHY, Ashland, MA (US); Jonathan B. SELLON, Boston, MA (US); Kathy SO, Boston, MA (US)

(21) Appl. No.: 17/794,673

(22) PCT Filed: Jan. 22, 2021
(86) PCT No.: PCT/US2021/014788

§ 371 Date: Jul. 22, 2022

Related U.S. Application Data
(60) Provisional application No. 62/965,783, filed on Jan. 24, 2020.

(51) Int. Cl. C12N 15/113; C12N 15/86; A61K 38/17; A61K 31/713

(52) U.S. Cl. CPC C12N 15/1136; C12N 15/86; A61K 38/1709; A61K 31/713; C12N 2310/141; C12N 2310/531; C12N 2750/14143; C12N 2320/31

(57) ABSTRACT

The disclosure provides Sox2 inhibitors that can be used to generate Type I vestibular hair cells in the vestibular system. The Sox2 inhibitors may be administered to a subject alone or in combination with a regeneration agent to convert Type II vestibular hair cells or regenerated vestibular hair cells to Type I vestibular hair cells.

### SEQUENCE LISTING

The instant application contains a Sequence Listing which has been submitted electronically in ASCII format and is hereby incorporated by reference in its entirety. The ASCII copy, created on Jan. 22, 2021, is named 51124-079WO2_Sequence_Listing_1_22_21_ST25 and is 63,389 bytes in size.

### BACKGROUND

Vestibular dysfunction is a major public health issue that has profound consequences on quality of life. Approximately 35% of US adults age 40 years and older exhibit balance disorders and this proportion dramatically increases with age, leading to disruption of daily activities, decline in mood and cognition, and an increased prevalence of falls among the elderly. Vestibular dysfunction is often acquired, and has a variety of causes, including disease or infection, head trauma, ototoxic drugs, and aging. A common factor in the etiology of vestibular dysfunction is the damage to vestibular hair cells of the inner ear.

Vestibular hair cells detect forces applied to the head, allowing for a sense of position in space as well as compensatory postural and eye movements required for balance. Vestibular hair cells degenerate with age, and they can be destroyed by therapeutic drugs such as aminoglycoside antibiotics. Extensive loss of vestibular sensory cells is highly debilitating and can elicit nauseating bouts of dizziness, imbalance, and incapacitation. Vestibular deficits are prevalent in the human population. They are estimated to affect 35% of the U.S. population >40 years old, and they increase significantly with age (Burns and Stone, 2017. Semin. Cell Dev. Biol. 65:96-105).

Thus, there is still a need to develop therapies aimed at restoring hair cell function that would be beneficial to patients suffering from vestibular dysfunction.

### SUMMARY OF THE INVENTION

The present invention provides compositions and methods for treating vestibular dysfunction (e.g., vertigo, dizziness, balance loss, bilateral vestibulopathy (bilateral vestibular hypofunction), oscillopsia, or a balance disorder) in a subject, such as a human subject. The compositions and methods of the disclosure pertain to Sox2 inhibitors that can be delivered to a Type II vestibular hair cell, a regenerated hair cell, or a supporting cell to reduce Sox2 expression or activity, leading to the generation of Type I vestibular hair cells. The Sox2 inhibitors can also be administered in combination with a regeneration agent, such as an agent that increases Atoh1 expression or an agent that reduces Notch expression or activity.

In a first aspect, the invention provides a method of generating Type I vestibular hair cells in a human subject in need thereof by administering to the subject an effective amount of a Sox2 inhibitor. In some embodiments, the subject has or is at risk of developing vestibular dysfunction.

In another aspect, the invention provides a method of treating a subject having or at risk of developing vestibular dysfunction by administering to the subject an effective amount of a Sox2 inhibitor.

In another aspect, the invention provides a nucleic acid vector comprising a Sox2 inhibitor operably linked to a promoter.

In another aspect, the invention provides an shRNA molecule containing a nucleotide sequence that has at least 70% complementarity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity) complementarity to any one of SEQ ID NOs: 11, 25, 26, 27, 28, and 29. In some embodiments, the shRNA molecule contains a nucleotide sequence that has 100% complementarity to any one of SEQ ID NOs: 11, 24, 26, 27, 28, and 29.

In another aspect, the invention provides an shRNA molecule containing a sequence of nucleotides 2234-2296 of SEQ ID NO:30 or nucleotides 2234-2296 of SEQ ID NO:32.

In some embodiments of any of the foregoing aspects, the shRNA is embedded in a microRNA (miRNA) backbone. In some embodiments, the miRNA backbone is a miR-30 or mir-E backbone. In some embodiments, the shRNA includes a sequence of nucleotides 2109-2426 of SEQ ID NO: 30, nucleotides 2109-2408 of SEQ ID NO: 31, nucleotides 2109-2426 of SEQ ID NO: 32, or nucleotides 2109-2408 of SEQ ID NO: 33.

In another aspect, the invention provides an siRNA including a sense strand and an antisense strand and selected from the following pairs: SEQ ID NO: 35 and SEQ ID NO: 36; SEQ ID NO: 37 and SEQ ID NO: 38; SEQ ID NO: 39 and SEQ ID NO: 40; and SEQ ID NO: 41 and SEQ ID NO: 42.

In some embodiments of any of the foregoing aspects, the Sox2 inhibitor is an inhibitory RNA molecule targeting Sox2 or a Sox2 promoter, a component of a gene editing system targeting Sox2, a dominant negative Sox2 protein, or polynucleotide encoding a dominant negative Sox2 protein. In some embodiments of any of the foregoing aspects, the Sox2 inhibitor is an inhibitory RNA molecule targeting Sox2 (e.g., Sox2 mRNA, such as human Sox2 mRNA (SEQ ID NO: 1) or murine Sox2 (SEQ ID NO: 3)). In some embodiments, the inhibitory RNA molecule is a short interfering RNA (siRNA). In some embodiments, the inhibitory RNA molecule is a short hairpin RNA (shRNA). In some embodiments, the siRNA or shRNA targeting Sox2 has a nucleobase sequence containing a portion of at least 8 contiguous nucleobases (e.g., 8, 9, 10, 11, 12, 13, 14, 15, 16, 17, 18, 19, 20, or more nucleobases) having at least 70% complementarity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity) to an equal length portion of a target region of an mRNA transcript of a human (e.g., SEQ ID NO: 1) or murine (SEQ ID NO: 3) SOX2 gene. In some embodiments the target region is at least 8 to 21 (e.g., 8 to 21, 9 to 21, 10 to 21, 11 to 21, 12 to 21, 13 to 21, 14 to 21, 15 to 21, 16 to 21, 17 to 21, 18 to 21, 19 to 21, 20 to 21, or all 21) contiguous nucleobases of any one of SEQ ID NOs: 5-23. In some embodiments the target region is at least 8 to 19 (e.g., 8 to 19, 9 to 19, 10 to 19, 11 to 19, 12 to 19, 13 to 19, 14 to 19, 15 to 19, 16 to 19, 17 to 19, 18 to 19, or all 19) contiguous nucleobases of any one of SEQ ID NOs: 25-27. In some embodiments the target region is at least 8 to 22 (e.g., 8 to 22, 9 to 22, 10 to 22, 11 to 22, 12 to 22, 13 to 22, 14 to 22, 15 to 22, 16 to 22, 17 to 22, 18 to 22, 19 to 22, 20 to 22, 21 to 22, or all 22) contiguous nucleobases of SEQ ID NO: 28 or SEQ ID NO: 29. In some embodiments, the shRNA has a nucleobase sequence having at least 70% complementarity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity) complementarity to any one of SEQ ID NO:11, SEQ ID NO: 25, SEQ ID NO: 26, SEQ ID NO: 27, SEQ ID NO: 28, and SEQ ID NO: 29. In some embodiments, the shRNA contains the sequence of nucleotides 2234-2296 of SEQ ID NO: 30 or nucleotides 2234-2296 of SEQ ID NO: 32. In some embodiments, the shRNA is embedded in a micro RNA (miRNA; e.g., embedded in an miRNA backbone to produce an shRNA-mir). In some embodiments, the shRNA is embedded in a miR-30 backbone. In some embodiments, the shRNA is embedded in mir-E backbone. In some embodiments, the shRNA contains the sequence of nucleotides 2109-2426 of SEQ ID NO: 30, nucleotides 2109-2408 of SEQ ID NO: 31, nucleotides 2109-2426 of SEQ ID NO: 32, or nucleotides 2109-2408 of SEQ ID NO: 33. In some embodiments, the siRNA includes a sense strand and an antisense strand selected from the following pairs: SEQ ID NO: 35 and SEQ ID NO: 36; SEQ ID NO: 37 and SEQ ID NO: 38; SEQ ID NO: 39 and SEQ ID NO: 40; and SEQ ID NO: 41 and SEQ ID NO: 42. In some embodiments, the inhibitory RNA is an miRNA. In some embodiments, the miRNA is human miR-145, miR-126, miR-200c, miR-429, miR-200b, miR-140, miR-9, miR-21, miR-590, miR-182, or miR-638, or murine miR-134, miR-200c, miR-429, miR-200b, miR-34a, or miR-9.

In some embodiments of any of the foregoing aspects, the Sox2 inhibitor is an inhibitory RNA molecule targeting a Sox2 promoter. In some embodiments, the inhibitory RNA molecule is an miRNA.

In some embodiments of any of the foregoing aspects, the Sox2 inhibitor is a component of a gene editing system targeting Sox2. In some embodiments, the gene editing system is a zinc finger nuclease (ZFN) system, a transcription activator-like effector-based nuclease (TALEN) system, or a clustered regulatory interspaced short palindromic repeat (CRISPR) system.

In some embodiments of any of the foregoing aspects, the Sox2 inhibitor is a dominant negative Sox2 protein or a polynucleotide encoding a dominant negative Sox2 protein. In some embodiments, the polynucleotide encoding the dominant negative Sox2 protein has the sequence of SEQ ID NO: 24. In some embodiments, the polynucleotide encoding the dominant negative Sox2 protein has the sequence of SEQ ID NO: 34. In some embodiments, the dominant negative Sox2 protein is a Sox2 protein that lacks most or all of the high mobility group domain (HMGD), a Sox2 protein in which the nuclear localization signals in the HMGD are mutated, a Sox2 protein in which the HMGD is fused to an engrailed repressor domain, or a c-terminally truncated Sox2 protein comprising only the DNA binding domain.

In some embodiments of any of the foregoing aspects, the method further includes administering a regeneration agent. In some embodiments, the regeneration agent is administered before the Sox2 inhibitor. In some embodiments, the regeneration agent is administered after the Sox2 inhibitor. In some embodiments, the regeneration agent is administered concurrently with the Sox2 inhibitor.

In some embodiments of any of the foregoing aspects, the nucleic acid vector further includes a regeneration agent.

In some embodiments of any of the foregoing aspects, the regeneration agent is an agent that increases Atoh1 expression and/or a Notch inhibitor. In some embodiments, the regeneration agent is an agent that increases Atoh1 expression. In some embodiments, the agent that increases Atoh1 expression is a polynucleotide encoding Atoh1 (e.g., a polynucleotide encoding SEQ ID NO: 43, such as a polynucleotide having the sequence of SEQ ID NO: 44). In some embodiments, the agent that increases Atoh1 expression is a small molecule. In some embodiments, the regeneration agent is a Notch inhibitor. In some embodiments, the Notch inhibitor is an inhibitory RNA targeting Notch, a small molecule Notch inhibitor (e.g., a gamma-secretase inhibitor), an anti-Notch antibody, or a polynucleotide encoding an anti-Notch antibody. In some embodiments, the Notch inhibitor is an inhibitory RNA targeting Notch. In some embodiments, the inhibitory RNA targeting Notch is an siRNA. In some embodiments, the inhibitory RNA targeting Notch is an shRNA. In some embodiments, the inhibitory RNA targeting Notch is an miRNA. In some embodiments, the Notch inhibitor is a small molecule Notch inhibitor. In some embodiments, the Notch inhibitor is an anti-Notch antibody. In some embodiments, the Notch inhibitor is a polynucleotide encoding an anti-Notch antibody.

In some embodiments of any of the foregoing aspects, the regeneration agent is administered using a nucleic acid vector.

In some embodiments of any of the foregoing aspects, the nucleic acid vector includes a promoter operably linked to the regeneration agent. In some embodiments, the regeneration agent is a polynucleotide encoding Atoh1, an siRNA targeting Notch, an shRNA targeting Notch, an miRNA targeting Notch, or a polynucleotide encoding an anti-Notch antibody and the promoter is a pol II promoter. In some embodiments, the pol II promoter is a supporting cell promoter. In some embodiments, the supporting cell promoter is a Glial Acidic Fibrillary Protein (GFAP) promoter, a Solute Carrier Family 1 Member 3 (GLAST) promoter, a Hes Family BHLH Transcription Factor 1 (HES1) promoter, a Jagged 1 (JAG1) promoter, a Notch 1 (NOTCH1) promoter, a Leucine Rich Repeat Containing G Protein-Coupled Receptor 5 (LGR5) promoter, a SOX2 promoter, a Hes Family BHLH Transcription Factor 5 (HESS) promoter, a LFNG 0-Fucosylpeptide 3-Beta-N-Acetylglucosaminyltransferase (LFNG) promoter, a Kringle Containing Transmembrane Protein 1 (KREMEN1) promoter, an Anterior Gradient 3, Protein Disulphide Isomerase Family Member (AGR3) promoter, a SRY-Box 9 (SOX9), or a Solute Carrier Family 6 Member 14 (SLC6A14) promoter. In some embodiments, regeneration agent is an siRNA targeting Notch, an shRNA targeting Notch, or an miRNA targeting Notch and the promoter is a pol III promoter.

In some embodiments of any of the foregoing aspects, the Sox2 inhibitor is administered using a nucleic acid vector.

In some embodiments of any of the foregoing aspects, the nucleic acid vector includes a promoter operably linked to the Sox2 inhibitor. In some embodiments of any of the foregoing aspects, the Sox2 inhibitor is an siRNA targeting Sox2 or a Sox2 promoter, an shRNA targeting Sox2 or a Sox2 promoter, an shRNA targeting Sox2 or a Sox2 promoter embedded in an miRNA (an shRNA-mir), an miRNA targeting Sox2, or an miRNA targeting a Sox2 promoter and the promoter is a pol III promoter. In some embodiments of any of the foregoing aspects, the Sox2 inhibitor is an siRNA targeting Sox2 or a Sox2 promoter, an shRNA targeting Sox2 or a Sox2 promoter, an shRNA targeting Sox2 or a Sox2 promoter embedded in an miRNA, an miRNA targeting Sox2, an miRNA targeting a Sox2 promoter, a polynucleotide encoding component of a gene editing system targeting Sox2, or a polynucleotide encoding the dominant negative Sox2 protein and the promoter is a pol II promoter. In some embodiments, the pol II promoter is a hair cell promoter. In some embodiments, the hair cell promoter is a Myosin 15A (MYO15) promoter, a Growth Factor Independent 1 Transcriptional Repressor (GFI1) promoter, a POU Class 4 Homeobox 3 (POU4F3) promoter, or Myosin 7a (MYO7A) promoter. In some embodiments, the pol II promoter is a Type II vestibular hair cell promoter. In some embodiments, the Type II vestibular hair cell promoter is a Calbindin 2 (CALB2) promoter, a Microtubule associated protein tau (MAPT) promoter, an Annexin A4 (ANXA4) promoter, or an Otoferlin (OTOF) promoter.

In some embodiments of any of the foregoing aspects, the pol III promoter is a ubiquitous pol III promoter. In some embodiments, the ubiquitous pol III promoter is a U6 promoter, an H1 promoter, or a 7SK promoter.

In some embodiments of any of the foregoing aspects, the pol II promoter is a ubiquitous promoter. In some embodiments, the ubiquitous pol II promoter is a CMV promoter, a CAG promoter, or a smCBA promoter.

In some embodiments of any of the foregoing aspects, the Sox2 inhibitor and the regeneration agent are administered using separate nucleic acid vectors.

In some embodiments of any of the foregoing aspects, the Sox2 inhibitor and the regeneration agent are administered using a single nucleic acid vector that expresses both the Sox2 inhibitor and the regeneration agent.

In some embodiments of any of the foregoing aspects, the Sox2 inhibitor and the regeneration agent are expressed using two different promoters. In some embodiments of any of the foregoing aspects, the Sox2 inhibitor and the regeneration agent are expressed using the same promoter.

In some embodiments of any of the foregoing aspects, the regeneration agent is a polynucleotide encoding Atoh1.

In some embodiments of any of the foregoing aspects, the nucleic acid vector is a plasmid, cosmid, artificial chromosome, or viral vector.

In some embodiments of any of the foregoing aspects, the nucleic acid vector is a viral vector. In some embodiments, the viral vector is selected from the group consisting of an adeno-associated virus (AAV), an adenovirus, and a lentivirus. In some embodiments, the viral vector is an AAV vector. In some embodiments, the AAV viral vector has an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ, DJ/8, DJ/9, 7m8, PHP.B, PHP.B2, PBP.B3, PHP.A, PHP.eb, or PHP.S capsid. In some embodiments, the AAV viral vector has an AAV1 capsid. In some embodiments, the AAV viral vector has an AAV2 capsid. In some embodiments, the AAV viral vector has an AAV8 capsid. In some embodiments, the AAV viral vector has an AAV9 capsid. In some embodiments, the AAV viral vector has an Anc80 capsid. In some embodiments, the AAV viral vector has a DJ capsid. In some embodiments, the AAV viral vector has a 7m8 capsid. In some embodiments, the AAV viral vector has a PHP.B capsid. In some embodiments, the AAV viral vector has a PHP.B2 capsid. In some embodiments, the AAV viral vector has a PHP.B3 capsid. In some embodiments, the AAV viral vector has a PHP.A capsid. In some embodiments, the AAV viral vector has a PHP.eb capsid. In some embodiments, the AAV viral vector has a PHP.S capsid.

It will be understood by those of skill in the art, that a diagnosis of vestibular dysfunction can only be made in a subject with a mature vestibular system (e.g., a vestibular system that has completed the development process that occurs during a term pregnancy, which is defined as the onset of labor at 37 weeks or later (e.g., 37 weeks, 38 weeks, 39 weeks, 40 weeks, 41 weeks, 42 weeks, or later) in humans). Accordingly, the term “human subject” as used herein, refers to human adults, adolescents, children, infants, and term newborns. In some embodiments of any of the foregoing aspects, the subject is an adult. In some embodiments of any of the foregoing aspects, the subject is an adolescent. In some embodiments of any of the foregoing aspects, the subject is a child. In some embodiments of any of the foregoing aspects, the subject is an infant. In some embodiments of any of the foregoing aspects, the subject is a term newborn.

In some embodiments of any of the foregoing aspects, the vestibular dysfunction comprises vertigo, dizziness, loss of balance (imbalance), bilateral vestibulopathy (bilateral vestibular hypofunction), oscillopsia, or a balance disorder. In some embodiments, the vestibular dysfunction comprises loss of balance. In some embodiments, the vestibular dysfunction comprises vertigo. In some embodiments, the vestibular dysfunction comprises dizziness. In some embodiments, the vestibular dysfunction comprises bilateral vestibulopathy. In some embodiments, the vestibular dysfunction comprises oscillopsia. In some embodiments, the vestibular dysfunction comprises a balance disorder.

In some embodiments of any of the foregoing aspects, the vestibular dysfunction is age-related vestibular dysfunction, head trauma-related vestibular dysfunction, disease or infection-related vestibular dysfunction, or ototoxic drug-induced vestibular dysfunction. In some embodiments, the ototoxic drug is an aminoglycoside, an antineoplastic drug, ethacrynic acid, furosemide, a salicylate, or quinine. In some embodiments, the vestibular dysfunction is due to aminoglycoside ototoxicity. In some embodiments, the vestibular dysfunction is bilateral vestibulopathy due to aminoglycoside ototoxicity. In some embodiments, the vestibular dysfunction is oscillopsia due to aminoglycoside ototoxicity.

In some embodiments of any of the foregoing aspects, the vestibular dysfunction is associated with a genetic mutation.

In some embodiments of any of the foregoing aspects, the vestibular dysfunction is idiopathic vestibular dysfunction.

In some embodiments of any of the foregoing aspects, the method further comprises evaluating the vestibular function of the subject prior to administering the nucleic acid vector or composition.

In some embodiments of any of the foregoing aspects, the method further comprises evaluating the vestibular function of the subject after administering the nucleic acid vector or composition.

In some embodiments of any of the foregoing aspects, the Sox2 inhibitor and/or regeneration agent is locally administered. In some embodiments, the Sox2 inhibitor is administered to the middle or inner ear (e.g., into the perilymph or endolymph, such as through the oval window, round window, or semicircular canal (e.g., the horizontal canal)). In some embodiments, the Sox2 inhibitor and/or regeneration agent is administered to a semicircular canal (e.g., intra-labyrinth delivery). In some embodiments, the Sox2 inhibitor and/or regeneration agent is administered to or through the oval window.

In some embodiments, the Sox2 inhibitor and/or regeneration agent is administered to or through the round window. In some embodiments, the Sox2 inhibitor and/or regeneration agent is administered by transtympanic or intratympanic injection.

In some embodiments of any of the foregoing aspects, the Sox2 inhibitor decreases the expression or activity of Sox2.

In some embodiments of any of the foregoing aspects, the Sox2 inhibitor and/or regeneration agent is administered in an amount sufficient to prevent or reduce vestibular dysfunction, delay the development of vestibular dysfunction, slow the progression of vestibular dysfunction, improve vestibular function, improve balance, reduce dizziness, reduce vertigo, increase Type I vestibular hair cell numbers, increase the generation of Type I vestibular hair cells, or promote or increase vestibular hair cell regeneration.

In some embodiments of any of the foregoing aspects, the Sox2 inhibitor and/or regeneration agent increases the generation of Type I vestibular hair cells, increases the number of Type I vestibular hair cells, and/or induces or increases hair cell regeneration in the striolar region, in the extrastriolar region, or in both the striolar and extrastriolar regions of one or more vestibular organs (e.g., the utricle and/or the crista). In some embodiments, the Sox2 inhibitor and/or regeneration agent increases the generation of Type I vestibular hair cells, increases the number of Type I vestibular hair cells, and/or induces or increases hair cell regeneration in the striolar region of one or more vestibular organs. In some embodiments, the Sox2 inhibitor and/or regeneration agent increases the generation of Type I vestibular hair cells, increases the number of Type I vestibular hair cells, and/or induces or increases hair cell regeneration in the extrastriolar region of one or more vestibular organs. In some embodiments, the Sox2 inhibitor and/or regeneration agent increases the generation of Type I vestibular hair cells, increases the number of Type I vestibular hair cells, and/or induces or increases hair cell regeneration in both the striolar and extrastriolar regions of one or more vestibular organs.

In some embodiments of any of the foregoing aspects, the Sox2 inhibitor and/or regeneration agent increases the generation of Type I vestibular hair cells, increases the number of Type I vestibular hair cells, and/or induces or increases hair cell regeneration in the crista. In some embodiments of any of the foregoing aspects, the Sox2 inhibitor and/or regeneration agent increases the generation of Type I vestibular hair cells, increases the number of Type I vestibular hair cells, and/or induces or increases hair cell regeneration in the utricle. In some embodiments of any of the foregoing aspects, the Sox2 inhibitor and/or regeneration agent increases the generation of Type I vestibular hair cells, increases the number of Type I vestibular hair cells, and/or induces or increases hair cell regeneration in both the crista and the utricle.

In some embodiments of any of the foregoing aspects, the subject is a human.

### BRIEF DESCRIPTION OF THE DRAWINGS

FIG. 1 shows micrograph data of Sox2 expression levels in normal and Sox2 knockout mouse vestibular hair cells. Expression of the Cre transgene and Sox2 protein in vestibular hair cells of C57Bl6 and Sox2^{fl/fl} mice transformed with an AAV8-Cre vector were visualized using immunohistochemistry specific for each protein.

FIG. 2 shows two images of an inner ear vestibular organ section from a mouse transduced with an AAV8 vector carrying a Woodchuck Hepatitis Virus Posttranscriptional Regulatory Element (WPRE) and labeled with a DNAScope probe specific for the WPRE. The left image is stained with hematoxylin in addition to the DNAScope probe. The right image is visualized only with the DNAScope probe.

FIGS. 3A-3B are a series of images (FIG. 3A) and corresponding quantification (FIG. 3B) showing Cre and Tuj expression in the vestibular cells of both C57Bl6 and naïve Sox2^{fl/fl} mice, each transduced with a Cre-expressing AAV8 vector (inducing Sox2 knockout). FIG. 3A shows micrographs of sections of vestibular hair cells visualized using immunohistochemistry specific for each protein. Asterisks depict native vestibular type I hair cells characterized by the lack of Cre and presence of a Tuj+ calyx. FIG. 3B is a bar graph showing quantification of Cre+/Tuj+ hair cells from each mouse type. The Sox2^{fl/fl} vestibular tissue treated with AAV8-Cre showed a statistically significant increase in the percentage of Cre+ nuclei with calyxes compared to C57Bl6 control tissue (n=3, Student's t-test, p<0.005).

FIGS. 4A-4B are a series of violin plots, generated from scRNAseq data. FIG. 4A demonstrates changes to different gene expression levels in type I, converting type II, and native type II vestibular hair cells after Sox2 knockout via AAV8-Cre transduction in naïve Sox2^{fl/fl} mice. Type I hair cell genes (Kcna10, Rarb, and Lpgat1) are shown in the top row. Type II hair cell genes (Mgst3, DIk2 and Kcnv1) are shown in the bottom row. FIG. 4B shows Sox2 expression in type I, converting type II, and native type II vestibular hair cells in Sox2^{fl/fl} mice transformed with an AAV8 vector encoding Cre, which, upon expression, will delete the Sox2 gene in the Sox2^{fl/fl} mice.

FIGS. 5A-5C are a series of images (FIGS. 5A and 5C) and corresponding quantification (FIG. 5B) showing Cre and Tuj expression in the vestibular cells of IDPN-treated Sox2^{fl/fl} mice transduced with either an AAV8-Cre (inducing Sox2 knockout) or an AAV-GFP vector (as a negative control). FIG. 5A shows micrographs of sections of vestibular hair cells visualized using immunohistochemistry specific for each protein. FIG. 3B is a bar graph showing quantification of Cre+/Tuj+ hair cells from each mouse type. The Sox2^{fl/fl} vestibular tissue treated with AAV8-Cre showed a statistically significant increase in the percentage of Cre+ nuclei with calyxes compared to tissue treated with AAV-GFP (n=4, Student's t-test, p<0.0001). FIG. 5C shows micrographs of sections of vestibular hair cells in the striolar and extrastriolar regions of the utricle sensory epithelium using immunohistochemistry specific for Cre or Tuj. Arrows point to Cre+ nuclei of converting hair cells surrounded by a Tuj calyx (FIGS. 5A and 5C).

FIG. 6 is a series of violin plots, generated from scRNAseq data, demonstrating changes to different gene expression levels in the cristae of Sox2 fl/fl mice treated with the hair cell damaging agent 3,3′-iminodipropionitrile (IDPN) and then transduced with either AAV8-Cre or AAV8-GFP. Type I hair cell genes (Kcna10, Rarb, and Lpgat1) are shown in the top row. Type II hair cell genes (Mgst3, DIk2 and Kcnv1) are shown in the bottom row.

FIG. 7 is a series of violin plots, generated from scRNAseq data, demonstrating changes to gene expression levels in pre-existing and regenerated type II vestibular cells in Sox2 fl/fl mice treated with IDPN and then transduced with an AAV8 vector co-expressing ATOH1 and GFP alone or together with an AAV8-Cre vector (“Sox2 KO”). Type I hair cell genes (Kcna10, Rarb, and Lpgat1) are shown in the top row. Type II hair cell genes (Mgst3, DIk2 and Kcnv1) are shown in the bottom row.

FIGS. 8A-8B are a series of graphs showing the percentage knockdown (KD) of Sox2 mRNA levels in P19 cells following transfection with plasmids encoding GFP and different Sox2-specific shRNAs or a control shRNA scaffolded with either mir30 or mirE. FIG. 8A depicts Sox2 mRNA knockdown in P19 cells transformed with a plasmid encoding having either a high (“hi”) or low (“10”) level of GFP expression after transfection with a plasmid encoding Sox2-specific shRNA4 scaffolded with mir30 (P799), Sox2-specific shRNA2 scaffolded with mirE (P900) or Sox2-specific shRNA4 scaffolded with mirE (P901). FIG. 8B depicts Sox2 mRNA knockdown in P19 cells having high GFP expression following transfection with a plasmid containing Sox2-specific shRNA2 scaffolded with either mir30 (P797) or mirE (P900), as well as plasmids containing a scrambled negative control shRNA scaffolded with either mir30 (P857) or mirE (P858).

FIG. 9 is a graph showing the percentage knockdown (KD) of Sox2 mRNA levels in adult mouse C57BL/6J utricle sensory epithelia after culturing in the presence of a group of Sox2-targeting siRNAs (Sox2 siRNA A-058489-13, Sox2 siRNA A-058489-14, Sox2 siRNA A-058489-15, and Sox2 siRNA A-058489-16) or non-targeting siRNAs at 1 μM for 72 hours. Sox2 expression across all groups (n=5 utricles per condition) was normalized to an endogenous control gene, Actb, and percentage of Sox2 knockdown compared to a non-targeting control siRNA group.

FIG. 10 is a series of micrographs of P19 cells transduced with a viral vector encoding dnSOX2-FLAG. FIG. 10 shows the same field of cells in the left and right panel. The left panel shows cells that are SOX2+ by immunostaining with an antibody that recognizes native SOX2, but not dnSOX2. The right panel shows cells that are dnSOX2-FLAG+ by immunostaining with an antibody that recognizes FLAG. Arrowheads are cell nuclei positive for SOX2+, but that do not contain FLAG. Arrows indicate cells that were both SOX2+ and dnSOX2-FLAG+.

FIG. 11 is a series of violin plots, generated from scRNAseq data, showing the expression levels of six type II HC marker genes at different embryonic (E) and post-natal (P) ages in naïve mice. Adult mice are >P50.

FIGS. 12A-12B are a series of micrographs showing Pou4f3 and Spp1 protein expression in utricle explants treated with vitamin A (top rows of FIGS. 12A and 12B) or a control (bottom rows of FIGS. 12A and 12B). FIG. 12A shows a series of micrographs from the same field of cells from an E15.5 embryonic utricle explant. FIG. 12B shows a series of micrographs from the same field of cells from adult utricle explants shown at two different magnifications (first two top and bottom panels vs last two top and bottom panels) cultured for 14 days in 0.1 μM vitamin A or a negative control. Circles in each of FIGS. 12A and 12B indicate Pou4f3-positive hair cells expressing SPP1.

FIGS. 13A-13B are a series of violin plots, generated from scRNAseq data, showing the expression levels of type I (Kcna10 and Lpgat1) and type II (Mgst3 and DIk2) hair cell marker genes caused by retinoic acid pathway modulation in hair cells of embryonic (FIG. 13A) and adult (FIG. 13B) utricle cultures. FIG. 13A shows the expression of these genes with (4 DIV-vitA) and without (4 DIV-Ctrl) the addition of 0.1 μM vitamin A to cultured embryonic utricles from E15.5 CD1 mice. FIG. 13B shows the expression of these genes in utricle explants from adult mice cultured in the presence of 1 μM retinoic acid after transduction with either a combination of AAV8 vectors driving expression of the retinoic acid receptors Rarb and Rxra (“AAV-Rxra-Rarb”), or with a control AAV8 vector expressing GFP (“AAV-GFP”).

### DEFINITIONS

As used herein, “administration” refers to providing or giving a subject a therapeutic agent (e.g., an agent that reduces Sox2 activity or expression), by any effective route. Exemplary routes of administration are described herein below.

As used herein, the term “cell type” refers to a group of cells sharing a phenotype that is statistically separable based on gene expression data. For instance, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, the terms “complementarity” or “complementary” of nucleic acids means that a nucleotide sequence in one strand of nucleic acid, due to orientation of its nucleobase groups, forms hydrogen bonds with another sequence on an opposing nucleic acid strand. The complementary bases in DNA are typically A with T and C with G. In RNA, they are typically C with G and U with A. Complementarity can be perfect or substantial/sufficient. Perfect complementarity between two nucleic acids means that the two nucleic acids can form a duplex in which every base in the duplex is bonded to a complementary base by Watson-Crick pairing. “Substantial” or “sufficient” complementary means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations to predict the Tm (melting temperature) of hybridized strands, or by empirical determination of Tm by using routine methods. Tm includes the temperature at which a population of hybridization complexes formed between two nucleic acid strands are 50% denatured (i.e., a population of double-stranded nucleic acid molecules becomes half dissociated into single strands). At a temperature below the Tm, formation of a hybridization complex is favored, whereas at a temperature above the Tm, melting or separation of the strands in the hybridization complex is favored. Tm may be estimated for a nucleic acid having a known G+C content in an aqueous 1 M NaCl solution by using, e.g., Tm=81.5+0.41(% G+C), although other known Tm computations take into account nucleic acid structural characteristics.

As used herein, the term “conservative amino acid substitution” refers to a substitution of one or more amino acids for one or more different amino acids that exhibit similar physicochemical properties, such as polarity, electrostatic charge, and steric volume. These properties are summarized for each of the twenty naturally-occurring amino acids in table 1 below.

TABLE 1
Representative physicochemical properties
of naturally-occurring amino acids

| | | | | Electrostatic | |
|---|---|---|---|---|---|
| | 3 | 1 | Side- | character at | |
| | Letter | Letter | chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral | large |
| | | | | and cationic | |
| | | | | forms in | |
| | | | | equilibrium | |
| | | | | at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | nonpolar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include (i) G, A, V, L and I; (ii) D and E; (iii) C, S and T; (iv) H, K and R; (v) N and Q; and (vi) F, Y and W. A conservative amino acid substitution is therefore one that substitutes one amino acid for a member of the same amino acid family (e.g., a substitution of Ser for Thr or Lys for Arg).

As used herein, the terms “effective amount,” “therapeutically effective amount,” and a “sufficient amount” of a composition, vector construct, or viral vector described herein refer to a quantity sufficient to, when administered to the subject, including a mammal, for example a human, effect beneficial or desired results, including clinical results, and, as such, an “effective amount” or synonym thereto depends upon the context in which it is being applied. For example, in the context of treating vestibular dysfunction, it is an amount of the composition, vector construct, or viral vector sufficient to achieve a treatment response as compared to the response obtained without administration of the composition, vector construct, or viral vector. The amount of a given composition described herein that will correspond to such an amount will vary depending upon various factors, such as the given agent, the pharmaceutical formulation, the route of administration, the type of disease or disorder, the identity of the subject (e.g. age, sex, weight) or host being treated, and the like, but can nevertheless be routinely determined by one skilled in the art. Also, as used herein, a “therapeutically effective amount” of a composition, vector construct, or viral vector of the present disclosure is an amount which results in a beneficial or desired result in a subject as compared to a control. As defined herein, a therapeutically effective amount of a composition, vector construct, or viral vector of the present disclosure may be readily determined by one of ordinary skill by routine methods known in the art. Dosage regimen may be adjusted to provide the optimum therapeutic response.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell, e.g., a human vestibular supporting cell).

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell, e.g., a human vestibular supporting cell). Exogenous materials include those that are provided from an external source to an organism or to cultured matter extracted there from.

As used herein, the term “exon” refers to a region within the coding region of a gene, the nucleotide sequence of which determines the amino acid sequence of the corresponding protein. The term exon also refers to the corresponding region of the RNA transcribed from a gene. Exons are transcribed into pre-mRNA, and may be included in the mature mRNA depending on the alternative splicing of the gene. Exons that are included in the mature mRNA following processing are translated into protein, wherein the sequence of the exon determines the amino acid composition of the protein.

As used herein, the term “heterologous” refers to a combination of elements that is not naturally occurring. For example, a heterologous transgene refers to a transgene that is not naturally expressed by the promoter to which it is operably linked.

As used herein, the terms “increasing” and “decreasing” refer to modulating resulting in, respectively, greater or lesser amounts, of function, expression, or activity of a metric relative to a reference. For example, subsequent to administration of a composition in a method described herein, the amount of a marker of a metric (e.g., Sox2 expression) as described herein may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the marker prior to administration. Generally, the metric is measured subsequent to administration at a time that the administration has had the recited effect, e.g., at least one week, one month, 3 months, or 6 months, after a treatment regimen has begun.

As used herein, the term “linker” refers to a series of nucleotides that connects two different regions of a polynucleotide. A linker is functionally inert and does not disrupt the function of the two regions of the polynucleotide that it connects.

As used herein, “locally” or “local administration” means administration at a particular site of the body intended for a local effect and not a systemic effect. Examples of local administration are epicutaneous, inhalational, intra-articular, intrathecal, intravaginal, intravitreal, intrauterine, intra-lesional administration, lymph node administration, intratumoral administration, administration to the middle or inner ear, and administration to a mucous membrane of the subject, wherein the administration is intended to have a local and not a systemic effect.

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. Additionally, two portions of a transcription regulatory element are operably linked to one another if they are joined such that the transcription-activating functionality of one portion is not adversely affected by the presence of the other portion. Two transcription regulatory elements may be operably linked to one another by way of a linker nucleic acid (e.g., an intervening non-coding nucleic acid) or may be operably linked to one another with no intervening nucleotides present.

As used herein, the term “plasmid” refers to a to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, the term “polynucleotide” refers to a polymer of nucleosides. Typically a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. The term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e., the succession of letters used as abbreviations for bases) that biochemically characterizes a specific nucleic acid. A polynucleotide sequence presented herein is presented in a 5′ to 3′ direction unless otherwise indicated.

As used herein, the term “promoter” refers to a recognition site on DNA that is bound by an RNA polymerase. The polymerase drives transcription of the transgene.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “pharmaceutical composition” refers to a mixture containing a therapeutic agent, optionally in combination with one or more pharmaceutically acceptable excipients, diluents, and/or carriers, to be administered to a subject, such as a mammal, e.g., a human, in order to prevent, treat or control a particular disease or condition affecting or that may affect the subject.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the term “regeneration agent” refers to an agent that promotes the regeneration of hair cells from supporting cells. Regeneration agents include agents that increase the expression (e.g., lead to overexpression) of Atonal BHLH transcription factor 1 (Atoh1) in vestibular supporting cells. Atoh1 can be overexpressed using a vector, e.g., a viral vector, such as an AAV vector, adenoviral vector, or lentiviral vector, containing a polynucleotide encoding Atoh1. Atoh1 expression can also be increased using one or more small molecules found to promote regeneration (e.g., using a method described in U.S. Pat. No. 8,188,131 or 10,143,711 or in U.S. Patent Application Nos. US20170042842 and US20190203210). Regeneration agents also include agents that inhibit Notch in vestibular supporting cells, such as small molecule inhibitors (e.g., gamma-secretase inhibitors), inhibitory RNA molecules (e.g., siRNA, shRNA, or miRNA) directed to Notch or to the Notch promoter, or anti-Notch antibodies. Agents that increase Atoh1 expression and agents that inhibit Notch can be used separately or in combination.

As used herein, the term “Sox2 inhibitor” refers to an agent that reduces Sox2 activity or expression. Sox2 inhibitors include inhibitory RNA molecules (e.g., siRNA, shRNA, or miRNA) directed to Sox2 mRNA or to a Sox2 promoter, components of gene editing systems that target Sox2 (e.g., gene editing systems such as CRISPR, ZFN, and TALEN-based systems), and dominant negative Sox2 proteins.

As used herein, the term “transcription regulatory element” refers to a nucleic acid that controls, at least in part, the transcription of a gene of interest. Transcription regulatory elements may include promoters, enhancers, and other nucleic acids (e.g., polyadenylation signals) that control or help to control gene transcription. Examples of transcription regulatory elements are described, for example, in Lorence, Recombinant Gene Expression: Reviews and Protocols (Humana Press, New York, N.Y., 2012).

As used herein, the term “transfection” refers to any of a wide variety of techniques commonly used for the introduction of exogenous DNA into a prokaryotic or eukaryotic host cell, e.g., electroporation, lipofection, calcium phosphate precipitation, DEAE-dextran transfection, Nucleofection, squeeze-poration, sonoporation, optical transfection, magnetofection, impalefection and the like.

As used herein, the terms “subject” and “patient” refer to an animal (e.g., a mammal, such as a human). A subject to be treated according to the methods described herein may be one who has been diagnosed with vestibular dysfunction (e.g., dizziness, vertigo, imbalance or loss of balance, bilateral vestibulopathy (bilateral vestibular hypofunction), oscillopsia, or a balance disorder) or one at risk of developing these conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the terms “transduction” and “transduce” refer to a method of introducing a vector construct or a part thereof into a cell. Wherein the vector construct is contained in a viral vector such as for example an AAV vector, transduction refers to viral infection of the cell and subsequent transfer and integration of the vector construct or part thereof into the cell genome.

As used herein, “treatment” and “treating” in reference to a disease or condition, refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “vector” includes a nucleic acid vector, e.g., a DNA vector, such as a plasmid, cosmid, or artificial chromosome, an RNA vector, a virus, or any other suitable replicon (e.g., viral vector). A variety of vectors have been developed for the delivery of polynucleotides encoding exogenous proteins into a prokaryotic or eukaryotic cell. Examples of such expression vectors are described in, e.g., Gellissen, Production of Recombinant Proteins: Novel Microbial and Eukaryotic Expression Systems (John Wiley & Sons, Marblehead, M A, 2006). Expression vectors suitable for use with the compositions and methods described herein contain a polynucleotide sequence as well as, e.g., additional sequence elements used for the expression of proteins and/or the integration of these polynucleotide sequences into the genome of a mammalian cell. Certain vectors that can be used for the expression of transgene as described herein include vectors that contain regulatory sequences, such as promoter and enhancer regions, which direct gene transcription. Other useful vectors for expression of a transgene contain polynucleotide sequences that enhance the rate of translation of the transgene or improve the stability or nuclear export of the mRNA that results from gene transcription. These sequence elements include, e.g., 5′ and 3′ untranslated regions and a polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the term “vestibular hair cell” refers to group of specialized cells in the inner ear that are involved in sensing movement and contribute to the sense of balance and spatial orientation. There are two types of vestibular hair cells: Type I and Type II hair cells. Vestibular hair cells are located in the semicircular canal end organs and otolith organs of the inner ear. Damage to vestibular hair cells and genetic mutations that disrupt vestibular hair cell function are implicated in vestibular dysfunction such as vertigo and imbalance disorders.

As used herein, the term “vestibular supporting cell” refers to a collection of specialized epithelial cells in the vestibular system of the inner ear that are involved in vestibular hair cell development, survival, function, death, and phagocytosis. Vestibular supporting cells provide structural support to vestibular hair cells by anchoring them in the sensory epithelium and releasing neurotrophic factors important for hair cell innervation.

As used herein, the term “wild-type” refers to a genotype with the highest frequency for a particular gene in a given organism.

### DETAILED DESCRIPTION

Described herein are compositions and methods for reducing Sox2 activity or expression in vestibular hair cells of the inner ear. The invention features Sox2 inhibitors (e.g., agents that reduce Sox2 activity or expression, such as inhibitory RNA directed to Sox2, nuclease systems directed to Sox2, and dominant negative Sox2 protein). The compositions and methods described herein can be used to convert Type II vestibular hair cells or regenerated vestibular hair cells into Type I vestibular hair cells. Accordingly, the compositions described herein can be administered to a subject (such as a mammalian subject, for example, a human) to treat disorders caused by dysfunction of vestibular hair cells, such as balance loss.

### Vestibular Hair Cells

Hair cells are sensory cells of the auditory and vestibular systems that reside in the inner ear. Cochlear hair cells are the sensory cells of the auditory system and are made up of two main cell types: inner hair cells, which are responsible for sensing sound, and outer hair cells, which are thought to amplify low-level sound. Vestibular hair cells are located in the semicircular canal end organs and otolith organs of the inner ear and are involved in the sensation of movement that contributes to the sense of balance and spatial orientation. Hair cells are named for the stereocilia that protrude from the apical surface of the cell, forming a hair cell bundle. Deflection of the stereocilia (e.g., by sound waves in cochlear hair cells, or by rotation or linear acceleration in vestibular hair cells) leads to the opening of mechanically gated ion channels, which allows hair cells to release neurotransmitters to activate nerves, thereby converting mechanical sound or motion signals into electrical signals that can be transmitted to the brain. Cochlear hair cells are essential for normal hearing, and damage to cochlear hair cells and genetic mutations that disrupt cochlear hair cell function are implicated in hearing loss and deafness. Damage to vestibular hair cells and genetic mutations that disrupt vestibular hair cell function are implicated in vestibular dysfunction, such as loss of balance, vertigo, dizziness, bilateral vestibulopathy (bilateral vestibular hypofunction), and oscillopsia.

The vestibular system contains two types of hair cells: Type I hair cells, which have a flask morphology, long stereocilia, and calyceal nerve terminals, and Type II hair cells, which have a cylindrical morphology, short stereocilia, and small bouton terminals. Type I and Type II hair cells are found in both central (referred to as the striola in the utricle and saccule) and extrastriolar/peripheral zones of all five vestibular organs, usually in roughly equal ratios, of all mammals that have been examined, including humans. Accumulating evidence indicates that Type I hair cells, particularly those located centrally within the sensory epithelium, may be better suited for the detection of high frequency head movements compared to Type II hair cells (Burns and Stone, supra).

Type I hair cells are classically defined by the presence of cup-shaped, calyceal afferent innervation, whereas Type II hair cells synapse upon discrete bouton afferent terminals. Distinct morphological differences such as cell shape and stereocilia width and length have also been found to differentiate each subtype. At the molecular level, differences in Sox2 transcription factor expression (expressed in Type II hair cells but not Type I hair cells) and calcium binding protein expression can be reliable indicators of hair cell subtypes (Oesterle et al., 2008. J. Assoc. Res. Otolaryngol. 9(1):65-89). High throughput profiling methods like single-cell RNA-sequencing have allowed for characterization of differences at the whole transcriptome level, and there is a growing list of molecular markers that separate Type I and Type II hair cells, as well as hair cells in striolar/central and extrastriolar/peripheral zones. At the electrophysiological level, Type I hair cells possess a unique, outwardly rectifying low-voltage-activated potassium conductance called gKL. This feature makes Type I hair cells particularly well-suited to detect higher frequency stimuli with little phase delay, which is likely essential for driving fast reflexes (Burns and Stone, supra).

Type II hair cells appear to be less differentiated than Type I hair cells based on electrophysiology, innervation, morphology, and gene expression. Furthermore, single-cell RNA-Seq shows that expression of many supporting cell-specific genes persists in Type II hair cells compared to other, more differentiated hair cells. Although the functional role of Type I and II hair cells is still being elucidated, some evidence suggests that Type I hair cells might encode most motions relevant to balance function in humans. Regeneration of Type I hair cells could therefore have a profound impact on recovery of lost vestibular function. The process of generating new Type I hair cells can also result in the recruitment of new calyceal nerve endings to those cells. Calyces are specialized afferent terminals that transmit signals from Type I hair cells to the brain.

Based on several studies, the development of the vestibular organs is known to begin as patches of sensory epithelia located in the mid-ventral region of the embryonic inner ear. Some cells within these sensory patches become post-mitotic as early as E1l in the mouse and the first differentiating hair cells can be seen by E12. Terminal mitoses begin in the striolar/central portions of the sensory epithelia, and then by 3-4 days later most cell cycle exit occurs at the periphery. Mitotic addition of new hair cells at the periphery of the sensory region continues for 2-4 days after birth, at least in the utricle. In parallel, the total number of hair cells in the sensory epithelium continues to increase significantly until just after the first week of birth, indicating that differentiation of progenitor cells into hair cells can be delayed for several days after cell cycle exit (Burns and Stone, supra).

A combination of lineage tracing, onset of marker protein expression, and single-cell RNA-Seq trajectory analyses have revealed that differentiation of Type I and Type II hair cells in mice is temporally separated such that most, if not all, Type I hair cells differentiate embryonically whereas the majority of Type II differentiation occurs postnatally (McInturff et al., 2018. Biol. Open 7(11):bio038083; Warchol et al., 2019. J. Comp. Neurol. 527(11):1913-1928). At the molecular level, both Type I and II hair cells in striolar/central regions differ from their counterparts in extrastriolar/peripheral regions; however, the embryonic versus postnatal separation of Type I and Type II hair cell differentiation appears to hold regardless of region (Burns and Stone, supra).

The finding that Type I hair cells primarily differentiate embryonically came as somewhat of a surprise given that the hallmark electrophysiological characteristic of Type I hair cells, gKL, is not readily detectable in the majority of Type I hair cells until the first or second postnatal week (Rusch et al., 1998. J. Neurosci. 18(18):7487-7501). This suggests that there are two phases of Type I hair cell differentiation: an embryonic phase that might represent commitment to a Type I phenotype and a later, postnatal phase in which distinct Type I functionality emerges.

Thus, a clear picture is beginning to emerge in mice in which there are distinct and important differentiation events that span from mid-embryonic ages through at least the first two weeks after birth. The molecular cues that control these processes are also beginning to emerge. Recent work using conditional knockout mice has demonstrated that retinoic acid signaling controls the establishment of differences between striolar/central and extrastriolar/peripheral regions (Ono et al., 2020. Nat. Commun. 11(1):63).

In addition, the transcription factor Sox2 is expressed in supporting cells and in all Type II hair cells in mature vestibular organs, but not in Type I hair cells. Conditional knockout of Sox2 in developing hair cells at embryonic ages was reported to result in an increase in the number of Type I hair cells in just the striolar region of the utricle (crista and saccule not examined), presumably by reprogramming cells that were destined for a Type II fate into Type I hair cells (Lu et al., 2019. Neuroscience 422:146-160). Why Sox2 knockout would only increase the number of Type I hair cells in the striola, but not the extrastriola, is unclear since Sox2 is expressed in all Type II hair cells in both regions. One interpretation of this data is that Sox2 somehow only regulates differentiation of striolar/central Type I hair cells.

The inventors work reported herein demonstrates that the prior art results with Sox2 knockouts at the embryonic stage cannot predict the effect of inhibiting Sox2 in young and adult subjects in terms of converting mature vestibular Type II hair cells into Type I hair cells. Here we show that perturbation of pathways in developing ear organs in embryonic and early post-natal animals does not produce the same results in fully developed organs present at adulthood. Specifically, we have demonstrated that positive retinoic acid signaling is critical for Type I hair cell differentiation during normal embryonic and early development, but that manipulation of retinoic acid signaling in adult animals does not differentiate hair cells toward a Type I fate.

There is some spontaneous regeneration of hair cells that occurs in the vestibular system of mammals, but this mechanism appears to only produce Type II hair cells. In addition, the methods described to date that seek to stimulate regeneration of vestibular hair cells appear to only produce Type II hair cells. Type I hair cells are often more susceptible to damage than Type II hair cells, and in many vestibular pathologies Type II hair cells survive and persist in the sensory epithelia. Accordingly, it would be beneficial to differentiate regenerated hair cells into Type I hair cells (e.g., Type I hair cells that are able recruit new calyceal nerve endings). Converting pre-existing Type II hair cells into Type I hair cells could also have therapeutic benefit.

### Sox2

Sox2 is a transcription factor that is expressed in supporting cells and Type II hair cells of the vestibular system. In the cochlea, Sox2 expression is reported to be restricted to supporting cells. Developmentally, Sox2 is necessary for sensory epithelium development and supporting cell and hair cell formation. In addition, both overexpression and complete knockout of Sox2 prevent hair cell regeneration from supporting cell progenitors, whereas haploinsufficiency or partial knockdown enhances hair cell regeneration, suggesting that the level of Sox2 in supporting cells must be critically balanced to achieve regeneration. The mRNA and protein sequences for human and murine Sox2 are provided in Table 2, below.

TABLE 2
Sox2 mRNA and protein sequences

| SEQ ID | | |
|---|---|---|
| NO: | Description | Sequence |
| 1 | Human SOX2 | GATGGTTGTCTATTAACTTGTTCAAAAAAGTATCAGGAGTTGTCAAG |
| | mRNA sequence, | GCAGAGAAGAGAGTGTTTGCAAAAGGGGGAAAGTAGTTTGCTGCC |
| | NM_003106.4 | TCTTTAAGACTAGGACTGAGAGAAAGAAGAGGAGAGAGAAAGAAAG |
| | | GGAGAGAAGTTTGAGCCCCAGGCTTAAGCCTTTCCAAAAAATAATA |
| | | ATAACAATCATCGGCGGCGGCAGGATCGGCCAGAGGAGGAGGGA |
| | | AGCGCTTTTTTTGATCCTGATTCCAGTTTGCCTCTCTCTTTTTTTCCC |
| | | CCAAATTATTCTTCGCCTGATTTTCCTCGCGGAGCCCTGCGCTCCC |
| | | GACACCCCCGCCCGCCTCCCCTCCTCCTCTCCCCCCGCCCGCGG |
| | | GCCCCCCAAAGTCCCGGCCGGGCCGAGGGTCGGCGGCCGCCGG |
| | | CGGGCCGGGCCCGCGCACAGCGCCCGCATGTACAACATGATGGA |
| | | GACGGAGCTGAAGCCGCCGGGCCCGCAGCAAACTTCGGGGGGCG |
| | | GCGGCGGCAACTCCACCGCGGCGGCGGCCGGCGGCAACCAGAAA |
| | | AACAGCCCGGACCGCGTCAAGCGGCCCATGAATGCCTTCATGGTG |
| | | TGGTCCCGCGGGCAGCGGCGCAAGATGGCCCAGGAGAACCCCAA |
| | | GATGCACAACTCGGAGATCAGCAAGCGCCTGGGCGCCGAGTGGAA |
| | | ACTTTTGTCGGAGACGGAGAAGCGGCCGTTCATCGACGAGGCTAA |
| | | GCGGCTGCGAGCGCTGCACATGAAGGAGCACCCGGATTATAAATA |
| | | CCGGCCCCGGCGGAAAACCAAGACGCTCATGAAGAAGGATAAGTA |
| | | CACGCTGCCCGGCGGGCTGCTGGCCCCCGGCGGCAATAGCATGG |
| | | CGAGCGGGGTCGGGGTGGGCGCCGGCCTGGGCGCGGGCGTGAA |
| | | CCAGCGCATGGACAGTTACGCGCACATGAACGGCTGGAGCAACGG |
| | | CAGCTACAGCATGATGCAGGACCAGCTGGGCTACCCGCAGCACCC |
| | | GGGCCTCAATGCGCACGGCGCAGCGCAGATGCAGCCCATGCACC |
| | | GCTACGACGTGAGCGCCCTGCAGTACAACTCCATGACCAGCTCGC |
| | | AGACCTACATGAACGGCTCGCCCACCTACAGCATGTCCTACTCGCA |
| | | GCAGGGCACCCCTGGCATGGCTCTTGGCTCCATGGGTTCGGTGGT |
| | | CAAGTCCGAGGCCAGCTCCAGCCCCCCTGTGGTTACCTCTTCCTC |
| | | CCACTCCAGGGCGCCCTGCCAGGCCGGGGACCTCCGGGACATGA |
| | | TCAGCATGTATCTCCCCGGCGCCGAGGTGCCGGAACCCGCCGCC |
| | | CCCAGCAGACTTCACATGTCCCAGCACTACCAGAGCGGCCCGGTG |
| | | CCCGGCACGGCCATTAACGGCACACTGCCCCTCTCACACATGTGA |
| | | GGGCCGGACAGCGAACTGGAGGGGGGAGAAATTTTCAAAGAAAAA |
| | | CGAGGGAAATGGGAGGGGTGCAAAAGAGGAGAGTAAGAAACAGCA |
| | | TGGAGAAAACCCGGTACGCTCAAAAAGAAAAAGGAAAAAAAAAAAT |
| | | CCCATCACCCACAGCAAATGACAGCTGCAAAAGAGAACACCAATCC |
| | | CATCCACACTCACGCAAAAACCGCGATGCCGACAAGAAAACTTTTA |
| | | TGAGAGAGATCCTGGACTTCTTTTTGGGGGACTATTTTTGTACAGA |
| | | GAAAACCTGGGGAGGGTGGGGAGGGCGGGGGAATGGACCTTGTA |
| | | TAGATCTGGAGGAAAGAAAGCTACGAAAAACTTTTTAAAAGTTCTAG |
| | | TGGTACGGTAGGAGCTTTGCAGGAAGTTTGCAAAAGTCTTTACCAA |
| | | TAATATTTAGAGCTAGTCTCCAAGCGACGAAAAAAATGTTTTAATAT |
| | | TTGCAAGCAACTTTTGTACAGTATTTATCGAGATAAACATGGCAATC |
| | | AAAATGTCCATTGTTTATAAGCTGAGAATTTGCCAATATTTTTCAAG |
| | | GAGAGGCTTCTTGCTGAATTTTGATTCTGCAGCTGAAATTTAGGACA |
| | | GTTGCAAACGTGAAAAGAAGAAAATTATTCAAATTTGGACATTTTAA |
| | | TTGTTTAAAAATTGTACAAAAGGAAAAAATTAGAATAAGTACTGGCG |
| | | AACCATCTCTGTGGTCTTGTTTAAAAAGGGCAAAAGTTTTAGACTGT |
| | | ACTAAATTTTATAACTTACTGTTAAAAGCAAAAATGGCCATGCAGGT |
| | | TGACACCGTTGGTAATTTATAATAGCTTTTGTTCGATCCCAACTTTC |
| | | CATTTTGTTCAGATAAAAAAAACCATGAAATTACTGTGTTTGAAATAT |
| | | TTTCTTATGGTTTGTAATATTTCTGTAAATTTATTGTGATATTTTAAGG |
| | | TTTTCCCCCCTTTATTTTCCGTAGTTGTATTTTAAAAGATTCGGCTCT |
| | | GTATTATTTGAATCAGTCTGCCGAGAATCCATGTATATATTTGAACT |
| | | AATATCATCCTTATAACAGGTACATTTTCAACTTAAGTTTTTACTCCA |
| | | TTATGCACAGTTTGAGATAAATAAATTTTTGAAATATGGACACTGAA |
| | | A |
| 2 | Human Sox2 | MYNMMETELKPPGPQQTSGGGGGNSTAAAAGGNQKNSPDRVKRPM |
| | amino acid | NAFMVWSRGQRRKMAQENPKMHNSEISKRLGAEWKLLSETEKRPFID |
| | sequence, | EAKRLRALHMKEHPDYKYRPRRKTKTLMKKDKYTLPGGLLAPGGNSM |
| | Uniprot P48431 | ASGVGVGAGLGAGVNQRMDSYAHMNGWSNGSYSMMQDQLGYPQH |
| | | PGLNAHGAAQMQPMHRYDVSALQYNSMTSSQTYMNGSPTYSMSYS |
| | | QQGTPGMALGSMGSVVKSEASSSPPVVTSSSHSRAPCQAGDLRDMI |
| | | SMYLPGAEVPEPAAPSRLHMSQHYQSGPVPGTAINGTLPLSHM |
| 3 | Murine SOX2 | GGATGGTTGTCTATTAACTTGTTCAAAAAAGTATCAGGAGTTGTCAA |
| | mRNA sequence, | GGCAGAGAAGAGAGTGTTTGCAAAAAGGGAAAAGTACTTTGCTGCC |
| | NM_011443.4 | TCTTTAAGACTAGGGCTGGGAGAAAGAAGAGGAGAGAGAAAGAAA |
| | | GGAGAGAAGTTTGGAGCCCGAGGCTTAAGCCTTTCCAAAAACTAAT |
| | | CACAACAATCGCGGCGGCCCGAGGAGGAGAGCGCCTGTTTTTTCA |
| | | TCCCAATTGCACTTCGCCCGTCTCGAGCTCCGCTTCCCCCCAACTA |
| | | TTCTCCGCCAGATCTCCGCGCAGGGCCGTGCACGCCGAGGCCCC |
| | | CGCCCGCGGCCCCTGCATCCCGGCCCCCGAGCGCGGCCCCCACA |
| | | GTCCCGGCCGGGCCGAGGGTTGGCGGCCGCCGGCGGGCCGCGC |
| | | CCGCCCAGCGCCCGCATGTATAACATGATGGAGACGGAGCTGAAG |
| | | CCGCCGGGCCCGCAGCAAGCTTCGGGGGGCGGCGGCGGAGGAG |
| | | GCAACGCCACGGCGGCGGCGACCGGCGGCAACCAGAAGAACAGC |
| | | CCGGACCGCGTCAAGAGGCCCATGAACGCCTTCATGGTATGGTCC |
| | | CGGGGGCAGCGGCGTAAGATGGCCCAGGAGAACCCCAAGATGCA |
| | | CAACTCGGAGATCAGCAAGCGCCTGGGCGCGGAGTGGAAACIIII |
| | | GTCCGAGACCGAGAAGCGGCCGTTCATCGACGAGGCCAAGCGGC |
| | | TGCGCGCTCTGCACATGAAGGAGCACCCGGATTATAAATACCGGC |
| | | CGCGGCGGAAAACCAAGACGCTCATGAAGAAGGATAAGTACACGC |
| | | TTCCCGGAGGCTTGCTGGCCCCCGGCGGGAACAGCATGGCGAGC |
| | | GGGGTTGGGGTGGGCGCCGGCCTGGGTGCGGGCGTGAACCAGC |
| | | GCATGGACAGCTACGCGCACATGAACGGCTGGAGCAACGGCAGCT |
| | | ACAGCATGATGCAGGAGCAGCTGGGCTACCCGCAGCACCCGGGC |
| | | CTCAACGCTCACGGCGCGGCACAGATGCAACCGATGCACCGCTAC |
| | | GACGTCAGCGCCCTGCAGTACAACTCCATGACCAGCTCGCAGACC |
| | | TACATGAACGGCTCGCCCACCTACAGCATGTCCTACTCGCAGCAG |
| | | GGCACCCCCGGTATGGCGCTGGGCTCCATGGGCTCTGTGGTCAAG |
| | | TCCGAGGCCAGCTCCAGCCCCCCCGTGGTTACCTCTTCCTCCCAC |
| | | TCCAGGGCGCCCTGCCAGGCCGGGGACCTCCGGGACATGATCAG |
| | | CATGTACCTCCCCGGCGCCGAGGTGCCGGAGCCCGCTGCGCCCA |
| | | GTAGACTGCACATGGCCCAGCACTACCAGAGCGGCCCGGTGCCC |
| | | GGCACGGCCATTAACGGCACACTGCCCCTGTCGCACATGTGAGGG |
| | | CTGGACTGCGAACTGGAGAAGGGGAGAGATTTTCAAAGAGATACA |
| | | AGGGAATTGGGAGGGGTGCAAAAAGAGGAGAGTAGGAAAAATCTG |
| | | ATAATGCTCAAAAGGAAAAAAAATCTCCGCAGCGAAACGACAGCTG |
| | | CGGAAAAAAACCACCAATCCCATCCAAATTAACGCAAAAACCGTGA |
| | | TGCCGACTAGAAAACTTTTATGAGAGATCTTGGGACTTCTTTTTGGG |
| | | GGACTATTTTTGTACAGAGAAAACCTGAGGGCGGCGGGGAGGGCG |
| | | GGGGAATCGGACCATGTATAGATCTGGAGGAAAAAAACTACGCAAA |
| | | ACTTTTTTTTAAAGTTCTAGTGGTACGTTAGGCGCTTCGCAGGGAGT |
| | | TCGCAAAAGTCTTTACCAGTAATATTTAGAGCTAGACTCCGGGCGA |
| | | TGAAAAAAAAGTTTTAATATTTGCAAGCAACTTTTGTACAGTATTTAT |
| | | CGAGATAAACATGGCAATCAAATGTCCATTGTTTATAAGCTGAGAAT |
| | | TTGCCAATATTTTTCGAGGAAAGGGTTCTTGCTGGGTTTTGATTCTG |
| | | CAGCTTAAATTTAGGACCGTTACAAACAAGGAAGGAGTTTATTCGG |
| | | ATTTGAACATTTTAGTTTT1AAAATTGTACAAAAGGAAAACATGAGAG |
| | | CAAGTACTGGCAAGACCGTTTTCGTGGTCTTGTTTAAGGCAAACGT |
| | | TCTAGATTGTACTAAATTTTTAACTTACTGTTAAAGGCAAAAAAAAAA |
| | | TGTCCATGCAGGTTGATATCGTTGGTAATTTATAATAGCTTTTGTTC |
| | | AATCCTACCCTTTCATTTTGTTCACATAAAAAATATGGAATTACTGTG |
| | | TTTGAAATATTTTCTTATGGTTTGTAATATTTCTGTAAATTGTGATATT |
| | | TTAAGGTTTTTCCCCCCTTTTATTTTCCGTAGTTGTATTTTAAAAGAT |
| | | TCGGCTCTGTTATTGGAATCAGGCTGCCGAGAATCCATGTATATATT |
| | | TGAACTAATACCATCCTTATAACAGCTACATTTTCAACTTAAGTTTTT |
| | | ACTCCATTATGCACAGTTTGAGATAAATAAATTTTTGAAATATGGAC |
| | | ACTGAAAAAAAAAAAAAAAA |
| 4 | Murine Sox2 | MYNMMETELKPPGPQQASGGGGGGGNATAAATGGNQKNSPDRVKR |
| | amino acid | PMNAFMVWSRGQRRKMAQENPKMHNSEISKRLGAEWKLLSETEKRP |
| | sequence, | FIDEAKRLRALHMKEHPDYKYRPRRKTKTLMKKDKYTLPGGLLAPGGN |
| | Uniprot P48432 | SMASGVGVGAGLGGGLNQRMDSYAHMNGWSNGSYSMMQEQLGYP |
| | | QHPGLNAHGAAQMQPMHRYVVSALQYNSMTSSQTYMNGSPTYSMS |
| | | YSQQGTPGMALGSMGSVVKSEASSSPPVVTSSSHSRAPCQAGDLRD |
| | | MISMYLPGAEVPEPAAPSRLHMAQHYQSGPVPGTAKYGTLPLSHM |

The present invention provides methods of generating Type I vestibular hair cells by reducing Sox2 activity or expression in Type II hair cells, regenerated or regenerating hair cells, or supporting cells that are then regenerated into hair cells. These methods are based on the use of Sox2 inhibitors and can utilize hair cell, Type II hair cell, or supporting cell promoters to target Sox2 inhibitors specifically to hair cells, Type II hair cells, or vestibular supporting cells. In addition, these methods can be performed in combination with methods that promote hair cell regeneration (e.g., Atoh1 overexpression or Notch inhibition) to provide regenerated hair cells for conversion into Type I hair cells. Increasing the number of Type I vestibular hair cells can be used to treat subjects having or at risk of developing vestibular dysfunction, such as loss of balance, dizziness, vertigo, bilateral vestibulopathy (bilateral vestibular hypofunction), oscillopsia, or a balance disorder.

### Sox2 Inhibitors

A Sox2 inhibitor for use in the methods and compositions described herein may inhibit Sox2 by reducing Sox2 activity or expression. The Sox2 inhibitor may be a nucleic acid molecule (e.g., an RNA or DNA molecule), a protein, or a component of a gene editing system. In some embodiments, the Sox2 inhibitor reduces Sox2 activity or expression in Type II hair cells in the vestibular system. A Sox2 inhibitor can also be used to reduce Sox2 activity or expression in hair cells that have been produced by regeneration (regenerated hair cells) or in hair cells that are currently undergoing regeneration (regenerating hair cells). In some embodiments, the Sox2 inhibitor is delivered to a supporting cell before the supporting cell is made to regenerate into a hair cell. Exemplary Sox2 inhibitors are described herein below.

Inhibitory RNA

In some embodiments, the Sox2 inhibitor is an inhibitory RNA molecule, e.g., that acts by way of the RNA interference (RNAi) pathway. An inhibitory RNA molecule can decrease the expression level (e.g., protein level or mRNA level) of Sox2. Inhibitory RNA molecules include short interfering RNA (siRNA) molecules, short hairpin RNA (shRNA) molecules, and/or microRNA (miRNA) molecules that target full-length Sox2. An siRNA is a double-stranded RNA molecule that typically has a length of about 19-25 base pairs. An shRNA is an RNA molecule containing a hairpin turn that decreases expression of target genes via RNAi. shRNAs can be delivered to cells in the form of plasmids, e.g., viral or bacterial vectors, such as adeno-associated virus vectors (AAV vectors), e.g., by transfection, electroporation, or transduction. An shRNA can also be embedded into the backbone of an miRNA (e.g., miRNA-30 or mir-E, e.g., to produce an shRNA-mir), as described in Silva et al., Nature Genetics 37:1281-1288 (2005) and Fellmann et al., Cell Reports 5:1704-1713 (2013), to achieve highly efficient target gene knockdown. Exemplary shRNA and siRNA target sequences are provided in Tables 3 and 4, below. Sequences for plasmids containing exemplary shRNAs that are embedded in miRNA backbones are provided in Table 5, below. Exemplary siRNA sequences are provided in Table 6, below.

TABLE 3
Human Sox2 shRNA and siRNA targets

| SEQ ID NO: | Target sequence |
|---|---|
| 5 | CTGCCGAGAATCCATGTATAT |
| 6 | GTACAGTATTTATCGAGATAA |
| 7 | AGGAGCACCCGGATTATAAAT |
| 8 | TGGACAGTTACGCGCACATGA |
| 9 | TCCCATCACCCACAGCAAATG |
| 10 | CGAGATAAACATGGCAATCAA |
| 11 | CGCTCATGAAGAAGGATAAGT |
| 12 | CAGCTCGCAGACCTACATGAA |
| 13 | CAACGGCAGCTACAGCATGAT |
| 14 | CCACCTACAGCATGTCCTACT |
| 15 | CCCTGCAGTACAACTCCATGA |
| 16 | ACATGTCCCAGCACTACCAGA |
| 17 | GCACATGAACGGCTGGAGCAA |
| 18 | GCCCACCTACAGCATGTCCTA |
| 19 | GAAGAAGGATAAGTACACGCT |
| 25 | CCAGTAATATTTAGAGCTA |
| 26 | TTGTGATATTTTAAGGTTT |
| 27 | CTTATGGTTTGTAATATTT |
| 28 | TTGATTGCCATGTTTATCTCGA |
| 29 | TTATCTCGATAAATACTGTACA |

TABLE 4
Mouse Sox2 shRNA and siRNA targets

| SEQ ID NO: | Target sequence |
|---|---|
| 5 | CTGCCGAGAATCCATGTATAT |
| 6 | GTACAGTATTTATCGAGATAA |
| 7 | AGGAGCACCCGGATTATAAAT |
| 10 | CGAGATAAACATGGCAATCAA |
| 11 | CGCTCATGAAGAAGGATAAGT |
| 12 | CAGCTCGCAGACCTACATGAA |
| 13 | CAACGGCAGCTACAGCATGAT |
| 14 | CCACCTACAGCATGTCCTACT |
| 15 | CCCTGCAGTACAACTCCATGA |
| 17 | GCACATGAACGGCTGGAGCAA |
| 18 | GCCCACCTACAGCATGTCCTA |
| 19 | GAAGAAGGATAAGTACACGCT |
| 20 | ACCAATCCCATCCAAATTAAC |
| 21 | CAAAGAGATACAAGGGAATTG |
| 22 | TGCGCCCAGTAGACTGCACAT |
| 23 | CGCGGCACAGATGCAACCGAT |

TABLE 5
Exemplary plasmid sequences containing shRNAs in an miRNA scaffold

| SEQ ID NO: | Plasmid sequence |
|---|---|
| 30 | ccttaattaggctgcgcgctcgctcgctcactgaggccgcccgggcaaagcccgggcgtcgggcgacctttggtcg |
| (P797) | cccggcctcagtgagcgagcgagcgcgcagagagggagtggccaactccatcactaggggttccttgtagttaat |
| 5′-mir 30 | gattaacccgccatgctacttatctacgtagccatgctctaggaagatcggaattcgcccttaagctagcggcgcgcc |
| sequence at | accggtgcgatcgccgttacataacttacggtaaatggcccgcctggctgaccgcccaacgacccccgcccattga |
| positions | cgtcaataatgacgtatgttcccatagtaacgccaatagggactttccattgacgtcaatgggtggagtatttacggta |
| 2109- | aactgcccacttggcagtacatcaagtgtatcatatgccaagtacgccccctattgacgtcaatgacggtaaatggcc |
| 2233 | cgcctggcattatgcccagtacatgaccttatgggactttcctacttggcagtacatctacgtattagtcatcgctattacc |
| shRNA_Sox2_2 | atggtgatgcggttttggcagtacatcaatgggcgtggatagcggtttgactcacggggatttccaagtctccacccca |
| sequence at | ttgacgtcaatgggagtttgttttggcaccaaaatcaacgggactttccaaaatgtcgtaacaactccgccccattgac |
| positions | gcaaatgggcggtaggcgtgtacggtgggaggtctatataagcagagctggtttagtgaaccgtcagatcctgcag |
| 2234- | aagttggtcgtgaggcactgggcaggtaagtatcaaggttacaagacaggtttaaggagaccaatagaaactggg |
| 2296 | cttgtcgagacagagaagactcttgcgtttctgataggcacctattggtcttactgacatccactttgcctttctctccaca |
| 3′-mir 30 | ggtgtccaggcggccgcgccaccatgccagagccagcgaagtctgctcccgccccgaaaaagggctccaagaa |
| sequence | ggcggtgactaaggcgcagaagaaaggcggcaagaagcgcaagcgcagccgcaaggagagctattccatcta |
| at | tgtgtacaaggttctgaagcaggtccaccctgacaccggcatttcgtccaaggccatgggcatcatgaattcgtttgtg |
| positions | aacgacattttcgagcgcatcgcaggtgaggcttcccgcctggcgcattacaacaagcgctcgaccatcacctcca |
| 2297- | gggagatccagacggccgtgcgcctgctgctgcctggggagttggccaagcacgccgtgtccgagggtactaag |
| 2426 | gccatcaccaagtacaccagcgctaaggatccaccggtcgccaccatggtgagcaagggcgaggagctgttcac |
| | cggggtggtgcccatcctggtcgagctggacggcgacgtaaacggccacaagttcagcgtgtccggcgagggcg |
| | agggcgatgccacctacggcaagctgaccctgaagttcatctgcaccaccggcaagctgcccgtgccctggccca |
| | ccctcgtgaccaccctgacctacggcgtgcagtgcttcagccgctaccccgaccacatgaagcagcacgacttcttc |
| | aagtccgccatgcccgaaggctacgtccaggagcgcaccatcttcttcaaggacgacggcaactacaagacccg |
| | cgccgaggtgaagttcgagggcgacaccctggtgaaccgcatcgagctgaagggcatcgacttcaaggaggac |
| | ggcaacatcctggggcacaagctggagtacaactacaacagccacaacgtctatatcatggccgacaagcaga |
| | agaacggcatcaaggtgaacttcaagatccgccacaacatcgaggacggcagcgtgcagctcgccgaccacta |
| | ccagcagaacacccccatcggcgacggccccgtgctgctgcccgacaaccactacctgagcacccagtccgcc |
| | ctgagcaaagaccccaacgagaagcgcgatcacatggtcctgctggagttcgtgaccgccgccgggatcactctc |
| | ggcatggacgagctgtacaagtaataagcttctcgactagggataacagggtaattgtttgaatgaggcttcagtactt |
| | tacagaatcgttgcctgcacatcttggaaacacttgctgggattacttcttcaggttaacccaacagaaggctcgaga |
| | aggtatattgctgttgAcagtgAgcgCcgagataaacatggcaatcaatagtgaagccacagatgtattgattgcc |
| | atgtttatctcgatgcCtactgCctcgcaattgaaggggctactttaggagcaattatcttgtttactaaaactgaatacc |
| | ttgctatctctttgatacatttttacaaagctgaattaaaatggtataaattaaatcacttttataaattaaatcactttttta |
| | cgcgtggatccaatcaacctctggattacaaaatttgtgaaagattgactggtattcttaactatgttgctccttttacgctat |
| | gtggatacgctgctttaatgcctttgtatcatgctattgcttcccgtatggctttcattttctcctccttgtataaatcctggt |
| | tgctgtctctttatgaggagttgtggcccgttgtcaggcaacgtggcgtggtgtgcactgtgtttgctgacgcaacccccactg |
| | gttggggcattgccaccacctgtcagctcctttccgggactttcgctttccccctccctattgccacggcggaactcatc |
| | gccgcctgccttgcccgctgctggacaggggctcggctgttgggcactgacaattccgtggtgttgtcggggaaatca |
| | tcgtcctttccttggctgctcgcctgtgttgccacctggattctgcgcgggacgtccttctgctacgtcccttcggccctca |
| | atccagcggaccttccttcccgcggcctgctgccggctctgcggcctcttccgcgtcttcgagatctgcctcgactgtgc |
| | cttctagttgccagccatctgttgtttgcccctcccccgtgccttccttgaccctggaaggtgccactcccactgtcctttcc |
| | taataaaatgaggaaattgcatcgcattgtctgagtaggtgtcattctattctggggggtggggtggggcaggacagc |
| | aagggggaggattgggaagacaatagcaggcatgctggggagagctcttaagggcgaattcccgataaggatct |
| | tcctagagcatggctacgtagataagtagcatggcgggttaatcattaactacaaggaacccctagtgatggagttg |
| | gccactccctctctgcgcgctcgctcgctcactgaggccgggcgaccaaaggtcgcccgacgcccgggctttgccc |
| | gggcggcctcagtgagcgagcgagcgcgcagccttaattaacctaattcactggccgtcgttttacaacgtcgtgact |
| | gggaaaaccctggcgttacccaacttaatcgccttgcagcacatccccctttcgccagctggcgtaatagcgaaga |
| | ggcccgcaccgatcgcccttcccaacagttgcgcagcctgaatggcgaatgggacgcgccctgtagcggcgcatt |
| | aagcgcggcgggtgtggtggttacgcgcagcgtgaccgctacacttgccagcgccctagcgcccgctcctttcgcttt |
| | cttcccttcctttctcgccacgttcgccggctttccccgtcaagctctaaatcgggggctccctttagggttccgatttagtg |
| | ctttacggcacctcgaccccaaaaaacttgattagggtgatggttcacgtagtgggccatcgccctgatagacggtttt |
| | tcgccctttgacgttggagtccacgttctttaatagtggactcttgttccaaactggaacaacactcaaccctatctcggt |
| | ctattcttttgatttataagggattttgccgatttcggcctattggttaaaaaatgagctgatttaacaaaaatttaacgcga |
| | attttaacaaaatattaacgcttacaatttaggtggcacttttcggggaaatgtgcgcggaacccctatttgtttatttttcta |
| | aatacattcaaatatgtatccgctcatgagacaataaccctgataaatgcttcaataatattgaaaaaggaagagtat |
| | gagccatattcaacgggaaacgtcgaggccgcgattaaattccaacatggatgctgatttatatgggtataaatggg |
| | ctcgcgataatgtcgggcaatcaggtgcgacaatctatcgcttgtatgggaagcccgatgcgccagagttgtttctga |
| | aacatggcaaaggtagcgttgccaatgatgttacagatgagatggtcagactaaactggctgacggaatttatgcct |
| | cttccgaccatcaagcattttatccgtactcctgatgatgcatggttactcaccactgcgatccccggaaaaacagcat |
| | tccaggtattagaagaatatcctgattcaggtgaaaatattgttgatgcgctggcagtgttcctgcgccggttgcattcg |
| | attcctgtttgtaattgtccttttaacagcgatcgcgtatttcgtcttgctcaggcgcaatcacgaatgaataacggtttggtt |
| | gatgcgagtgattttgatgacgagcgtaatggctggcctgttgaacaagtctggaaagaaatgcataaacttttgccat |
| | tctcaccggattcagtcgtcactcatggtgatttctcacttgataaccttatttttgacgaggggaaattaataggttgtatt |
| | gatgttggacgagtcggaatcgcagaccgataccaggatcttgccatcctatggaactgcctcggtgagttttctcctt |
| | cattacagaaacggctttttcaaaaatatggtattgataatcctgatatgaataaattgcagtttcatttgatgctcgatga |
| | gtttttctaactgtcagaccaagtttactcatatatactttagattgatttaaaacttcatttttaatttaaaaggatctaggtg |
| | aagatcctttttgataatctcatgaccaaaatcccttaacgtgagttttcgttccactgagcgtcagaccccgtagaaaa |
| | gatcaaaggatcttcttgagatcctttttttctgcgcgtaatctgctgcttgcaaacaaaaaaaccaccgctaccagcg |
| | gtggtttgtttgccggatcaagagctaccaactctttttccgaaggtaactggcttcagcagagcgcagataccaaata |
| | ctgttcttctagtgtagccgtagttaggccaccacttcaagaactctgtagcaccgcctacatacctcgctctgctaatcc |
| | tgttaccagtggctgctgccagtggcgataagtcgtgtcttaccgggttggactcaagacgatagttaccggataagg |
| | cgcagcggtcgggctgaacggggggttcgtgcacacagcccagcttggagcgaacgacctacaccgaactgag |
| | atacctacagcgtgagctatgagaaagcgccacgcttcccgaagggagaaaggcggacaggtatccggtaagc |
| | ggcagggtcggaacaggagagcgcacgagggagcttccagggggaaacgcctggtatctttatagtcctgtcgg |
| | gtttcgccacctctgacttgagcgtcgatttttgtgatgctcgtcaggggggcggagcctatggaaaaacgccagcaa |
| | cgcggcctttttacggttcctggccttttgctggccttttgctcacatgttctttcctgcgttatcccctgattctgtggataa |
| | ccgtattaccgcctttgagtgagctgataccgctcgccgcagccgaacgaccgagcgcagcgagtcagtgagcgagg |
| | aagcggaagagcgcccaatacgcaaaccgcctctccccgcgcgttggccgattcattaatgcagctggcacgac |
| | aggtttcccgactggaaagcgggcagtgagcgcaacgcaattaatgtgagttagctcactcattaggcaccccagg |
| | ctttacactttatgcttccggctcgtatgttgtgtggaattgtgagcggataacaatttcacacaggaaacagctatgacc |
| | atgattacgccagatttaattaagg |
| 31 | ccttaattaggctgcgcgctcgctcgctcactgaggccgcccgggcaaagcccgggcgtcgggcgacctttggtcg |
| (P900) | cccggcctcagtgagcgagcgagcgcgcagagagggagtggccaactccatcactaggggttccttgtagttaat |
| 5′-mirE | gattaacccgccatgctacttatctacgtagccatgctctaggaagatcggaattcgcccttaagctagcggcgcgcc |
| sequence at | accggtgcgatcgccgttacataacttacggtaaatggcccgcctggctgaccgcccaacgacccccgcccattga |
| positions | cgtcaataatgacgtatgttcccatagtaacgccaatagggactttccattgacgtcaatgggtggagtatttacggta |
| 2109- | aactgcccacttggcagtacatcaagtgtatcatatgccaagtacgccccctattgacgtcaatgacggtaaatggcc |
| 2233 | cgcctggcattatgcccagtacatgaccttatgggactttcctacttggcagtacatctacgtattagtcatcgctattacc |
| shRNA_Sox2_2 | atggtgatgcggttttggcagtacatcaatgggcgtggatagcggtttgactcacggggatttccaagtctccacccca |
| sequence at | ttgacgtcaatgggagtttgttttggcaccaaaatcaacgggactttccaaaatgtcgtaacaactccgccccattgac |
| positions | gcaaatgggcggtaggcgtgtacggtgggaggtctatataagcagagctggtttagtgaaccgtcagatcctgcag |
| 2234- | aagttggtcgtgaggcactgggcaggtaagtatcaaggttacaagacaggtttaaggagaccaatagaaactggg |
| 2296 | cttgtcgagacagagaagactcttgcgtttctgataggcacctattggtcttactgacatccactttgcctttctctccaca |
| 3′-mirE | ggtgtccaggcggccgcgccaccatgccagagccagcgaagtctgctcccgccccgaaaaagggctccaagaa |
| sequence at | ggcggtgactaaggcgcagaagaaaggcggcaagaagcgcaagcgcagccgcaaggagagctattccatcta |
| positions | tgtgtacaaggttctgaagcaggtccaccctgacaccggcatttcgtccaaggccatgggcatcatgaattcgtttgtg |
| 2297- | aacgacattttcgagcgcatcgcaggtgaggcttcccgcctggcgcattacaacaagcgctcgaccatcacctcca |
| 2408 | gggagatccagacggccgtgcgcctgctgctgcctggggagttggccaagcacgccgtgtccgagggtactaag |
| | gccatcaccaagtacaccagcgctaaggatccaccggtcgccaccatggtgagcaagggcgaggagctgttcac |
| | cggggtggtgcccatcctggtcgagctggacggcgacgtaaacggccacaagttcagcgtgtccggcgagggcg |
| | agggcgatgccacctacggcaagctgaccctgaagttcatctgcaccaccggcaagctgcccgtgccctggccca |
| | ccctcgtgaccaccctgacctacggcgtgcagtgcttcagccgctaccccgaccacatgaagcagcacgacttcttc |
| | aagtccgccatgcccgaaggctacgtccaggagcgcaccatcttcttcaaggacgacggcaactacaagacccg |
| | cgccgaggtgaagttcgagggcgacaccctggtgaaccgcatcgagctgaagggcatcgacttcaaggaggac |
| | ggcaacatcctggggcacaagctggagtacaactacaacagccacaacgtctatatcatggccgacaagcaga |
| | agaacggcatcaaggtgaacttcaagatccgccacaacatcgaggacggcagcgtgcagctcgccgaccacta |
| | ccagcagaacacccccatcggcgacggccccgtgctgctgcccgacaaccactacctgagcacccagtccgcc |
| | ctgagcaaagaccccaacgagaagcgcgatcacatggtcctgctggagttcgtgaccgccgccgggatcactctc |
| | ggcatggacgagctgtacaagtaataagcttctcgactagggataacagggtaattgtttgaatgaggcttcagtactt |
| | tacagaatcgttgcctgcacatcttggaaacacttgctgggattacttcgacttcttaacccaacagaaggctcgaga |
| | aggtatattgctgttgacagtgagcgccgagataaacatggcaatcaatagtgaagccacagatgtattgattgccat |
| | gtttatctcgatgcctactgcctcggacttcaaggggctagaattcgagcaattatcttgtttactaaaactgaataccttg |
| | ctatctctttgatacatttttacaaagctgaattaaaatggtataaattaaatcacttttttcaattgacgcgtaattctaccg |
| | gatccaatcaacctctggattacaaaatttgtgaaagattgactggtattcttaactatgttgctccttttacgctatgtgga |
| | tacgctgctttaatgcctttgtatcatgctattgcttcccgtatggctttcattttctcctccttgtataaatcctggttgctg |
| | tctctttatgaggagttgtggcccgttgtcaggcaacgtggcgtggtgtgcactgtgtttgctgacgcaacccccactggttgg |
| | ggcattgccaccacctgtcagctcctttccgggactttcgctttccccctccctattgccacggcggaactcatcgccgc |
| | ctgccttgcccgctgctggacaggggctcggctgttgggcactgacaattccgtggtgttgtcggggaaatcatcgtcc |
| | tttccttggctgctcgcctgtgttgccacctggattctgcgcgggacgtccttctgctacgtcccttcggccctcaatccag |
| | cggaccttccttcccgcggcctgctgccggctctgcggcctcttccgcgtcttcgagatctgcctcgactgtgccttctag |
| | ttgccagccatctgttgtttgcccctcccccgtgccttccttgaccctggaaggtgccactcccactgtcctttcctaataa |
| | aatgaggaaattgcatcgcattgtctgagtaggtgtcattctattctggggggtggggtggggcaggacagcaaggg |
| | ggaggattgggaagacaatagcaggcatgctggggagagctcttaagggcgaattcccgataaggatcttcctag |
| | agcatggctacgtagataagtagcatggcgggttaatcattaactacaaggaacccctagtgatggagttggccact |
| | ccctctctgcgcgctcgctcgctcactgaggccgggcgaccaaaggtcgcccgacgcccgggctttgcccgggcg |
| | gcctcagtgagcgagcgagcgcgcagccttaattaacctaattcactggccgtcgttttacaacgtcgtgactggga |
| | aaaccctggcgttacccaacttaatcgccttgcagcacatccccctttcgccagctggcgtaatagcgaagaggccc |
| | gcaccgatcgcccttcccaacagttgcgcagcctgaatggcgaatgggacgcgccctgtagcggcgcattaagcg |
| | cggcgggtgtggtggttacgcgcagcgtgaccgctacacttgccagcgccctagcgcccgctcctttcgctttcttccc |
| | ttcctttctcgccacgttcgccggctttccccgtcaagctctaaatcgggggctccctttagggttccgatttagtgctttac |
| | ggcacctcgaccccaaaaaacttgattagggtgatggttcacgtagtgggccatcgccctgatagacggtttttcgcc |
| | ctttgacgttggagtccacgttctttaatagtggactcttgttccaaactggaacaacactcaaccctatctcggtctattct |
| | tttgatttataagggattttgccgatttcggcctattggttaaaaaatgagctgatttaacaaaaatttaacgcgaattttaa |
| | caaaatattaacgcttacaatttaggtggcacttttcggggaaatgtgcgcggaacccctatttgtttatttttctaaatac |
| | attcaaatatgtatccgctcatgagacaataaccctgataaatgcttcaataatattgaaaaaggaagagtatgagcc |
| | atattcaacgggaaacgtcgaggccgcgattaaattccaacatggatgctgatttatatgggtataaatgggctcgcg |
| | ataatgtcgggcaatcaggtgcgacaatctatcgcttgtatgggaagcccgatgcgccagagttgtttctgaaacatg |
| | gcaaaggtagcgttgccaatgatgttacagatgagatggtcagactaaactggctgacggaatttatgcctcttccga |
| | ccatcaagcattttatccgtactcctgatgatgcatggttactcaccactgcgatccccggaaaaacagcattccaggt |
| | attagaagaatatcctgattcaggtgaaaatattgttgatgcgctggcagtgttcctgcgccggttgcattcgattcctgtt |
| | tgtaattgtccttttaacagcgatcgcgtatttcgtcttgctcaggcgcaatcacgaatgaataacggtttggttgatgcg |
| | agtgattttgatgacgagcgtaatggctggcctgttgaacaagtctggaaagaaatgcataaacttttgccattctcac |
| | cggattcagtcgtcactcatggtgatttctcacttgataaccttatttttgacgaggggaaattaataggttgtattgatgttg |
| | gacgagtcggaatcgcagaccgataccaggatcttgccatcctatggaactgcctcggtgagttttctccttcattaca |
| | gaaacggctttttcaaaaatatggtattgataatcctgatatgaataaattgcagtttcatttgatgctcgatgagtttttcta |
| | actgtcagaccaagtttactcatatatactttagattgatttaaaacttcatttttaatttaaaaggatctaggtgaagatcct |
| | ttttgataatctcatgaccaaaatcccttaacgtgagttttcgttccactgagcgtcagaccccgtagaaaagatcaaa |
| | ggatcttcttgagatcctttttttctgcgcgtaatctgctgcttgcaaacaaaaaaaccaccgctaccagcggtggtttgtt |
| | tgccggatcaagagctaccaactctttttccgaaggtaactggcttcagcagagcgcagataccaaatactgttcttct |
| | agtgtagccgtagttaggccaccacttcaagaactctgtagcaccgcctacatacctcgctctgctaatcctgttacca |
| | gtggctgctgccagtggcgataagtcgtgtcttaccgggttggactcaagacgatagttaccggataaggcgcagcg |
| | gtcgggctgaacggggggttcgtgcacacagcccagcttggagcgaacgacctacaccgaactgagatacctac |
| | agcgtgagctatgagaaagcgccacgcttcccgaagggagaaaggcggacaggtatccggtaagcggcaggg |
| | tcggaacaggagagcgcacgagggagcttccagggggaaacgcctggtatctttatagtcctgtcgggtttcgcca |
| | cctctgacttgagcgtcgatttttgtgatgctcgtcaggggggcggagcctatggaaaaacgccagcaacgcggcct |
| | ttttacggttcctggccttttgctggccttttgctcacatgttctttcctgcgttatcccctgattctgtggataaccgtattac |
| | cgcctttgagtgagctgataccgctcgccgcagccgaacgaccgagcgcagcgagtcagtgagcgaggaagcgga |
| | agagcgcccaatacgcaaaccgcctctccccgcgcgttggccgattcattaatgcagctggcacgacaggtttccc |
| | gactggaaagcgggcagtgagcgcaacgcaattaatgtgagttagctcactcattaggcaccccaggctttacactt |
| | tatgcttccggctcgtatgttgtgtggaattgtgagcggataacaatttcacacaggaaacagctatgaccatgattac |
| | gccagatttaattaagg |
| 32 | ccttaattaggctgcgcgctcgctcgctcactgaggccgcccgggcaaagcccgggcgtcgggcgacctttggtcg |
| (P799) | cccggcctcagtgagcgagcgagcgcgcagagagggagtggccaactccatcactaggggttccttgtagttaat |
| 5′-mir30 | gattaacccgccatgctacttatctacgtagccatgctctaggaagatcggaattcgcccttaagctagcggcgcgcc |
| sequence at | accggtgcgatcgccgttacataacttacggtaaatggcccgcctggctgaccgcccaacgacccccgcccattga |
| positions | cgtcaataatgacgtatgttcccatagtaacgccaatagggactttccattgacgtcaatgggtggagtatttacggta |
| 2109- | aactgcccacttggcagtacatcaagtgtatcatatgccaagtacgccccctattgacgtcaatgacggtaaatggcc |
| 2233 | cgcctggcattatgcccagtacatgaccttatgggactttcctacttggcagtacatctacgtattagtcatcgctattacc |
| shRNA_Sox2_4 | atggtgatgcggttttggcagtacatcaatgggcgtggatagcggtttgactcacggggatttccaagtctccacccca |
| sequence at | ttgacgtcaatgggagtttgttttggcaccaaaatcaacgggactttccaaaatgtcgtaacaactccgccccattgac |
| positions | gcaaatgggcggtaggcgtgtacggtgggaggtctatataagcagagctggtttagtgaaccgtcagatcctgcag |
| 2234- | aagttggtcgtgaggcactgggcaggtaagtatcaaggttacaagacaggtttaaggagaccaatagaaactggg |
| 2296 | cttgtcgagacagagaagactcttgcgtttctgataggcacctattggtcttactgacatccactttgcctttctctccaca |
| 3′-mir 30 | ggtgtccaggcggccgcgccaccatgccagagccagcgaagtctgctcccgccccgaaaaagggctccaagaa |
| sequence at | ggcggtgactaaggcgcagaagaaaggcggcaagaagcgcaagcgcagccgcaaggagagctattccatcta |
| positions | tgtgtacaaggttctgaagcaggtccaccctgacaccggcatttcgtccaaggccatgggcatcatgaattcgtttgtg |
| 2297- | aacgacattttcgagcgcatcgcaggtgaggcttcccgcctggcgcattacaacaagcgctcgaccatcacctcca |
| 2426 | gggagatccagacggccgtgcgcctgctgctgcctggggagttggccaagcacgccgtgtccgagggtactaag |
| | gccatcaccaagtacaccagcgctaaggatccaccggtcgccaccatggtgagcaagggcgaggagctgttcac |
| | cggggtggtgcccatcctggtcgagctggacggcgacgtaaacggccacaagttcagcgtgtccggcgagggcg |
| | agggcgatgccacctacggcaagctgaccctgaagttcatctgcaccaccggcaagctgcccgtgccctggccca |
| | ccctcgtgaccaccctgacctacggcgtgcagtgcttcagccgctaccccgaccacatgaagcagcacgacttcttc |
| | aagtccgccatgcccgaaggctacgtccaggagcgcaccatcttcttcaaggacgacggcaactacaagacccg |
| | cgccgaggtgaagttcgagggcgacaccctggtgaaccgcatcgagctgaagggcatcgacttcaaggaggac |
| | ggcaacatcctggggcacaagctggagtacaactacaacagccacaacgtctatatcatggccgacaagcaga |
| | agaacggcatcaaggtgaacttcaagatccgccacaacatcgaggacggcagcgtgcagctcgccgaccacta |
| | ccagcagaacacccccatcggcgacggccccgtgctgctgcccgacaaccactacctgagcacccagtccgcc |
| | ctgagcaaagaccccaacgagaagcgcgatcacatggtcctgctggagttcgtgaccgccgccgggatcactctc |
| | ggcatggacgagctgtacaagtaataagcttctcgactagggataacagggtaattgtttgaatgaggcttcagtactt |
| | tacagaatcgttgcctgcacatcttggaaacacttgctgggattacttcttcaggttaacccaacagaaggctcgaga |
| | aggtatattgctgttgacagtgagcgcgtacagtatttatcgagataatagtgaagccacagatgtattatctcgataa |
| | atactgtacatgcctactgcctcgcaattgaaggggctactttaggagcaattatcttgtttactaaaactgaataccttg |
| | ctatctctttgatacatttttacaaagctgaattaaaatggtataaattaaatcacttttataaattaaatcacttttttacgcg |
| | tggatccaatcaacctctggattacaaaatttgtgaaagattgactggtattcttaactatgttgctccttttacgctatgtgg |
| | atacgctgctttaatgcctttgtatcatgctattgcttcccgtatggctttcattttctcctccttgtataaatcctggttgctg |
| | tctctttatgaggagttgtggcccgttgtcaggcaacgtggcgtggtgtgcactgtgtttgctgacgcaacccccactggttg |
| | gggcattgccaccacctgtcagctcctttccgggactttcgctttccccctccctattgccacggcggaactcatcgccg |
| | cctgccttgcccgctgctggacaggggctcggctgttgggcactgacaattccgtggtgttgtcggggaaatcatcgtc |
| | ctttccttggctgctcgcctgtgttgccacctggattctgcgcgggacgtccttctgctacgtcccttcggccctcaatcca |
| | gcggaccttccttcccgcggcctgctgccggctctgcggcctcttccgcgtcttcgagatctgcctcgactgtgccttcta |
| | gttgccagccatctgttgtttgcccctcccccgtgccttccttgaccctggaaggtgccactcccactgtcctttcctaata |
| | aaatgaggaaattgcatcgcattgtctgagtaggtgtcattctattctggggggtggggtggggcaggacagcaagg |
| | gggaggattgggaagacaatagcaggcatgctggggagagctcttaagggcgaattcccgataaggatcttccta |
| | gagcatggctacgtagataagtagcatggcgggttaatcattaactacaaggaacccctagtgatggagttggcca |
| | ctccctctctgcgcgctcgctcgctcactgaggccgggcgaccaaaggtcgcccgacgcccgggctttgcccgggc |
| | ggcctcagtgagcgagcgagcgcgcagccttaattaacctaattcactggccgtcgttttacaacgtcgtgactggg |
| | aaaaccctggcgttacccaacttaatcgccttgcagcacatccccctttcgccagctggcgtaatagcgaagaggcc |
| | cgcaccgatcgcccttcccaacagttgcgcagcctgaatggcgaatgggacgcgccctgtagcggcgcattaagc |
| | gcggcgggtgtggtggttacgcgcagcgtgaccgctacacttgccagcgccctagcgcccgctcctttcgctttcttcc |
| | cttcctttctcgccacgttcgccggctttccccgtcaagctctaaatcgggggctccctttagggttccgatttagtgcttta |
| | cggcacctcgaccccaaaaaacttgattagggtgatggttcacgtagtgggccatcgccctgatagacggtttttcgc |
| | cctttgacgttggagtccacgttctttaatagtggactcttgttccaaactggaacaacactcaaccctatctcggtctatt |
| | cttttgatttataagggattttgccgatttcggcctattggttaaaaaatgagctgatttaacaaaaatttaacgcgaatttt |
| | aacaaaatattaacgcttacaatttaggtggcacttttcggggaaatgtgcgcggaacccctatttgtttatttttctaaat |
| | acattcaaatatgtatccgctcatgagacaataaccctgataaatgcttcaataatattgaaaaaggaagagtatgag |
| | ccatattcaacgggaaacgtcgaggccgcgattaaattccaacatggatgctgatttatatgggtataaatgggctcg |
| | cgataatgtcgggcaatcaggtgcgacaatctatcgcttgtatgggaagcccgatgcgccagagttgtttctgaaaca |
| | tggcaaaggtagcgttgccaatgatgttacagatgagatggtcagactaaactggctgacggaatttatgcctcttcc |
| | gaccatcaagcattttatccgtactcctgatgatgcatggttactcaccactgcgatccccggaaaaacagcattcca |
| | ggtattagaagaatatcctgattcaggtgaaaatattgttgatgcgctggcagtgttcctgcgccggttgcattcgattcc |
| | tgtttgtaattgtccttttaacagcgatcgcgtatttcgtcttgctcaggcgcaatcacgaatgaataacggtttggttgatg |
| | cgagtgattttgatgacgagcgtaatggctggcctgttgaacaagtctggaaagaaatgcataaacttttgccattctc |
| | accggattcagtcgtcactcatggtgatttctcacttgataaccttatttttgacgaggggaaattaataggttgtattgatg |
| | ttggacgagtcggaatcgcagaccgataccaggatcttgccatcctatggaactgcctcggtgagttttctccttcatta |
| | cagaaacggctttttcaaaaatatggtattgataatcctgatatgaataaattgcagtttcatttgatgctcgatgagttttt |
| | ctaactgtcagaccaagtttactcatatatactttagattgatttaaaacttcatttttaatttaaaaggatctaggtgaaga |
| | tcctttttgataatctcatgaccaaaatcccttaacgtgagttttcgttccactgagcgtcagaccccgtagaaaagatc |
| | aaaggatcttcttgagatcctttttttctgcgcgtaatctgctgcttgcaaacaaaaaaaccaccgctaccagcggtggt |
| | ttgtttgccggatcaagagctaccaactctttttccgaaggtaactggcttcagcagagcgcagataccaaatactgtt |
| | cttctagtgtagccgtagttaggccaccacttcaagaactctgtagcaccgcctacatacctcgctctgctaatcctgtt |
| | accagtggctgctgccagtggcgataagtcgtgtcttaccgggttggactcaagacgatagttaccggataaggcgc |
| | agcggtcgggctgaacggggggttcgtgcacacagcccagcttggagcgaacgacctacaccgaactgagata |
| | cctacagcgtgagctatgagaaagcgccacgcttcccgaagggagaaaggcggacaggtatccggtaagcggc |
| | agggtcggaacaggagagcgcacgagggagcttccagggggaaacgcctggtatctttatagtcctgtcgggtttc |
| | gccacctctgacttgagcgtcgatttttgtgatgctcgtcaggggggcggagcctatggaaaaacgccagcaacgc |
| | ggcctttttacggttcctggccttttgctggccttttgctcacatgttctttcctgcgttatcccctgattctgtggataaccgt |
| | attaccgcctttgagtgagctgataccgctcgccgcagccgaacgaccgagcgcagcgagtcagtgagcgaggaag |
| | cggaagagcgcccaatacgcaaaccgcctctccccgcgcgttggccgattcattaatgcagctggcacgacaggt |
| | ttcccgactggaaagcgggcagtgagcgcaacgcaattaatgtgagttagctcactcattaggcaccccaggcttta |
| | cactttatgcttccggctcgtatgttgtgtggaattgtgagcggataacaatttcacacaggaaacagctatgaccatg |
| | attacgccagatttaattaagg |
| 33 | ccttaattaggctgcgcgctcgctcgctcactgaggccgcccgggcaaagcccgggcgtcgggcgacctttggtcg |
| (P901) | cccggcctcagtgagcgagcgagcgcgcagagagggagtggccaactccatcactaggggttccttgtagttaat |
| 5′-mirE | gattaacccgccatgctacttatctacgtagccatgctctaggaagatcggaattcgcccttaagctagcggcgcgcc |
| sequence at | accggtgcgatcgccgttacataacttacggtaaatggcccgcctggctgaccgcccaacgacccccgcccattga |
| positions | cgtcaataatgacgtatgttcccatagtaacgccaatagggactttccattgacgtcaatgggtggagtatttacggta |
| 2109- | aactgcccacttggcagtacatcaagtgtatcatatgccaagtacgccccctattgacgtcaatgacggtaaatggcc |
| 2233 | cgcctggcattatgcccagtacatgaccttatgggactttcctacttggcagtacatctacgtattagtcatcgctattacc |
| shRNA_Sox2_4 | atggtgatgcggttttggcagtacatcaatgggcgtggatagcggtttgactcacggggatttccaagtctccacccca |
| sequence at | ttgacgtcaatgggagtttgttttggcaccaaaatcaacgggactttccaaaatgtcgtaacaactccgccccattgac |
| positions | gcaaatgggcggtaggcgtgtacggtgggaggtctatataagcagagctggtttagtgaaccgtcagatcctgcag |
| 2234- | aagttggtcgtgaggcactgggcaggtaagtatcaaggttacaagacaggtttaaggagaccaatagaaactggg |
| 2296 | cttgtcgagacagagaagactcttgcgtttctgataggcacctattggtcttactgacatccactttgcctttctctccaca |
| 3′-mirE | ggtgtccaggcggccgcgccaccatgccagagccagcgaagtctgctcccgccccgaaaaagggctccaagaa |
| sequence at | ggcggtgactaaggcgcagaagaaaggcggcaagaagcgcaagcgcagccgcaaggagagctattccatcta |
| positions | tgtgtacaaggttctgaagcaggtccaccctgacaccggcatttcgtccaaggccatgggcatcatgaattcgtttgtg |
| 2297- | aacgacattttcgagcgcatcgcaggtgaggcttcccgcctggcgcattacaacaagcgctcgaccatcacctcca |
| 2408 | gggagatccagacggccgtgcgcctgctgctgcctggggagttggccaagcacgccgtgtccgagggtactaag |
| | gccatcaccaagtacaccagcgctaaggatccaccggtcgccaccatggtgagcaagggcgaggagctgttcac |
| | cggggtggtgcccatcctggtcgagctggacggcgacgtaaacggccacaagttcagcgtgtccggcgagggcg |
| | agggcgatgccacctacggcaagctgaccctgaagttcatctgcaccaccggcaagctgcccgtgccctggccca |
| | ccctcgtgaccaccctgacctacggcgtgcagtgcttcagccgctaccccgaccacatgaagcagcacgacttcttc |
| | aagtccgccatgcccgaaggctacgtccaggagcgcaccatcttcttcaaggacgacggcaactacaagacccg |
| | cgccgaggtgaagttcgagggcgacaccctggtgaaccgcatcgagctgaagggcatcgacttcaaggaggac |
| | ggcaacatcctggggcacaagctggagtacaactacaacagccacaacgtctatatcatggccgacaagcaga |
| | agaacggcatcaaggtgaacttcaagatccgccacaacatcgaggacggcagcgtgcagctcgccgaccacta |
| | ccagcagaacacccccatcggcgacggccccgtgctgctgcccgacaaccactacctgagcacccagtccgcc |
| | ctgagcaaagaccccaacgagaagcgcgatcacatggtcctgctggagttcgtgaccgccgccgggatcactctc |
| | ggcatggacgagctgtacaagtaataagcttctcgactagggataacagggtaattgtttgaatgaggcttcagtactt |
| | tacagaatcgttgcctgcacatcttggaaacacttgctgggattacttcgacttcttaacccaacagaaggctcgaga |
| | aggtatattgctgttgacagtgagcgCgtacagtatttatcgagataatagtgaagccacagatgtattatctcgataa |
| | atactgtacAtgcctactgcctcggacttcaaggggctagaattcgagcaattatcttgtttactaaaactgaatacctt |
| | gctatctctttgatacatttttacaaagctgaattaaaatggtataaattaaatcacttttttcaattgacgcgtaattctacc |
| | ggatccaatcaacctctggattacaaaatttgtgaaagattgactggtattcttaactatgttgctccttttacgctatgtgg |
| | atacgctgctttaatgcctttgtatcatgctattgcttcccgtatggctttcattttctcctccttgtataaatcctggttgctg |
| | tctctttatgaggagttgtggcccgttgtcaggcaacgtggcgtggtgtgcactgtgtttgctgacgcaacccccactggttg |
| | gggcattgccaccacctgtcagctcctttccgggactttcgctttccccctccctattgccacggcggaactcatcgccg |
| | cctgccttgcccgctgctggacaggggctcggctgttgggcactgacaattccgtggtgttgtcggggaaatcatcgtc |
| | ctttccttggctgctcgcctgtgttgccacctggattctgcgcgggacgtccttctgctacgtcccttcggccctcaatcca |
| | gcggaccttccttcccgcggcctgctgccggctctgcggcctcttccgcgtcttcgagatctgcctcgactgtgccttcta |
| | gttgccagccatctgttgtttgcccctcccccgtgccttccttgaccctggaaggtgccactcccactgtcctttcctaata |
| | aaatgaggaaattgcatcgcattgtctgagtaggtgtcattctattctggggggtggggtggggcaggacagcaagg |
| | gggaggattgggaagacaatagcaggcatgctggggagagctcttaagggcgaattcccgataaggatcttccta |
| | gagcatggctacgtagataagtagcatggcgggttaatcattaactacaaggaacccctagtgatggagttggcca |
| | ctccctctctgcgcgctcgctcgctcactgaggccgggcgaccaaaggtcgcccgacgcccgggctttgcccgggc |
| | ggcctcagtgagcgagcgagcgcgcagccttaattaacctaattcactggccgtcgttttacaacgtcgtgactggg |
| | aaaaccctggcgttacccaacttaatcgccttgcagcacatccccctttcgccagctggcgtaatagcgaagaggcc |
| | cgcaccgatcgcccttcccaacagttgcgcagcctgaatggcgaatgggacgcgccctgtagcggcgcattaagc |
| | gcggcgggtgtggtggttacgcgcagcgtgaccgctacacttgccagcgccctagcgcccgctcctttcgctttcttcc |
| | cttcctttctcgccacgttcgccggctttccccgtcaagctctaaatcgggggctccctttagggttccgatttagtgcttta |
| | cggcacctcgaccccaaaaaacttgattagggtgatggttcacgtagtgggccatcgccctgatagacggtttttcgc |
| | cctttgacgttggagtccacgttctttaatagtggactcttgttccaaactggaacaacactcaaccctatctcggtctatt |
| | cttttgatttataagggattttgccgatttcggcctattggttaaaaaatgagctgatttaacaaaaatttaacgcgaatttt |
| | aacaaaatattaacgcttacaatttaggtggcacttttcggggaaatgtgcgcggaacccctatttgtttatttttctaaat |
| | acattcaaatatgtatccgctcatgagacaataaccctgataaatgcttcaataatattgaaaaaggaagagtatgag |
| | ccatattcaacgggaaacgtcgaggccgcgattaaattccaacatggatgctgatttatatgggtataaatgggctcg |
| | cgataatgtcgggcaatcaggtgcgacaatctatcgcttgtatgggaagcccgatgcgccagagttgtttctgaaaca |
| | tggcaaaggtagcgttgccaatgatgttacagatgagatggtcagactaaactggctgacggaatttatgcctcttcc |
| | gaccatcaagcattttatccgtactcctgatgatgcatggttactcaccactgcgatccccggaaaaacagcattcca |
| | ggtattagaagaatatcctgattcaggtgaaaatattgttgatgcgctggcagtgttcctgcgccggttgcattcgattcc |
| | tgtttgtaattgtccttttaacagcgatcgcgtatttcgtcttgctcaggcgcaatcacgaatgaataacggtttggttgatg |
| | cgagtgattttgatgacgagcgtaatggctggcctgttgaacaagtctggaaagaaatgcataaacttttgccattctc |
| | accggattcagtcgtcactcatggtgatttctcacttgataaccttatttttgacgaggggaaattaataggttgtattgatg |
| | ttggacgagtcggaatcgcagaccgataccaggatcttgccatcctatggaactgcctcggtgagttttctccttcatta |
| | cagaaacggctttttcaaaaatatggtattgataatcctgatatgaataaattgcagtttcatttgatgctcgatgagttttt |
| | ctaactgtcagaccaagtttactcatatatactttagattgatttaaaacttcatttttaatttaaaaggatctaggtgaaga |
| | tcctttttgataatctcatgaccaaaatcccttaacgtgagttttcgttccactgagcgtcagaccccgtagaaaagatc |
| | aaaggatcttcttgagatcctttttttctgcgcgtaatctgctgcttgcaaacaaaaaaaccaccgctaccagcggtggt |
| | ttgtttgccggatcaagagctaccaactctttttccgaaggtaactggcttcagcagagcgcagataccaaatactgtt |
| | cttctagtgtagccgtagttaggccaccacttcaagaactctgtagcaccgcctacatacctcgctctgctaatcctgtt |
| | accagtggctgctgccagtggcgataagtcgtgtcttaccgggttggactcaagacgatagttaccggataaggcgc |
| | agcggtcgggctgaacggggggttcgtgcacacagcccagcttggagcgaacgacctacaccgaactgagata |
| | cctacagcgtgagctatgagaaagcgccacgcttcccgaagggagaaaggcggacaggtatccggtaagcggc |
| | agggtcggaacaggagagcgcacgagggagcttccagggggaaacgcctggtatctttatagtcctgtcgggtttc |
| | gccacctctgacttgagcgtcgatttttgtgatgctcgtcaggggggcggagcctatggaaaaacgccagcaacgc |
| | ggcctttttacggttcctggccttttgctggccttttgctcacatgttctttcctgcgttatcccctgattctgtggataaccgt |
| | attaccgcctttgagtgagctgataccgctcgccgcagccgaacgaccgagcgcagcgagtcagtgagcgaggaag |
| | cggaagagcgcccaatacgcaaaccgcctctccccgcgcgttggccgattcattaatgcagctggcacgacaggt |
| | ttcccgactggaaagcgggcagtgagcgcaacgcaattaatgtgagttagctcactcattaggcaccccaggcttta |
| | cactttatgcttccggctcgtatgttgtgtggaattgtgagcggataacaatttcacacaggaaacagctatgaccatg |
| | attacgccagatttaattaagg |

TABLE 6
Exemplary siRNA sequences

| SEQ ID NO: | Sequence |
|---|---|
| 35 | CCAGUAAUAUUUAGAGCUAUU |
| Sox2 siRNA A-058489-13 | |
| sense strand | |
| 36 | UAGCUCUAAAUAUUACUGGUU |
| Sox2 siRNA A-058489-13 | |
| antisense strand | |
| 37 | CGCUCAUGAAGAAGGAUAAUU |
| Sox2siRNAA-058489-14 | |
| sense strand | |
| 38 | UUAUCCUUCUUCAUGAGCGUU |
| Sox2siRNAA-058489-14 | |
| antisense strand | |
| 39 | UUGUGAUAUUUUAAGGUUUUU |
| Sox2siRNAA-058489-15 | |
| sense strand | |
| 40 | AAACCUUAAAAUAUCACAAUU |
| Sox2siRNAA-058489-15 | |
| antisense strand | |
| 41 | CUUAUGGUUUGUAAUAUUUUU |
| Sox2siRNAA-058489-16 | |
| sense strand | |
| 42 | AAAUAUUACAAACCAUAAGUU |
| Sox2siRNAA-058489-16 | |
| antisense strand | |

In some embodiments, the siRNA or shRNA targeting Sox2 has a nucleobase sequence containing a portion of at least 8 contiguous nucleobases (e.g., 8, 9, 10, 11, 12, 13, 14, 15, 16, 17, 18, 19, 20, or more nucleobases) having at least 70% complementarity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity) to an equal length portion of a target region of an mRNA transcript of a human (e.g., SEQ ID NO: 1) or murine (SEQ ID NO: 3) SOX2 gene. In some embodiments the target region is at least 8 to 21 (e.g., 8 to 21, 9 to 21, 10 to 21, 11 to 21, 12 to 21, 13 to 21, 14 to 21, 15 to 21, 16 to 21, 17 to 21, 18 to 21, 19 to 21, 20 to 21, or all 21) contiguous nucleobases of any one or more of SEQ ID NOs: 5-23. In some embodiments the target region is at least 8 to 19 (e.g., 8 to 19, 9 to 19, 10 to 19, 11 to 19, 12 to 19, 13 to 19, 14 to 19, 15 to 19, 16 to 19, 17 to 19, 18 to 19, or all 19) contiguous nucleobases of any one of SEQ ID NOs: 25-27. In some embodiments the target region is at least 8 to 22 (e.g., 8 to 22, 9 to 22, 10 to 22, 11 to 22, 12 to 22, 13 to 22, 14 to 22, 15 to 22, 16 to 22, 17 to 22, 18 to 22, 19 to 22, 20 to 22, 21 to 22, or all 22) contiguous nucleobases of SEQ ID NOs: 28 or 29.

In some embodiments, the siRNA or shRNA targets SEQ ID NO: 11, SEQ ID NO: 25, SEQ ID NO: 26, or SEQ ID NO: 27, SEQ ID NO: 28, or SEQ ID NO: 29.

In some embodiments, the shRNA has at least 70% complementarity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity) to the entire length of SEQ ID NO: 11, SEQ ID NO: 25, SEQ ID NO: 26, SEQ ID NO: 27, SEQ ID NO: 28, or SEQ ID NO: 29. In some embodiments, the shRNA has 100% complementarity to the entire length of SEQ ID NO: 11, SEQ ID NO: 25, SEQ ID NO: 26, SEQ ID NO: 27, SEQ ID NO: 28, or SEQ ID NO: 29.

In some embodiments, the shRNA includes the sequence of nucleotides 2234-2296 of SEQ ID NO: 30 or nucleotides 2234-2296 of SEQ ID NO: 32. In some embodiments, the shRNA has the sequence of nucleotides 2234-2296 of SEQ ID NO: 30 or nucleotides 2234-2296 of SEQ ID NO: 32. In some embodiments, the shRNA is embedded into the backbone of an miRNA. In some embodiments, the miRNA backbone and the shRNA include the sequence of nucleotides 2109-2426 of SEQ ID NO: 30, nucleotides 2109-2408 of SEQ ID NO: 31, nucleotides 2109-2426 of SEQ ID NO: 32, or nucleotides 2109-2408 of SEQ ID NO: 33. In some embodiments, the miRNA backbone and the shRNA have the sequence of nucleotides 2109-2426 of SEQ ID NO: 30, nucleotides 2109-2408 of SEQ ID NO: 31, nucleotides 2109-2426 of SEQ ID NO: 32, or nucleotides 2109-2408 of SEQ ID NO: 33.

In some embodiments, the siRNA is a pair of nucleotide sequences (sense and anti-sense strands) selected from SEQ ID NO: 35 and SEQ ID NO: 36; SEQ ID NO: 37 and SEQ ID NO: 38; SEQ ID NO: 39 and SEQ ID NO: 40; and SEQ ID NO: 41 and SEQ ID NO: 42.

An miRNA is a non-coding RNA molecule that typically has a length of about 22 nucleotides. miRNAs bind to target sites on messenger RNA (mRNA) molecules and silence the mRNA, e.g., by causing cleavage of the mRNA, destabilization of the mRNA, or inhibition of translation of the mRNA.

siRNA, shRNA, and miRNA molecules for use in the methods and compositions described herein can target the mRNA sequence of Sox2 (e.g., human Sox2 mRNA, which has the sequence of SEQ ID NO: 1, or murine Sox2 mRNA, which has the sequence of SEQ ID NO: 3). An miRNA that targets a Sox2 promoter can also be used to silence Sox2. Exemplary miRNA molecules that inhibit Sox2 include human miRNAs miR-145, miR-126, miR-200c, miR-429, miR-200b, miR-140, miR-9, miR-21, miR-590, miR-182, and miR-638, and murine miRNAs miR-134, miR-200c, miR-429, miR-200b, miR-34a, and miR-9. siRNA molecules may be delivered without a delivery vehicle, or they may be encapsulated in a nanoparticle (e.g., a lipid nanoparticle) for administration. siRNA, shRNA, and miRNA molecules may be delivered using a plasmid, such as a viral vector (e.g., an AAV vector), and they may be expressed using a cell type-specific promoter (e.g., a hair cell promoter, Type II hair cell promoter, or a supporting cell promoter) or using a ubiquitous promoter (e.g., a ubiquitous pol II or pol III promoter).

An inhibitory RNA molecule can be modified, e.g., to contain modified nucleotides, e.g., 2′-fluoro, 2′-o-methyl, 2′-deoxy, unlocked nucleic acid, 2′-hydroxy, phosphorothioate, 2′-thiouridine, 4′-thiouridine, 2′-deoxyuridine. Without wishing to be bound by theory, it is believed that certain modifications can increase nuclease resistance and/or serum stability or decrease immunogenicity.

In some embodiments, the inhibitory RNA molecule decreases the level and/or activity or function of Sox2. In some embodiments, the inhibitory RNA molecule inhibits expression of Sox2. In other embodiments, the inhibitor RNA molecule increases degradation of Sox2 and/or decreases the stability (i.e., half-life) of Sox2. The inhibitory RNA molecule can be chemically synthesized or transcribed in vitro.

The making and use of inhibitory therapeutic agents based on non-coding RNA such as ribozymes, RNAse P, siRNAs, and miRNAs are also known in the art, for example, as described in Sioud, RNA Therapeutics: Function, Design, and Delivery (Methods in Molecular Biology). Humana Press 2010.

Gene Editing

In some embodiments, the Sox2 inhibitor is a component of a gene editing system. For example, the Sox2 inhibitor introduces an alteration (e.g., insertion, deletion (e.g., knockout), translocation, inversion, single point mutation, or other mutation) in Sox2. Exemplary gene editing systems include the zinc finger nucleases (ZFNs), transcription activator-like effector-based nucleases (TALEN), and the clustered regulatory interspaced short palindromic repeat (CRISPR) system. ZFNs, TALENs, and CRISPR-based methods are described, e.g., in Gaj et al. Trends Biotechnol. 31.7(2013):397-405.

A CRISPR system refers to a system derived from CRISPR and Cas (a CRISPR-associated protein) or other nuclease that can be used to silence or mutate a gene described herein. The CRISPR system is a naturally occurring system found in bacterial and archaeal genomes. The CRISPR locus is made up of alternating repeat and spacer sequences. In naturally-occurring CRISPR systems, the spacers are typically sequences that are foreign to the bacterium (e.g., plasmid or phage sequences). The CRISPR system has been modified for use in gene editing (e.g., changing, silencing, and/or enhancing certain genes) in eukaryotes. See, e.g., Wiedenheft et al., Nature 482: 331, 2012. For example, such modification of the system includes introducing into a eukaryotic cell a plasmid containing a specifically-designed CRISPR and one or more appropriate Cas proteins. The CRISPR locus is transcribed into RNA and processed by Cas proteins into small RNAs that contain a repeat sequence flanked by a spacer. The RNAs serve as guides to direct Cas proteins to silence specific DNA/RNA sequences, depending on the spacer sequence. See, e.g., Horvath et al., Science 327: 167, 2010; Makarova et al., Biology Direct 1:7, 2006; Pennisi, Science 341: 833, 2013. In some examples, the CRISPR system includes the Cas9 protein, a nuclease that cuts on both strands of the DNA. See, e.g., Id. In some embodiments, a CRISPR-Cpf1 system can be used, which is an RNA-guided, class II CRISPR/Cas system that is analogous to CRISPR-Cas9 and has lower off-target incidence (see Moon et al., Nature Communications 9, 3651 (2018)). In embodiments in which a CRISPR-Cpf1 system is used, the guide RNA can be embedded in the untranslated region of the transgene used to deliver the gene editing system.

In some embodiments, in a CRISPR system for use described herein, e.g., in accordance with one or more methods described herein, the spacers of the CRISPR are derived from a target gene sequence, e.g., from a Sox2 sequence.

In some embodiments, the Sox2 inhibitor includes a guide RNA (gRNA) for use in a CRISPR system for gene editing. In some embodiments, the Sox2 inhibitor is a ZFN, or an mRNA encoding a ZFN, that targets (e.g., cleaves) a nucleic acid sequence (e.g., DNA sequence) of Sox2. In some embodiments, the Sox2 inhibitor includes a TALEN, or an mRNA encoding a TALEN, that targets (e.g., cleaves) a nucleic acid sequence (e.g., DNA sequence) of Sox2.

For example, the gRNA can be used in a CRISPR system to engineer an alteration in a gene (e.g., Sox2). In other examples, the ZFN and/or TALEN can be used to engineer an alteration in a gene (e.g., Sox2). Exemplary alterations include insertions, deletions (e.g., knockouts), translocations, inversions, single point mutations, or other mutations. The alteration can be introduced in the gene in a cell, e.g., in vitro, ex vivo, or in vivo. In some embodiments, the alteration decreases the level and/or activity of (e.g., knocks down or knocks out) Sox2, e.g., the alteration is a negative regulator of function.

In certain embodiments, the CRISPR system is used to edit (e.g., to add or delete a base pair) a target gene, e.g., Sox2. In other embodiments, the CRISPR system is used to introduce a premature stop codon, e.g., thereby decreasing the expression of a target gene. In yet other embodiments, the CRISPR system is used to turn off a target gene in a reversible manner, e.g., similarly to RNA interference. In embodiments, the CRISPR system is used to direct Cas to a promoter of a target gene, e.g., Sox2, thereby blocking an RNA polymerase sterically.

In some embodiments, a CRISPR system can be generated to edit Sox2 using technology described in, e.g., U.S. Publication No. 20140068797; Cong, Science 339: 819, 2013; Tsai, Nature Biotechnol., 32:569, 2014; and U.S. Pat. Nos. 8,871,445; 8,865,406; 8,795,965; 8,771,945; and 8,697,359.

In some embodiments, the CRISPR interference (CRISPRi) technique can be used for transcriptional repression of specific genes, e.g., the gene encoding Sox2. In CRISPRi, an engineered Cas9 protein (e.g., nuclease-null dCas9, or a dCas9 fusion protein, e.g., dCas9-KRAB or dCas9-SID4X fusion) can pair with a sequence specific guide RNA (sgRNA). The Cas9-gRNA complex can block RNA polymerase, thereby interfering with transcription elongation. The complex can also block transcription initiation by interfering with transcription factor binding. The CRISPRi method is specific with minimal off-target effects and is multiplexable, e.g., can simultaneously repress more than one gene (e.g., using multiple gRNAs). Also, the CRISPRi method permits reversible gene repression.

The components of the gene editing system can be delivered using a plasmid, e.g., a viral vector, such as an AAV vector. A hair cell, Type II hair cell, or supporting cell promoter can be used to direct expression of the gene editing system in regenerated or regenerating hair cells, in Type II hair cells, or in vestibular supporting cells to knockdown or knockout Sox2 in these cell types and convert them to Type I hair cells. In some embodiments, the components of the gene editing system or a plasmid (e.g., AAV vector) encoding the components of the gene editing system are delivered using a nanoparticle (e.g., a lipid nanoparticle).

Dominant Negative Sox2 Protein

In some embodiments, the Sox2 inhibitor is a dominant negative Sox2 protein. The dominant negative Sox2 protein can be delivered to regenerated or regenerating hair cells, to Type II hair cells, or to vestibular supporting cells using a plasmid, e.g., a viral vector, such as an AAV vector, containing a polynucleotide encoding the dominant negative Sox2 protein. The polynucleotide encoding the dominant negative Sox2 protein can be operably linked to a promoter that induces expression in a cell type of interest, e.g., in a hair cell, Type II hair cell, or vestibular supporting cell. The dominant negative Sox2 protein may be produced by mutating the two nuclear localization signals in the high mobility group domain of Sox2 (as described in Li et al., J Biol Chem 282:19481-92 (2007)), by generating a Sox2 polynucleotide that lacks all or most of the high mobility group domain (as described in Kishi et al., Development 127:791-800 (2000)), by generating a Sox2 polynucleotide in which the high mobility group domain is fused with the engrailed repressor domain (as described in Kishi et al., Development 127:791-800 (2000)), or by generating a Sox2 polynucleotide that only encodes the Sox2 DNA binding domain (e.g., a C-terminally truncated version of Sox2 that can compete with wild-type Sox2 by binding to Sox2 recognition sites on DNA but that lacks a transactivation domain, e.g., as described in Pan and Schultz, Biology of Reproduction 85:409-416 (2011), Hutz et al., Carcinogenesis 35:942-950 (2013), and Gaete et al., Neural Development 7:13 (2012)). In some embodiments, the dominant negative Sox2 protein is encoded by the sequence:

(SEQ ID NO: 24)
ATGTATAACATGATGGAGACGGAGCTGAAGCCGCCGGGCCCGCAGCAAGCT
TCGGGGGGCGGCGGCGGAGGAGGCAACGCCACGGCGGCGGCGACCGGCGGC
AACCAGAAGAACAGCCCGGACCGCGTCACGGGGCCCATGAACGCCTTCATG
GTATGGTCCCGGGGGCAGCTGGGTAAGATGGCCCAGGAGAACCCCAAGATG
CACAACTCGGAGATCAGCAAGCGCCTGGGCGCGGAGTGGAAACTTTTGTCC
GAGACCGAGAAGCGGCCGTTCATCGACGAGGCCAAGCGGCTGCGCGCTCTG
CACATGAAGGAGCACCCGGATTATAAATACCGGCCGCTGGGGAAAACCAAG
ACGCTCATGAAGAAGGATAAGTACACGCTTCCCGGAGGCTTGCTGGCCCCC
GGCGGGAACAGCATGGCGAGCGGGGTTGGGGTGGGCGCCGGCCTGGGTGCG
GGCGTGAACCAGCGCATGGACAGCTACGCGCACATGAACGGCTGGAGCAAC
GGCAGCTACAGCATGATGCAGGAGCAGCTGGGCTACCCGCAGCACCCGGGC
CTCAACGCTCACGGCGCGGCACAGATGCAACCGATGCACCGCTACGACGTC
AGCGCCCTGCAGTACAACTCCATGACCAGCTCGCAGACCTACATGAACGGC
TCGCCCACCTACAGCATGTCCTACTCGCAGCAGGGCACCCCCGGTATGGCG
CTGGGCTCCATGGGCTCTGTGGTCAAGTCCGAGGCCAGCTCCAGCCCCCCC
GTGGTTACCTCTTCCTCCCACTCCAGGGCGCCCTGCCAGGCCGGGGACCTC
CGGGACATGATCAGCATGTACCTCCCCGGCGCCGAGGTGCCGGAGCCCGCT
GCGCCCAGTAGACTGCACATGGCCCAGCACTACCAGAGCGGCCCGGTGCCC
GGCACGGCCATTAACGGCACACTGCCCCTGTCGCAC;

or the sequence:

(SEQ ID NO: 34)
ATGTATAACATGATGGAGACGGAGCTGAAGCCGCCGGGCCCGCAGCAAGCT
TCGGGGGGCGGCGGCGGAGGAGGCAACGCCACGGCGGCGGCGACCGGCGGC
AACCAGAAGAACAGCCCGGACCGCGTCACGGGGCCCATGAACGCCTTCATG
GTATGGTCCCGGGGGCAGCTGGGTAAGATGGCCCAGGAGAACCCCAAGATG
CACAACTCGGAGATCAGCAAGCGCCTGGGCGCGGAGTGGAAACTTTTGTCC
GAGACCGAGAAGCGGCCGTTCATCGACGAGGCCAAGCGGCTGCGCGCTCTG
CACATGAAGGAGCACCCGGATTATAAATACCGGCCGCTGGGGAAAACCAAG
ACGCTCATGAAGAAGGATAAGTACACGCTTCCCGGAGGCTTGCTGGCCCCC
GGCGGGAACAGCATGGCGAGCGGGGTTGGGGTGGGCGCCGGCCTGGGTGCG
GGCGTGAACCAGCGCATGGACAGCTACGCGCACATGAACGGCTGGAGCAAC
GGCAGCTACAGCATGATGCAGGAGCAGCTGGGCTACCCGCAGCACCCGGGC
CTCAACGCTCACGGCGCGGCACAGATGCAACCGATGCACCGCTACGACGTC
AGCGCCCTGCAGTACAACTCCATGACCAGCTCGCAGACCTACATGAACGGC
TCGCCCACCTACAGCATGTCCTACTCGCAGCAGGGCACCCCCGGTATGGCG
CTGGGCTCCATGGGCTCTGTGGTCAAGTCCGAGGCCAGCTCCAGCCCCCCC
GTGGTTACCTCTTCCTCCCACTCCAGGGCGCCCTGCCAGGCCGGGGACCTC
CGGGACATGATCAGCATGTACCTCCCCGGCGCCGAGGTGCCGGAGCCCGCT
GCGCCCAGTAGACTGCACATGGCCCAGCACTACCAGAGCGGCCCGGTGCCC
GGCACGGCCATTAACGGCACACTGCCCCTGTCGCACATG.

### Hair Cell Regeneration

In some embodiments, the methods described herein are performed by converting Type II vestibular hair cells directly into Type I vestibular hair cells. In such embodiments, the Sox2 inhibitors described herein may be targeted to Type II hair cells using Type II hair cell promoters. The methods described herein may also be performed by targeting Sox2 inhibitors to hair cells produced by regeneration (regenerated hair cells) or hair cells currently undergoing regeneration (regenerating hair cells). Hair cell promoters can be used to target regenerated or regenerating hair cells. In other embodiments, Sox2 inhibitors may be targeted to vestibular supporting cells using a supporting cell promoter in advance of regeneration.

In some embodiments, Sox2 inhibition using an inhibitor described herein (e.g., an inhibitory RNA targeting Sox2 mRNA or a Sox2 promoter, a component of a gene editing system targeting Sox2, or a dominant negative Sox2 protein) is performed in conjunction with a method that promotes hair cell regeneration. One method of promoting hair cell regeneration includes overexpression of Atonal BHLH transcription factor 1 (Atoh1) in vestibular supporting cells. Atoh1 can be overexpressed using a vector, e.g., a viral vector, such as an AAV vector, adenoviral vector, or lentiviral vector, to deliver a polynucleotide encoding Atoh1 to the inner ear. Methods of overexpressing Atoh1 in the inner ear to promote regeneration are described in U.S. Pat. Nos. 7,442,688, 6,838,444, and 9,951,351, and in U.S. Patent Application Nos. US20050281786, US20140005257, US20170314027, US20170327557A1, and US20140134136A1, which are incorporated herein by reference. In some embodiments, the polynucleotide encoding Atoh1 is operably linked to a ubiquitous promoter. In some embodiments, the polynucleotide encoding Atoh1 is operably linked to a supporting cell promoter to induce expression in the desired target cell. Atoh1 expression may also be increased using small molecules, as described in U.S. Pat. Nos. 8,188,131 and 10,143,711 and U.S. Patent Application Nos. US20170042842 and US20190203210, which are incorporated herein by reference.

For Atoh1 overexpression, the polynucleotide sequence encoding Atoh1 can be a polynucleotide sequence that encodes wild-type Atoh1, or a variant thereof, such as a polynucleotide sequence that encodes a protein having at least 85% sequence identity (e.g., 85%, 90%, 95%, 96%, 97%, 98%, 99%, or more, sequence identity) to the amino acid sequence of wild-type mammalian (e.g., human or mouse) Atoh1 (e.g., SEQ ID NO: 43 or SEQ ID NO: 45). Exemplary Atoh1 amino acid and polynucleotide sequences are listed in Table 7, below. In some embodiments, the polynucleotide sequence encoding Atoh1 encodes an amino acid sequence that contains one or more conservative amino acid substitutions relative to SEQ ID NO: 43 (e.g., 1, 2, 3, 4, 5, 6, 7, 8, 9, 10, 11, 12, 13, 14, 15, 16, 17, 18, 19, 20 or more conservative amino acid substitutions), provided that the Atoh1 analog encoded retains the therapeutic function of wild-type Atoh1 (e.g., the ability to promote hair cell development). No more than 10% of the amino acids in the Atoh1 protein may be replaced with conservative amino acid substitutions. In some embodiments, the polynucleotide sequence that encodes Atoh1 is any polynucleotide sequence that, by redundancy of the genetic code, encodes SEQ ID NO: 43. The polynucleotide sequence that encodes Atoh1 can be partially or fully codon-optimized for expression (e.g. in human vestibular supporting cells). Atoh1 may be encoded by a polynucleotide having the sequence of SEQ ID NO: 44. The Atoh1 protein may be a human Atoh1 protein or may be a homolog of the human Atoh1 protein from another mammalian species (e.g., mouse, rat, cow, horse, goat, sheep, donkey, cat, dog, rabbit, guinea pig, or other mammal).

TABLE 7
Atoh1 sequences

| SEQ | Description of | |
|---|---|---|
| ID NO: | sequence | Sequence |
| 43 | Human Atoh1 amino | MSRLLHAEEWAEVKELGDHHRQPQPHHLPQPPPPPQPPATLQARE |
| | acid sequence, | HPVYPPELSLLDSTDPRAWLAPTLQGICTARAAQYLLHSPELGASEA |
| | RefSeq accession | AAPRDEVDGRGELVRRSSGGASSSKSPGPVKVREQLCKLKGGVVV |
| | number | DELGCSRQRAPSSKQVNGVQKQRRLAANARERRRMHGLNHAFDQL |
| | NP_005163.1 | RNVIPSFNNDKKLSKYETLQMAQIYINALSELLQTPSGGEQPPPPPAS |
| | | CKSDHHHLRTAASYEGGAGNATAAGAQQASGGSQRPTPPGSCRTR |
| | | FSAPASAGGYSVQLDALHFSTFEDSALTAMMAQKNLSPSLPGSILQP |
| | | VQEENSKTSPRSHRSDGEFSPHSHYSDSDEAS |
| 44 | Human ATOH1 | ATGTCCCGCCTGCTGCATGCAGAAGAGTGGGCTGAAGTGAAGGA |
| | protein coding | GTTGGGAGACCACCATCGCCAGCCCCAGCCGCATCATCTCCCGC |
| | sequence, also | AACCGCCGCCGCCGCCGCAGCCACCTGCAACTTTGCAGGCGAGA |
| | documented under | GAGCATCCCGTCTACCCGCCTGAGCTGTCCCTCCTGGACAGCAC |
| | RefSeq accession | CGACCCACGCGCCTGGCTGGCTCCCACTTTGCAGGGCATCTGCA |
| | number | CGGCACGCGCCGCCCAGTATTTGCTACATTCCCCGGAGCTGGGT |
| | NM_005172.2 | GCCTCAGAGGCCGCTGCGCCCCGGGACGAGGTGGACGGCCGGG |
| | | GGGAGCTGGTAAGGAGGAGCAGCGGCGGTGCCAGCAGCAGCAA |
| | | GAGCCCCGGGCCGGTGAAAGTGCGGGAACAGCTGTGCAAGCTG |
| | | AAAGGCGGGGTGGTGGTAGACGAGCTGGGCTGCAGCCGCCAAC |
| | | GGGCCCCTTCCAGCAAACAGGTGAATGGGGTGCAGAAGCAGAGA |
| | | CGGCTAGCAGCCAACGCCAGGGAGCGGCGCAGGATGCATGGGC |
| | | TGAACCACGCCTTCGACCAGCTGCGCAATGTTATCCCGTCGTTCA |
| | | ACAACGACAAGAAGCTGTCCAAATATGAGACCCTGCAGATGGCCC |
| | | AAATCTACATCAACGCCTTGTCCGAGCTGCTACAAACGCCCAGCG |
| | | GAGGGGAACAGCCACCGCCGCCTCCAGCCTCCTGCAAAAGCGAC |
| | | CACCACCACCTTCGCACCGCGGCCTCCTATGAAGGGGGCGCGGG |
| | | CAACGCGACCGCAGCTGGGGCTCAGCAGGCTTCCGGAGGGAGC |
| | | CAGCGGCCGACCCCGCCCGGGAGTTGCCGGACTCGCTTCTCAGC |
| | | CCCAGCTTCTGCGGGAGGGTACTCGGTGCAGCTGGACGCTCTGC |
| | | ACTTCTCGACTTTCGAGGACAGCGCCCTGACAGCGATGATGGCG |
| | | CAAAAGAATTTGTCTCCTTCTCTCCCCGGGAGCATCTTGCAGCCA |
| | | GTGCAGGAGGAAAACAGCAAAACTTCGCCTCGGTCCCACAGAAG |
| | | CGACGGGGAATTTTCCCCCCATTCCCATTACAGTGACTCGGATGA |
| | | GGCAAGT |
| 45 | Murine Atoh1 amino | MSRLLHAEEWAEVKELGDHHRHPQPHHVPPLTPQPPATLQARDLPV |
| | acid sequence, | YPAELSLLDSTDPRAWLTPTLQGLCTARAAQYLLHSPELGASEAAAP |
| | UniProt P48985 | RDEADSQGELVRRSGCGGLSKSPGPVKVREQLCKLKGGVVVDELG |
| | | CSRQRAPSSKQVNGVQKQRRLAANARERRRMHGLNHAFDQLRNVI |
| | | PSFNNDKKLSKYETLQMAQIYINALSELLQTPNVGEQPPPPTASCKN |
| | | DHHHLRTASSYEGGAGASAVAGAQPAPGGGPRPTPPGPCRTRFSG |
| | | PASSGGYSVQLDALHFPAFEDRALTAMMAQKDLSPSLPGGILQPVQ |
| | | EDNSKTSPRSHRSDGEFSPHSHYSDSDEAS |
| 46 | Murine ATOH1 | ATGTCCCGCCTGCTGCATGCAGAAGAGTGGGCTGAGGTAAAAGA |
| | protein coding | GTTGGGGGACCACCATCGCCATCCCCAGCCGCACCACGTCCCGC |
| | sequence, also | CGCTGACGCCACAGCCACCTGCTACCCTGCAGGCGAGAGACCTT |
| | documented under | CCCGTCTACCCGGCAGAACTGTCCCTCCTGGATAGCACCGACCC |
| | RefSeq accession | ACGCGCCTGGCTGACTCCCACTTTGCAGGGCCTCTGCACGGCAC |
| | number | GCGCCGCCCAGTATCTGCTGCATTCTCCCGAGCTGGGTGCCTCC |
| | NM_007500.5 | GAGGCCGCGGCGCCCCGGGACGAGGCTGACAGCCAGGGTGAGC |
| | | TGGTAAGGAGAAGCGGCTGTGGCGGCCTCAGCAAGAGCCCCGG |
| | | GCCCGTCAAAGTACGGGAACAGCTGTGCAAGCTGAAGGGTGGGG |
| | | TTGTAGTGGACGAGCTTGGCTGCAGCCGCCAGCGAGCCCCTTCC |
| | | AGCAAACAGGTGAATGGGGTACAGAAGCAAAGGAGGCTGGCAGC |
| | | AAACGCAAGGGAACGGCGCAGGATGCACGGGCTGAACCACGCCT |
| | | TCGACCAGCTGCGCAACGTTATCCCGTCCTTCAACAACGACAAGA |
| | | AGCTGTCCAAATATGAGACCCTACAGATGGCCCAGATCTACATCA |
| | | ACGCTCTGTCGGAGTTGCTGCAGACTCCCAATGTCGGAGAGCAA |
| | | CCGCCGCCGCCCACAGCTTCCTGCAAAAATGACCACCATCACCTT |
| | | CGCACCGCCTCCTCCTATGAAGGAGGTGCGGGCGCCTCTGCGGT |
| | | AGCTGGGGCTCAGCCAGCCCCGGGAGGGGGCCCGAGACCTACC |
| | | CCGCCCGGGCCTTGCCGGACTCGCTTCTCAGGCCCAGCTTCCTC |
| | | TGGGGGTTACTCGGTGCAGCTGGACGCTTTGCACTTCCCAGCCTT |
| | | CGAGGACAGGGCCCTAACAGCGATGATGGCACAGAAGGACCTGT |
| | | CGCCTTCGCTGCCCGGGGGCATCCTGCAGCCTGTACAGGAGGAC |
| | | AACAGCAAAACATCTCCCAGATCCCACAGAAGTGACGGAGAGTTT |
| | | TCCCCCCACTCTCATTACAGTGACTCTGATGAGGCCAGT |

Another method of promoting hair cell regeneration includes inhibition of Notch in vestibular supporting cells. Notch can be inhibited using small molecules (e.g., gamma-secretase inhibitors), inhibitory RNA molecules (e.g., siRNA, shRNA, or miRNA) directed to Notch or to the Notch promoter, or anti-Notch antibodies. Methods of using Notch inhibitors to promote hair cell regeneration are described in U.S. Pat. No. 10,450,317, U.S. Patent Application No. US20190010449, and International Application No. WO2019148067, which are incorporated herein by reference. Inhibitory RNA molecules directed to Notch or to the Notch promoter can be delivered using a vector, e.g., a viral vector, such as an AAV vector, adenoviral vector, or lentiviral vector, and operably linked to a ubiquitous promoter or to a supporting cell promoter, which can be used induce expression in the desired target cell.

In some embodiments, the Sox2 inhibitor is administered in combination with an agent that promotes hair cell regeneration (e.g., an agent that increases Atoh1 overexpression and/or a Notch inhibitor). If the Sox2 inhibitor is administered concurrently with or after the agent that promotes hair cell regeneration, the Sox2 inhibitor can be targeted to Type II hair cells and/or to regenerating or regenerated hair cells. In embodiments in which the Sox2 inhibitor and the regeneration agent are concurrently delivered using vectors, e.g., viral vectors, such as AAV vectors, adenoviral vectors, or lentiviral vectors (e.g., in embodiments in which the Sox2 inhibitor is an inhibitory RNA targeting Sox2, a component of a gene editing system targeting Sox2, or a dominant negative Sox2 protein and the regeneration agent is a polynucleotide encoding Atoh1 or an inhibitory RNA targeting Notch), the Sox2 inhibitor and the regeneration agent can be delivered using separate vectors or using a single vector (e.g., a single AAV vector can include both the Sox2 inhibitor and the regeneration agent). If the Sox2 inhibitor and the regeneration agent are delivered using a single vector, they may be expressed using the same promoter (e.g., a ubiquitous promoter or a supporting cell promoter) or using different promoters (e.g., a hair cell or Type II hair cell promoter and a supporting cell promoter). The Sox2 inhibitor and the regeneration agent can also be co-formulated for concurrent administration. In other embodiments, the Sox2 inhibitor is administered prior to administration of an agent that promotes hair cell regeneration. In such embodiments, the Sox2 inhibitor can be targeted to supporting cells (e.g., vestibular supporting cells), which are then induced to regenerate into hair cells using a regeneration agent. The Sox2 inhibitor can also be targeted to regenerated hair cells and/or Type II hair cells even if a regeneration agent has not yet been administered, as spontaneous regeneration may occur in the vestibular system and Type II cells are more likely to be preserved after hair cell damage. The first therapeutic agent (e.g., the regeneration agent or Sox2 inhibitor) may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours, up to 24 hours, or up to 1-7, 1-14, 1-21 or 1-30 days before or after the second therapeutic agent (e.g., the Sox2 inhibitor or the regeneration agent).

### Expression of Exogenous Nucleic Acids in Mammalian Cells

One platform that can be used to achieve therapeutically effective intracellular concentrations of exogenous nucleic acid molecules in mammalian cells is via the stable expression of the nucleic acid molecule (e.g., by integration into the nuclear or mitochondrial genome of a mammalian cell, or by episomal concatemer formation in the nucleus of a mammalian cell). The nucleic acid may be an inhibitory RNA (e.g., an inhibitory RNA targeting Sox2 or Notch) or a polynucleotide that encodes the primary amino acid sequence of a corresponding protein (e.g., a polynucleotide encoding a dominant negative Sox2 protein or Atoh1). In order to introduce exogenous nucleic acid molecules into a mammalian cell, nucleic acid molecules can be incorporated into a vector. Vectors can be introduced into a cell by a variety of methods, including transformation, transfection, transduction, direct uptake, projectile bombardment, and by encapsulation of the vector in a liposome. Examples of suitable methods of transfecting or transforming cells include calcium phosphate precipitation, electroporation, microinjection, infection, lipofection and direct uptake. Such methods are described in more detail, for example, in Green, et al., Molecular Cloning: A Laboratory Manual, Fourth Edition (Cold Spring Harbor University Press, New York 2014); and Ausubel, et al., Current Protocols in Molecular Biology (John Wiley & Sons, New York 2015), the disclosures of each of which are incorporated herein by reference.

Nucleic acid molecules can also be introduced into a mammalian cell by targeting a vector containing a nucleic acid molecule of interest to cell membrane phospholipids. For example, vectors can be targeted to the phospholipids on the extracellular surface of the cell membrane by linking the vector molecule to a VSV-G protein, a viral protein with affinity for all cell membrane phospholipids. Such a construct can be produced using methods well known to those of skill in the field.

Recognition and binding of the nucleic acid molecule by mammalian RNA polymerase is important for gene expression. As such, one may include sequence elements within the polynucleotide that exhibit a high affinity for transcription factors that recruit RNA polymerase and promote the assembly of the transcription complex at the transcription initiation site. Such sequence elements include, e.g., a mammalian promoter, the sequence of which can be recognized and bound by specific transcription initiation factors and ultimately RNA polymerase. Promoter sequences are typically located upstream of the translation start site (e.g., within two kilobases upstream of the translation start site). Examples of mammalian promoters have been described in Smith, et al., Mol. Sys. Biol., 3:73, online publication, the disclosure of which is incorporated herein by reference. The promoter used in the methods and compositions described herein can be a ubiquitous promoter (e.g., to induce or increase expression of the nucleic acid molecule in all cells of the vestibular system) or a cell type-specific promoter (e.g., to induce or increase expression of the nucleic acid molecule in one or more inner ear cell types). Ubiquitous promoters include the CAG promoter, cytomegalovirus (CMV) promoter, the smCBA promoter (described in Haire et al., Invest. Opthalmol. Vis. Sci. 47:3745-3753, 2006), the dihydrofolate reductase (DHFR) promoter, the β-actin promoter, the phosphoglycerol kinase (PGK) promoter, and the EF1α promoter. Alternatively, promoters derived from viral genomes can also be used for the stable expression of polynucleotides in primate (e.g., human) cells. Examples of functional viral promoters that can be used for the expression of polynucleotides in primate (e.g., human) cells include adenovirus late promoter, vaccinia virus 7.5K promoter, tk promoter of HSV, mouse mammary tumor virus (MMTV) promoter, LTR promoter of HIV, promoter of moloney virus, Epstein barr virus (EBV) promoter, and the Rous sarcoma virus (RSV) promoter. A pol II promoter, such as a ubiquitous promoter described above or a promoter described in Table 8, below, can be used to express any Sox2 inhibitor or regeneration agent described herein. A pol III promoter, including ubiquitous pol III promoters U6, H1, and 7SK, can be used to express a Sox2 inhibitor or regeneration agent that is an shRNA, an miRNA, or an shRNA embedded in an miRNA (an shRNA-mir).

Cell type-specific promoters that can be included in the vectors described herein to express a nucleic acid molecule that is or that encodes a Sox2 inhibitor and/or a regeneration agent in one or more inner ear cell types are provided in Table 8, below.

TABLE 8
Inner ear cell type-specific promoters

| Cell Type | Promoter |
|---|---|
| Supporting | Glial Acidic Fibrillary Protein (GFAP), Solute |
| cells | Carrier Family 1 Member 3 (GLAST), Hes Family |
| | BHLH Transcription Factor 1 (HES1), Jagged 1 |
| | (JAG1), Notch 1 (NOTCH1), Leucine Rich Repeat |
| | Containing G Protein-Coupled Receptor 5 (LGR5), |
| | SOX2, Hes Family BHLH Transcription Factor 5 |
| | (HES5), LFNG O-Fucosylpeptide 3-Beta-N- |
| | Acetylglucosaminyltransferase (LFNG), Kringle |
| | Containing Transmembrane Protein 1 |
| | (KREMEN1), Anterior Gradient 3, Protein |
| | Disulphide Isomerase Family Member (AGR3), |
| | SRY-Box 9 (SOX9), and Solute Carrier Family 6 |
| | Member 14 (SLC6A14) |
| Hair | Myosin 15A (MYO15), Growth Factor Independent |
| cells | 1 Transcriptional Repressor (GFI1), POU Class 4 |
| | Homeobox 3 (POU4F3), Myosin 7a (MYO7A) |
| Type II | Calbindin 2 (CALB2), Microtubule associated |
| vestibular | protein tau (MAPT), Annexin A4 (ANXA4), Otoferlin |
| hair cells | (OTOF) |

Exemplary Myo15 promoters are described in International Application Publication Nos. WO2019210181 and WO2020163761A1, and exemplary SLC6A14 promoters are described in International Application No. PCT/US2020/058795, the disclosures of which are incorporated herein by reference.

Once a nucleic acid molecule has been incorporated into the nuclear DNA or into the nucleus of a mammalian cell, the transcription of this nucleic acid molecule can be induced by methods known in the art. For example expression can be induced by exposing the mammalian cell to an external chemical reagent, such as an agent that modulates the binding of a transcription factor and/or RNA polymerase to the mammalian promoter and thus regulates gene expression. The chemical reagent can serve to facilitate the binding of RNA polymerase and/or transcription factors to the mammalian promoter, e.g., by removing a repressor protein that has bound the promoter. Alternatively, the chemical reagent can serve to enhance the affinity of the mammalian promoter for RNA polymerase and/or transcription factors such that the rate of transcription of the gene located downstream of the promoter is increased in the presence of the chemical reagent. Examples of chemical reagents that potentiate polynucleotide transcription by the above mechanisms include tetracycline and doxycycline. These reagents are commercially available (Life Technologies, Carlsbad, Calif.) and can be administered to a mammalian cell in order to promote gene expression according to established protocols. Further control of expression of a Sox2 inhibitor and/or a regeneration agent described herein can be achieved using conditional regulation elements, such as Cre recombinase systems, including FLEx-Cre, as described in Saunders et al., Front Neural Circuits 6:47 (2012).

Other DNA sequence elements that may be included in polynucleotides (e.g., polynucleotides encoding a dominant negative Sox2 protein or Atoh1) for use in the compositions and methods described herein include enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that encode a protein of interest and additionally include a mammalian enhancer sequence. Many enhancer sequences are now known from mammalian genes, and examples include enhancers from the genes that encode mammalian globin, elastase, albumin, α-fetoprotein, and insulin. Enhancers for use in the compositions and methods described herein also include those that are derived from the genetic material of a virus capable of infecting a eukaryotic cell. Examples include the SV40 enhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription include the CMV enhancer and RSV enhancer. An enhancer may be spliced into a vector containing a polynucleotide encoding a protein of interest, for example, at a position 5′ or 3′ to this gene. In a preferred orientation, the enhancer is positioned at the 5′ side of the promoter, which in turn is located 5′ relative to the polynucleotide encoding a protein of interest.

The nucleic acid vectors containing a Sox2 inhibitor and/or a regeneration agent described herein may include a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cell. The addition of the WPRE to a vector can result in a substantial improvement in the level of transgene expression from several different promoters, both in vitro and in vivo.

In some embodiments, the nucleic acid vectors containing a Sox2 inhibitor and/or a regeneration agent described herein include a reporter sequence, which can be useful in verifying the expression of a nucleic acid molecule or encoded protein, for example, in cells and tissues (e.g., in regenerated hair cells, Type II hair cells, or supporting cells). Reporter sequences that may be provided in a transgene include DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements that drive their expression, such as a promoter, the reporter sequences provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA), and immunohistochemistry. For example, where the marker sequence is the LacZ gene, the presence of the vector carrying the signal is detected by assays for β-galactosidase activity. Where the transgene is green fluorescent protein or luciferase, the vector carrying the signal may be measured visually by color or light production in a luminometer.

### Methods for the Delivery of Exogenous Nucleic Acids to Target Cells

Techniques that can be used to introduce a nucleic acid molecule, such as a nucleic acid molecule that is or encodes a Sox2 inhibitor and/or a regeneration agent, into a target cell (e.g., a mammalian cell) are well known in the art. For instance, electroporation can be used to permeabilize mammalian cells (e.g., human target cells) by the application of an electrostatic potential to the cell of interest. Mammalian cells, such as human cells, subjected to an external electric field in this manner are subsequently predisposed to the uptake of exogenous nucleic acids. Electroporation of mammalian cells is described in detail, e.g., in Chu et al., Nucleic Acids Research 15:1311 (1987), the disclosure of which is incorporated herein by reference. A similar technique, Nucleofection™, utilizes an applied electric field in order to stimulate the uptake of exogenous polynucleotides into the nucleus of a eukaryotic cell. Nucleofection™ and protocols useful for performing this technique are described in detail, e.g., in Distler et al., Experimental Dermatology 14:315 (2005), as well as in US 2010/0317114, the disclosures of each of which are incorporated herein by reference.

Additional techniques useful for the transfection of target cells include the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Squeeze-poration is described in detail, e.g., in Sharei et al., Journal of Visualized Experiments 81:e50980 (2013), the disclosure of which is incorporated herein by reference.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for instance, by direct fusion of the liposome with the cell membrane or by endocytosis of the complex. Lipofection is described in detail, for instance, in U.S. Pat. No. 7,442,386, the disclosure of which is incorporated herein by reference. Similar techniques that exploit ionic interactions with the cell membrane to provoke the uptake of foreign nucleic acids include contacting a cell with a cationic polymer-nucleic acid complex. Exemplary cationic molecules that associate with polynucleotides so as to impart a positive charge favorable for interaction with the cell membrane include activated dendrimers (described, e.g., in Dennig, Topics in Current Chemistry 228:227 (2003), the disclosure of which is incorporated herein by reference) polyethylenimine, and diethylaminoethyl (DEAE)-dextran, the use of which as a transfection agent is described in detail, for instance, in Gulick et al., Current Protocols in Molecular Biology 40:1:9.2:9.2.1 (1997), the disclosure of which is incorporated herein by reference. Magnetic beads are another tool that can be used to transfect target cells in a mild and efficient manner, as this methodology utilizes an applied magnetic field in order to direct the uptake of nucleic acids. This technology is described in detail, for instance, in US 2010/0227406, the disclosure of which is incorporated herein by reference.

Another useful tool for inducing the uptake of exogenous nucleic acids by target cells is laserfection, also called optical transfection, a technique that involves exposing a cell to electromagnetic radiation of a particular wavelength in order to gently permeabilize the cells and allow polynucleotides to penetrate the cell membrane. The bioactivity of this technique is similar to, and in some cases found superior to, electroporation.

Impalefection is another technique that can be used to deliver genetic material to target cells. It relies on the use of nanomaterials, such as carbon nanofibers, carbon nanotubes, and nanowires. Needle-like nanostructures are synthesized perpendicular to the surface of a substrate. DNA containing the gene, intended for intracellular delivery, is attached to the nanostructure surface. A chip with arrays of these needles is then pressed against cells or tissue. Cells that are impaled by nanostructures can express the delivered gene(s). An example of this technique is described in Shalek et al., PNAS 107: 1870 (2010), the disclosure of which is incorporated herein by reference.

Magnetofection can also be used to deliver nucleic acids to target cells. The magnetofection principle is to associate nucleic acids with cationic magnetic nanoparticles. The magnetic nanoparticles are made of iron oxide, which is fully biodegradable, and coated with specific cationic proprietary molecules varying upon the applications. Their association with the gene vectors (DNA, siRNA, viral vector, etc.) is achieved by salt-induced colloidal aggregation and electrostatic interaction. The magnetic particles are then concentrated on the target cells by the influence of an external magnetic field generated by magnets. This technique is described in detail in Scherer et al., Gene Therapy 9:102 (2002), the disclosure of which is incorporated herein by reference.

Another useful tool for inducing the uptake of exogenous nucleic acids by target cells is sonoporation, a technique that involves the use of sound (typically ultrasonic frequencies) for modifying the permeability of the cell plasma membrane to permeabilize the cells and allow polynucleotides to penetrate the cell membrane. This technique is described in detail, e.g., in Rhodes et al., Methods in Cell Biology 82:309 (2007), the disclosure of which is incorporated herein by reference.

Microvesicles represent another potential vehicle that can be used to modify the genome of a target cell according to the methods described herein. For instance, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Vectors for Delivery of Exogenous Nucleic Acids to Target Cells

In addition to achieving high rates of transcription and translation, stable expression of an exogenous polynucleotide in a mammalian cell can be achieved by integration of the polynucleotide into the nuclear genome of the mammalian cell. A variety of vectors for the delivery and integration of polynucleotides into the nuclear DNA of a mammalian cell have been developed. Examples of expression vectors are described in, e.g., Gellissen, Production of Recombinant Proteins: Novel Microbial and Eukaryotic Expression Systems (John Wiley & Sons, Marblehead, M A, 2006). Expression vectors for use in the compositions and methods described herein contain a nucleic acid molecule that is or encodes a Sox2 inhibitor and/or a regeneration agent, as well as, e.g., additional sequence elements used for the expression of these agents and/or the integration of these polynucleotide sequences into the genome of a mammalian cell. Vectors that can contain a Sox2 inhibitor and/or a regeneration agent include plasmids (e.g., circular DNA molecules that can autonomously replicate inside a cell), cosmids (e.g., pWE or sCos vectors), artificial chromosomes (e.g., a human artificial chromosome (HAC), a yeast artificial chromosome (YAC), a bacterial artificial chromosome (BAC), or a P1-derived artificial chromosome (PAC)), and viral vectors. Certain vectors that can be used for the expression of a polynucleotide that is or encodes a Sox2 inhibitor and/or a regeneration agent include plasmids that contain regulatory sequences, such as enhancer regions, which direct gene transcription. Other useful vectors for expression of a polynucleotide that is or encodes a Sox2 inhibitor and/or a regeneration agent contain polynucleotide sequences that enhance the rate of translation or improve the stability or nuclear export of the mRNA that results from transcription. These sequence elements include, e.g., 5′ and 3′ untranslated regions, an internal ribosomal entry site (IRES), and polyadenylation signal site in order to direct efficient transcription of the polynucleotide carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

### Viral Vectors for Nucleic Acid Delivery

Viral genomes provide a rich source of vectors that can be used for the efficient delivery of a polynucleotide of interest into the genome of a target cell (e.g., a mammalian cell, such as a human cell). Viral genomes are particularly useful vectors for gene delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, 1996)). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, U.S. Pat. No. 5,801,030, the disclosure of which is incorporated herein by reference as it pertains to viral vectors for use in gene therapy.

### AAV Vectors for Nucleic Acid Delivery

In some embodiments, polynucleotides of the compositions and methods described herein are incorporated into rAAV vectors and/or virions in order to facilitate their introduction into a cell. rAAV vectors useful in the compositions and methods described herein are recombinant nucleic acid constructs that include (1) a promoter, (2) a heterologous nucleic acid molecule that is or encodes a Sox2 inhibitor and/or a regeneration agent, and (3) viral sequences that facilitate stability and expression of the heterologous nucleic acid molecules. The viral sequences may include those sequences of AAV that are required in cis for replication and packaging (e.g., functional ITRs) of the DNA into a virion. Such rAAV vectors may also contain marker or reporter genes. Useful rAAV vectors have one or more of the AAV WT genes deleted in whole or in part, but retain functional flanking ITR sequences. The AAV ITRs may be of any serotype suitable for a particular application. For use in the methods and compositions described herein, the ITRs can be AAV2 ITRs. Methods for using rAAV vectors are described, for example, in Tal et al., J. Biomed. Sci. 7:279 (2000), and Monahan and Samulski, Gene Delivery 7:24 (2000), the disclosures of each of which are incorporated herein by reference as they pertain to AAV vectors for gene delivery.

The polynucleotides and vectors described herein (e.g., a polynucleotide that is or encodes Sox2 inhibitor or a regeneration agent or a vector containing the same) can be incorporated into a rAAV virion in order to facilitate introduction of the polynucleotide or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described, for instance, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8, 9, 10, 11, rh10, rh39, rh43, rh74, AAV2-QuadYF, Anc80, Anc80L65, DJ, DJ/8, DJ/9, 7m8, and PHP (PHP.B, PHP.B2, PHP.B3, PHP.eb, PHP.S, PHP.A). For targeting vestibular hair cells or vestibular supporting cells, AAV1, AAV2, AAV8, AAV9, Anc80, 7m8, DJ, PHP.B, PHP.B2, PHP.B3, PHP.eB, PHP.S, and PHP.A serotypes may be particularly useful. Serotypes evolved for transduction of the retina may also be used in the methods and compositions described herein. Construction and use of AAV vectors and AAV proteins of different serotypes are described, for instance, in Chao et al., Mol. Ther. 2:619 (2000); Davidson et al., Proc. Natl. Acad. Sci. USA 97:3428 (2000); Xiao et al., J. Virol. 72:2224 (1998); Halbert et al., J. Virol. 74:1524 (2000); Halbert et al., J. Virol. 75:6615 (2001); and Auricchio et al., Hum. Molec. Genet. 10:3075 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype (e.g., AAV9) pseudotyped with a capsid gene derived from a serotype other than the given serotype (e.g., AAV1, AAV2, AAV3, AAV4, AAV5, AAV6, AAV7, AAV8, etc.). Techniques involving the construction and use of pseudotyped rAAV virions are known in the art and are described, for instance, in Duan et al., J. Virol. 75:7662 (2001); Halbert et al., J. Virol. 74:1524 (2000); Zolotukhin et al., Methods, 28:158 (2002); and Auricchio et al., Hum. Molec. Genet. 10:3075 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet., 25:436 (2000) and Kolman and Stemmer, Nat. Biotechnol. 19:423 (2001).

### Pharmaceutical Compositions

The Sox2 inhibitors and regeneration agents described herein may be incorporated into a vehicle for administration into a patient, such as a human patient suffering from vestibular dysfunction (e.g., dizziness, vertigo, loss of balance or imbalance, bilateral vestibulopathy (bilateral vestibular hypofunction), oscillopsia, or a balance disorder). Pharmaceutical compositions containing a Sox2 inhibitor and/or a regeneration agent described herein can be prepared using methods known in the art. For example, such compositions can be prepared using, e.g., physiologically acceptable carriers, excipients or stabilizers (Remington: The Science and Practice of Pharmacology 22nd edition, Allen, L. Ed. (2013); incorporated herein by reference), and in a desired form, e.g., in the form of lyophilized formulations or aqueous solutions.

Mixtures of a Sox2 inhibitor and/or a regeneration agent described herein may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated. For local administration to the ear (e.g., the middle or inner ear), the composition may be formulated to contain a synthetic perilymph solution. An exemplary synthetic perilymph solution includes 20-200 mM NaCl, 1-5 mM KCl, 0.1-10 mM CaCl₂), 1-10 mM glucose, and 2-50 mM HEPEs, with a pH between about 6 and 9 and an osmolality of about 300 mOsm/kg. The person responsible for administration will, in any event, determine the appropriate dose for the individual subject. Moreover, for human administration, preparations may meet sterility, pyrogenicity, general safety, and purity standards as required by FDA Office of Biologics standards.

### Methods of Treatment

The compositions described herein may be administered to a subject having or at risk of developing vestibular dysfunction by a variety of routes, such as local administration to the middle or inner ear (e.g., administration into the perilymph or endolymph, such as through the oval window, round window, or semicircular canal (e.g., the horizontal canal), or by transtympanic or intratympanic injection, e.g., administration to a vestibular supporting cell or hair cell), intravenous, parenteral, intradermal, transdermal, intramuscular, intranasal, subcutaneous, percutaneous, intratracheal, intraperitoneal, intraarterial, intravascular, inhalation, perfusion, lavage, and oral administration. The most suitable route for administration in any given case will depend on the particular composition administered, the patient, pharmaceutical formulation methods, administration methods (e.g., administration time and administration route), the patient's age, body weight, sex, severity of the disease being treated, the patient's diet, and the patient's excretion rate. Compositions may be administered once, or more than once (e.g., once annually, twice annually, three times annually, bi-monthly, monthly, or bi-weekly).

Subjects that may be treated as described herein are subjects having or at risk of developing vestibular dysfunction. The compositions and methods described herein can be used to treat subjects having or at risk of developing damage to vestibular hair cells (e.g., damage related to disease or infection, head trauma, ototoxic drugs (e.g., aminoglycosides), or aging), subjects having or at risk of developing vestibular dysfunction (e.g., dizziness, vertigo, loss of balance or imbalance, bilateral vestibulopathy (also called bilateral vestibular hypofunction), oscillopsia, or a balance disorder), subjects carrying a genetic mutation associated with vestibular dysfunction, or subjects with a family history of hereditary vestibular dysfunction. In some embodiments, the disease associated with damage to or loss of hair cells (e.g., vestibular hair cells) is an autoimmune disease or condition in which an autoimmune response contributes to hair cell damage or death. Autoimmune diseases linked to vestibular dysfunction include autoimmune inner ear disease (AIED), polyarteritis nodosa (PAN), Cogan's syndrome, relapsing polychondritis, systemic lupus erythematosus (SLE), Wegener's granulomatosis, Sjögren's syndrome, and Behcet's disease. Some infectious conditions, such as Lyme disease and syphilis can also cause vestibular dysfunction (e.g., by triggering autoantibody production). Viral infections, such as rubella, cytomegalovirus (CMV), lymphocytic choriomeningitis virus (LCMV), HSV types 1&2, West Nile virus (WNV), human immunodeficiency virus (HIV) varicella zoster virus (VZV), measles, and mumps, can also cause vestibular dysfunction. In some embodiments, the subject has vestibular dysfunction that is associated with or results from loss of hair cells (e.g., vestibular hair cells). In some embodiments, compositions and methods described herein can be used to treat a subject having or at risk of developing oscillopsia. In some embodiments, compositions and methods described herein can be used to treat a subject having or at risk of developing bilateral vestibulopathy. In some embodiments, the compositions and methods described herein can be used to treat a subject having or at risk of developing a balance disorder (e.g., imbalance). The subject can be a human adult, adolescent, child, infant, or term newborn (e.g., a subject with a mature vestibular system). The methods described herein may include a step of screening a subject for one or more mutations in genes known to be associated with vestibular dysfunction prior to treatment with or administration of the compositions described herein. A subject can be screened for a genetic mutation using standard methods known to those of skill in the art (e.g., genetic testing). The methods described herein may also include a step of assessing vestibular function in a subject prior to treatment with or administration of the compositions described herein. Vestibular function may be assessed using standard tests, such as eye movement testing (e.g., electronystagmogram (ENG) or videonystagmogram (VNG)), tests of the vestibulo-ocular reflex (VOR) (e.g., the head impulse test (Halmagyi-Curthoys test), which can be performed at the bedside or using a video-head impulse test (VHIT), or the caloric reflex test), posturography, rotary-chair testing, ECOG, vestibular evoked myogenic potentials (VEMP), and specialized clinical balance tests, such as those described in Mancini and Horak, Eur J Phys Rehabil Med, 46:239 (2010). These tests can also be used to assess vestibular function in a subject after treatment with or administration of the compositions described herein. The compositions and methods described herein may also be administered as a preventative treatment to patients at risk of developing vestibular dysfunction, e.g., patients who have a family history of vestibular dysfunction (e.g., inherited vestibular dysfunction), patients carrying a genetic mutation associated with vestibular dysfunction who do not yet exhibit symptoms of vestibular dysfunction, or patients exposed to risk factors for acquired vestibular dysfunction (e.g., disease or infection, head trauma, ototoxic drugs, or aging). The compositions and methods described herein can also be used to treat a subject with idiopathic vestibular dysfunction.

The compositions and methods described herein can be used to induce or increase the generation of Type I vestibular hair cells, to increase the number of Type I vestibular hair cells (e.g., the total number of Type I vestibular hair cells in the vestibular system), and/or to induce or increase hair cell regeneration in a subject (e.g., vestibular hair cell regeneration). In some embodiments, the compositions and methods described herein increase the generation of Type I vestibular hair cells, increase the number of Type I vestibular hair cells, and/or induce or increase hair cell regeneration in the striolar region, in the extrastriolar region, or in both the striolar and extrastriolar regions of the vestibular organs (e.g., the utricle and/or the crista). In some embodiments, the compositions and methods described herein increase the generation of Type I vestibular hair cells, increase the number of Type I vestibular hair cells, and/or induce or increase hair cell regeneration in the crista, in the utricle, in the saccule, in the crista and in the utricle, or in all three of the crista, the utricle, and the saccule. Subjects that may benefit from compositions that promote or increase generation of Type I vestibular hair cells, increase Type I vestibular hair cell numbers, and/or promote or increase vestibular hair cell regeneration include subjects having or at risk of developing vestibular dysfunction as a result of loss of hair cells (e.g., loss of vestibular hair cells related to trauma (e.g., head trauma), disease or infection, ototoxic drugs, or aging), subjects with abnormal vestibular hair cells (e.g., vestibular hair cells that do not function properly compared to normal vestibular hair cells), subjects with damaged vestibular hair cells (e.g., vestibular hair cell damage related to trauma (e.g., head trauma), disease or infection, ototoxic drugs, or aging), or subjects with reduced vestibular hair cell numbers due to genetic mutations or congenital abnormalities.

The compositions and methods described herein can also be used to prevent or reduce vestibular dysfunction caused by ototoxic drug-induced hair cell damage or death (e.g., vestibular hair cell damage or death) in subjects who have been treated with ototoxic drugs, or who are currently undergoing or soon to begin treatment with ototoxic drugs. Ototoxic drugs are toxic to the cells of the inner ear, and can cause vestibular dysfunction (e.g., vertigo, dizziness, loss of balance or imbalance, bilateral vestibulopathy (bilateral vestibular hypofunction), or oscillopsia). Drugs that have been found to be ototoxic include aminoglycoside antibiotics (e.g., gentamycin, neomycin, streptomycin, tobramycin, kanamycin, vancomycin, and amikacin), viomycin, antineoplastic drugs (e.g., platinum-containing chemotherapeutic agents, such as cisplatin, carboplatin, and oxaliplatin), loop diuretics (e.g., ethacrynic acid and furosemide), salicylates (e.g., aspirin, particularly at high doses), and quinine. In some embodiments, the methods and compositions described herein can be used to treat bilateral vestibulopathy (bilateral vestibular hypofunction) or oscillopsia due to aminoglycoside ototoxicity (e.g., generate additional Type I vestibular hair cells to replace damaged or dead cells and/or promote or increase hair cell regeneration in a subject with aminoglycoside-induced bilateral vestibulopathy (bilateral vestibular hypofunction) or oscillopsia).

Treatment may include administration of a composition containing a Sox2 inhibitor and, optionally, a regeneration agent, described herein in various unit doses. Each unit dose will ordinarily contain a predetermined-quantity of the therapeutic composition. The quantity to be administered, and the particular route of administration and formulation, are within the skill of those in the clinical arts. A unit dose need not be administered as a single injection but may comprise continuous infusion over a set period of time. Dosing may be performed using a syringe pump to control infusion rate in order to minimize damage to the inner ear (e.g., the vestibular labyrinth). A composition of the invention may include a dosage of a Sox2 inhibitor of the invention ranging from 0.01 to 500 mg/kg (e.g., 0.01, 0.1, 0.2, 0.3, 0.4, 0.5, 1, 2, 3, 4, 5, 10, 15, 20, 25, 30, 35, 40, 45, 50, 100, 150, 200, 250, 300, 350, 400, 450, or 500 mg/kg) and, in a more specific embodiment, about 0.1 to about 30 mg/kg and, in a more specific embodiment, about 0.3 to about 30 mg/kg. The dosage may be adapted by the physician in accordance with conventional factors such as the extent of the disease and different parameters of the subject. In cases in which the Sox2 inhibitor and/or regeneration agent is administered using an AAV vector (e.g., an AAV vector containing an siRNA, an shRNA or miRNA targeting Sox2 mRNA or a Sox2 promoter, a component of a gene editing system targeting Sox2, a polynucleotide encoding a dominant negative Sox2 protein, or a polynucleotide encoding Atoh1, e.g., an AAV1, AAV2, AAV3, AAV4, AAV5, AAV6, AAV7, AAV8, AAV9, AAV10, AAV11, rh10, rh39, rh43, rh74, AAV2-QuadYF, Anc80, Anc80L65, DJ, DJ/8, DJ/9, 7m8, PHP.B, PHP.B2, PBP.B3, PHP.A, PHP.eb, or PHP.S vector), the viral vector may be administered to the patient at a dose of, for example, from about 1×10⁹ vector genomes (VG)/mL to about 1×10¹⁶ VG/mL (e.g., 1×10⁹ VG/mL, 2×10⁹ VG/mL, 3×10⁹ VG/mL, 4×10⁹ VG/mL, 5×10⁹ VG/mL, 6×10⁹ VG/mL, 7×10⁹ VG/mL, 8×10⁹ VG/mL, 9×10⁹ VG/mL, 1×10¹⁰ VG/mL, 2×10¹⁰ VG/mL, 3×10¹⁰ VG/mL, 4×10¹⁰ VG/mL, 5×10¹⁰ VG/mL, 6×10¹⁰ VG/mL, 7×10¹⁰ VG/mL, 8×10¹⁰ VG/mL, 9×10¹⁰ VG/mL, 1×10¹¹ VG/mL, 2×10¹¹ VG/mL, 3×10¹¹ VG/mL, 4×10¹¹ VG/mL, 5×10¹¹ VG/mL, 6×10¹¹ VG/mL, 7×10¹¹ VG/mL, 8×10¹¹ VG/mL, 9×10¹¹ VG/mL, 1×10¹² VG/mL, 2×10¹² VG/mL, 3×10¹² VG/mL, 4×10¹² VG/mL, 5×10¹² VG/mL, 6×10¹² VG/mL, 7×10¹² VG/mL, 8×10¹² VG/mL, 9×10¹² VG/mL, 1×10¹³ VG/mL, 2×10¹³ VG/mL, 3×10¹³ VG/mL, 4×10¹³ VG/mL, 5×10¹³ VG/mL, 6×10¹³ VG/mL, 7×10¹³ VG/mL, 8×10¹³ VG/mL, 9×10¹³ VG/mL, 1×10¹⁴ VG/mL, 2×10¹⁴ VG/mL, 3×10¹⁴ VG/mL, 4×10¹⁴ VG/mL, 5×10¹⁴ VG/mL, 6×10¹⁴ VG/mL, 7×10¹⁴ VG/mL, 8×10¹⁴ VG/mL, 9×10¹⁴ VG/mL, 1×10¹⁵ VG/mL, 2×10¹⁵ VG/mL, 3×10¹⁵ VG/mL, 4×10¹⁵ VG/mL, 5×10¹⁵ VG/mL, 6×10¹⁵ VG/mL, 7×10¹⁵ VG/mL, 8×10¹⁵ VG/mL, 9×10¹⁵ VG/mL, or 1×10¹⁶ VG/mL) in a volume of 1 μL to 200 μL (e.g., 1, 2, 3, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200 μL). The AAV vector may be administered to the subject at a dose of about 1×10⁷ VG/ear to about 2×10¹⁵ VG/ear (e.g., 1×10⁷ VG/ear, 2×10⁷ VG/ear, 3×10⁷ VG/ear, 4×10⁷ VG/ear, 5×10⁷ VG/ear, 6×10⁷ VG/ear, 7×10⁷ VG/ear, 8×10⁷ VG/ear, 9×10⁷ VG/ear, 1×10⁸ VG/ear, 2×10⁸ VG/ear, 3×10⁸ VG/ear, 4×10⁸ VG/ear, 5×10⁸ VG/ear, 6×10⁸ VG/ear, 7×10⁸ VG/ear, 8×10⁸ VG/ear, 9×10⁸ VG/ear, 1×10⁹ VG/ear, 2×10⁹ VG/ear, 3×10⁹ VG/ear, 4×10⁹ VG/ear, 5×10⁹ VG/ear, 6×10⁹ VG/ear, 7×10⁹ VG/ear, 8×10⁹ VG/ear, 9×10⁹ VG/ear, 1×10¹⁰ VG/ear, 2×10¹⁰ VG/ear, 3×10¹⁰ VG/ear, 4×10¹⁰ VG/ear, 5×10¹⁰ VG/ear, 6×10¹⁰ VG/ear, 7×10¹⁰ VG/ear, 8×10¹⁰ VG/ear, 9×10¹⁰ VG/ear, 1×10¹¹ VG/ear, 2×10¹¹ VG/ear, 3×10¹¹ VG/ear, 4×10¹¹ VG/ear, 5×10¹¹ VG/ear, 6×10¹¹ VG/ear, 7×10¹¹ VG/ear, 8×10¹¹ VG/ear, 9×10¹¹ VG/ear, 1×10¹² VG/ear, 2×10¹² VG/ear, 3×10¹² VG/ear, 4×10¹² VG/ear, 5×10¹² VG/ear, 6×10¹² VG/ear, 7×10¹² VG/ear, 8×10¹² VG/ear, 9×10¹² VG/ear, 1×10¹³ VG/ear, 2×10¹³ VG/ear, 3×10¹³ VG/ear, 4×10¹³ VG/ear, 5×10¹³ VG/ear, 6×10¹³ VG/ear, 7×10¹³ VG/ear, 8×10¹³ VG/ear, 9×10¹³ VG/ear, 1×10¹⁴ VG/ear, 2×10¹⁴ VG/ear, 3×10¹⁴ VG/ear, 4×10¹⁴ VG/ear, 5×10¹⁴ VG/ear, 6×10¹⁴ VG/ear, 7×10¹⁴ VG/ear, 8×10¹⁴ VG/ear, 9×10¹⁴ VG/ear, 1×10¹⁵ VG/ear, or 2×10¹⁵ VG/ear).

The compositions described herein are administered in an amount sufficient to improve vestibular function (e.g., improve balance or reduce dizziness or vertigo), treat bilateral vestibulopathy (bilateral vestibular hypofunction), treat oscillopsia, treat a balance disorder, increase the generation of Type I vestibular hair cells, increase the number of Type I vestibular hair cells, or promote or increase regeneration of vestibular hair cells. Vestibular function may be evaluated using standard tests for balance and vertigo (e.g., eye movement testing (e.g., ENG or VNG), VOR testing (e.g., head impulse testing (Halmagyi-Curthoys testing, e.g., VHIT), or caloric reflex testing), posturography, rotary-chair testing, ECOG, VEMP, and specialized clinical balance tests) and may be improved by 5% or more (e.g., 5%, 10%, 15%, 20%, 30%, 40%, 50%, 60%, 70%, 80%, 90%, 100%, 125%, 150%, 200% or more) compared to measurements obtained prior to treatment. The compositions described herein may also be administered in an amount sufficient to slow or prevent the development or progression of vestibular dysfunction (e.g., in subjects who carry a genetic mutation associated with vestibular dysfunction, who have a family history of vestibular dysfunction (e.g., hereditary vestibular dysfunction), or who have been exposed to risk factors associated with vestibular dysfunction (e.g., ototoxic drugs, head trauma, or disease or infection) but who do not exhibit vestibular dysfunction (e.g., vertigo, dizziness, imbalance, bilateral vestibulopathy (bilateral vestibular hypofunction), oscillopsia, or a balance disorder), or in subjects exhibiting mild to moderate vestibular dysfunction). Type I vestibular hair cell generation or numbers, or hair cell regeneration may be evaluated indirectly based on tests of vestibular function, and may be increased by 5% or more (e.g., 5%, 10%, 15%, 20%, 30%, 40%, 50%, 60%, 70%, 80%, 90%, 100%, 125%, 150%, 200% or more) compared to Type I vestibular hair cell generation or numbers, or hair cell regeneration prior to administration of a composition described herein or compared to an untreated subject. These effects may occur, for example, within 1 week, 2 weeks, 3 weeks, 4 weeks, 5 weeks, 6 weeks, 7 weeks, 8 weeks, 9 weeks, 10 weeks, 15 weeks, 20 weeks, 25 weeks, or more, following administration of the compositions described herein. The patient may be evaluated 1 month, 2 months, 3 months, 4 months, 5 months, 6 months or more following administration of the composition depending on the dose and route of administration used for treatment. Depending on the outcome of the evaluation, the patient may receive additional treatments.

### Kits

The compositions described herein can be provided in a kit for use in treating vestibular dysfunction. Compositions may include a Sox2 inhibitor described herein and may further include a regeneration agent (e.g., an agent that increases Atoh1 expression and/or a Notch inhibitor). The kit can further include a package insert that instructs a user of the kit, such as a physician, to perform the methods described herein. The kit may optionally include a syringe or other device for administering the composition.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1—Establishment of a Mouse Model for Sox2 Knockout in Adult Vestibular Hair Cells

To demonstrate that knockdown of Sox2 in vestibular type II hair cells results in their conversion to type I hair cells, we first established a model system to achieve knockout of Sox2 in adult vestibular hair cells in vivo. To achieve targeted knockout of Sox2 in adult mice we relied on cre-lox recombination technology and Sox2^{fl/fl} mice (Sox2^{tm1.1Lan}) in which cre-mediated recombination of the LoxP sites flanking the Sox2 gene in the Sox2^{fl/fl} mouse will result in deletion of the Sox2 gene. A plasmid containing an expression cassette encoding a mouse Myo15 promoter driving expression of a Cre recombinase transgene was packaged into AAV8 at a titer of 3.2×10¹³ vg/mL. The AAV8.Myo15.Cre was administered locally to naïve adult Sox2^{fl/fl} mice (Sox2^{tm1.1Lan}) or C57Bl6 mice via posterior semicircular canal (intra-labyrinth (IL)) delivery at a dose of 3.2×10¹⁰ vg/ear. One month after AAV8.Myo15.Cre delivery to the Sox2^{fl/fl} or C57Bl6 mice, the animals were sacrificed, and the vestibular tissue was collected for single-cell RNA sequencing (scRNAseq) or immunohistochemistry (IHC).

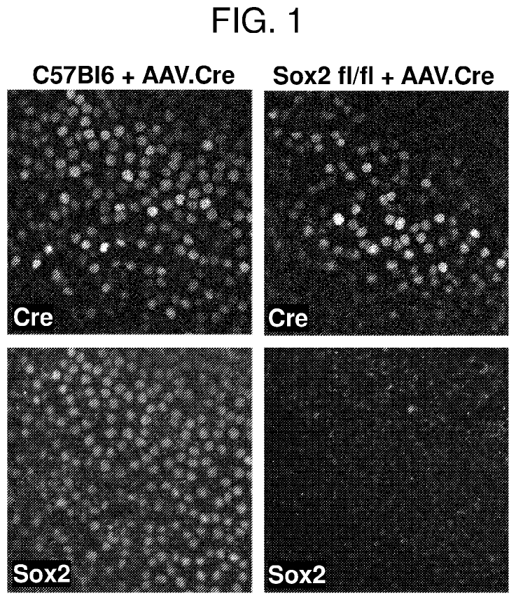

For IHC, the vestibular tissue was fixed for one hour at room temperature with 4% paraformaldehyde. The tissue was washed with phosphate buffered saline (PBS) three times for five minutes, then blocked with 10% serum in PBS+0.5% Triton X-100 (PBST) for 3 hours at room temperature followed by overnight incubation at 4° C. in primary rabbit anti-Sox2 antibody (1:500 dilution; Cat #ab97959, Abcam, Cambridge, Mass.) or primary mouse monoclonal anti-Cre antibody (1:100 dilution; Cat #C7988, Sigma-Aldrich, St. Louis, Mo.) in PBST plus 2% serum. Tissues were brought to room temperature and then washed three times for five minutes with PBS. Tissues were then incubated with Alexa Fluor 568 donkey anti-mouse or Alexa Fluor 488 anti-rabbit secondary antibodies (1:500 dilution; ThermoFisher Scientific, Waltham Mass.) in PBST plus 2% serum for three hours at room temperature. Organs were washed three times for five minutes with PBS, mounted onto glass slides, and confocal images were obtained using the Zeiss LSM 880 with airyscan (Zeiss, Germany). Sox2 protein expression was significantly reduced in vestibular hair cells expressing Cre in the Sox2^{fl/fl} mice compared to C57Bl6 mice (FIG. 1). These data validate our model system for Sox2 knockout in adult mouse vestibular type hair cells.

### Example 2—AAV8 Transduces Type II but not Type I Hair Cells of the Vestibular Organs

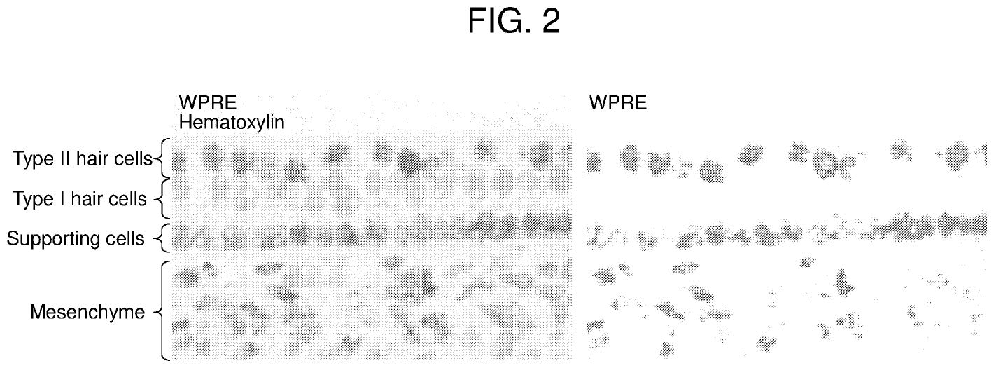

To determine relative transduction of AAV into type I vs. type II hair cells of the vestibular organs, an AAV8 virus was delivered by IL injection into adult mice at 9.78×10⁹ vg/ear. After two weeks, whole ears were fixed, decalcified, paraffin-embedded, and sectioned with hematoxylin staining to visualize cross-sections of whole tissues and nuclei. Sections were probed for the Woodchuck Hepatitis Virus Posttranscriptional Regulatory Element (WPRE) element of the AAV vector genome by DNAscope. The AAV vector genomes were detected in the vestibular mesenchymal cells, supporting cells, and type II hair cells (FIG. 2). No AAV vector genomes were detected in vestibular type I hair cells (FIG. 2). These data indicate that AAV8 transduces vestibular type II but not type I hair cells in the adult mouse in vivo.

### Example 3—Examination of Morphological and Molecular Changes in Vestibular Type II Hair Cells after Sox2 Knockout (Converting Type II Hair Cells) in Naive Mice In Vivo

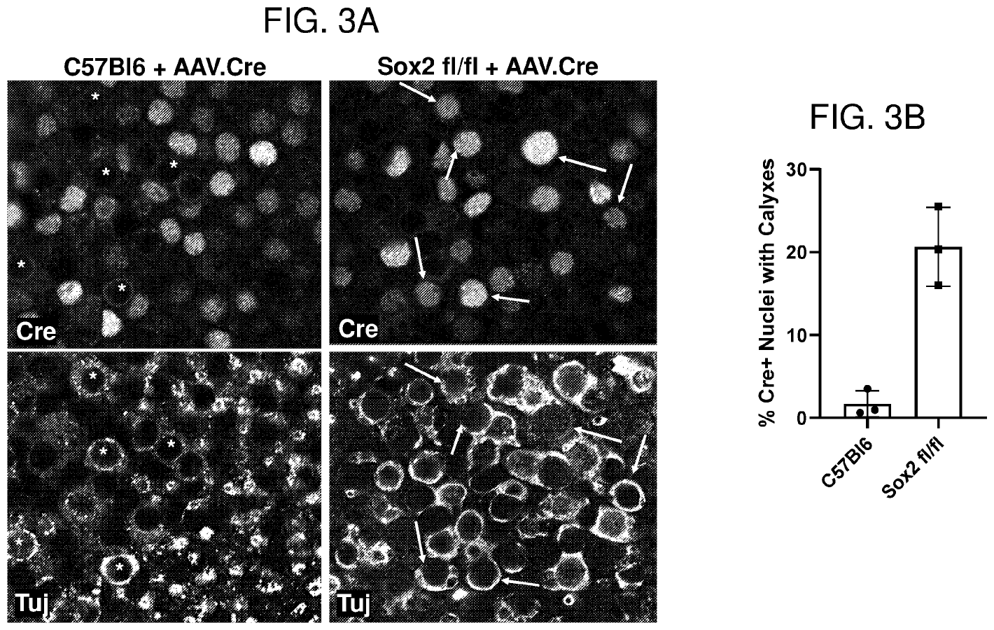

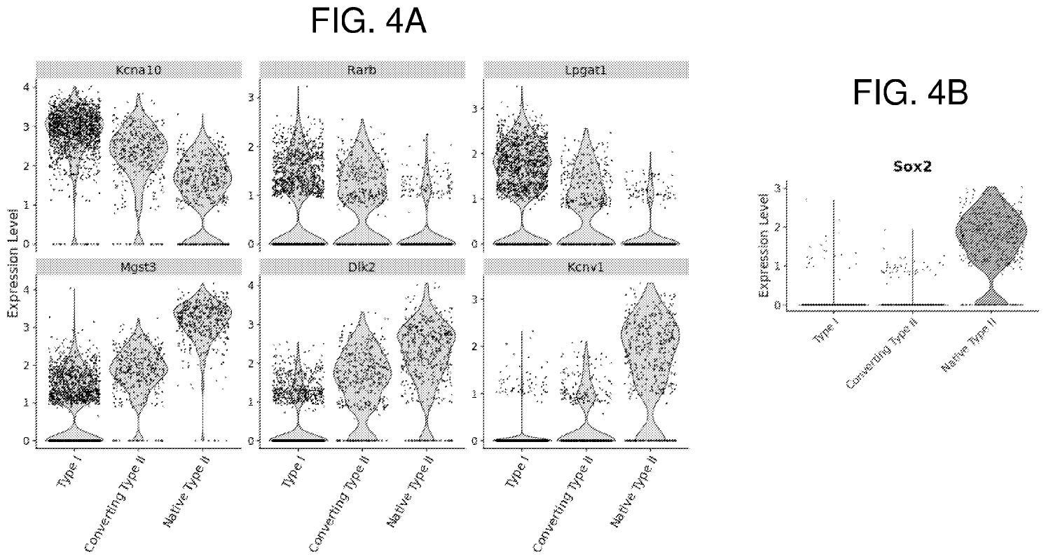

To determine if knockout of Sox2 in vestibular type II hair cells results in their conversion to type I hair cells we utilized the Sox2^{fl/fl} model system to achieve knockout of Sox2 in adult vestibular hair cells in vivo. The AAV8.Myo15.Cre virus was administered locally to adult naïve Sox2^{fl/fl} mice (Sox2^{tm1.1Lan}) or C57Bl6 mice via posterior semicircular canal (intra-labyrinth (IL)) delivery at a dose of 3.2×10¹⁰ vg/ear. One month after AAV8.Myo15.Cre delivery to the Sox2^{fl/fl} or C57Bl6 mice, the animals were sacrificed, and the vestibular tissue was collected for immunohistochemistry (IHC) or scRNAseq. For IHC, the temporal bones were removed, the utricles were microdissected out, and fluorescence immunolabelling for the neuronal marker Tuj (primary rabbit anti-Tubulin-3 (Tuj) antibody 1:500 dilution; Cat #802001, BioLegend, San Diego, Calif.) and Cre marker (primary mouse monoclonal anti-Cre antibody 1:100 dilution; Cat #C7988, Sigma-Aldrich, St. Louis, Mo.) was performed. Organs were washed three times for five minutes with PBS, mounted onto glass slides, and confocal images were obtained using the Zeiss LSM 880 with airyscan (Zeiss, Germany). Hair cell nuclei positive for Cre adopted significantly more calyxes (a type of neuronal afferent unique to vestibular type I hair cells) in the Sox2^{fl/fl} mice compared with C57Bl6 mice (FIGS. 3A-3B). For scRNAseq, vestibular tissue was dissociated, and single cells were captured and prepared for single-cell RNA-Seq with a 10× Genomics Chromium system. Sequencing was performed on an Illumina NovaSeq, reads were aligned with CellRanger, and downstream analysis was performed with Seurat. To identify cell clusters, dimensionality reduction was performed on the variable genes using principal component analysis, followed by Louvain graph-based clustering. Gene expression and clustering was visualized in a Uniform Manifold Approximation and Projection (UMAP). Unsupervised clustering partitioned hair cells into 3 groups labeled as native type II hair cells, type I hair cells and converting type II hair cells based on a representative selection of transcripts used to define type 1 (FIG. 4A top row; Kcna10, Rarb, Lpgat1) and type II (FIG. 4A bottom row; Mgst3, DIk2, Kcnv1) hair cells. Examination of transcripts by scRNAseq indicated that Sox2 mRNA was significantly reduced in a subpopulation of type II hair cells, referred to hereafter as converting type II hair cells, in the Sox2^{fl/fl} mice (FIG. 4B). Converting type II hair cells from the Sox2^{fl/fl} mice showed increased expression of transcripts typically associated with type 1 hair cells (FIG. 4A; top row) and a decreased expression of transcripts typically associated with type II hair cells (FIG. 4A; bottom row). Taken together, these results suggest that converting type II hair cells are acquiring morphological and molecular features that are more like type 1 hair cells when Sox2 expression is inhibited.

### Example 4—Examination of Morphological and Molecular Changes in Vestibular Type II Hair Cells after Sox2 Knockout in a Mouse IDPN Damage Model In Vivo

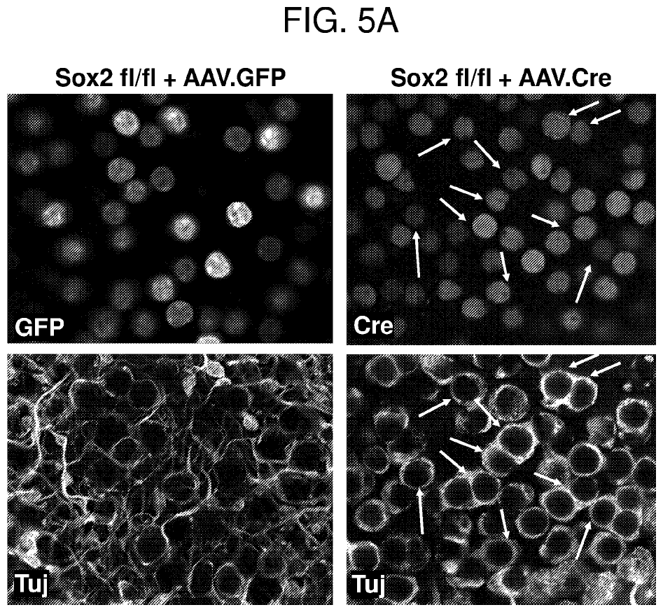

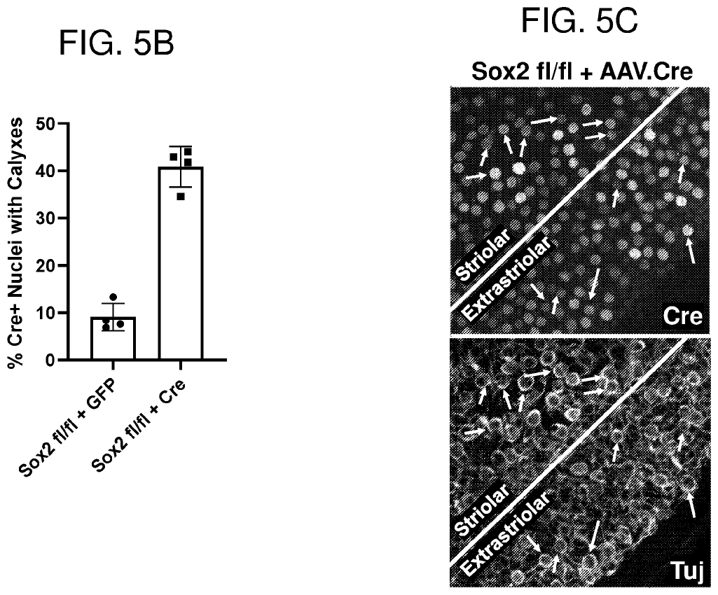

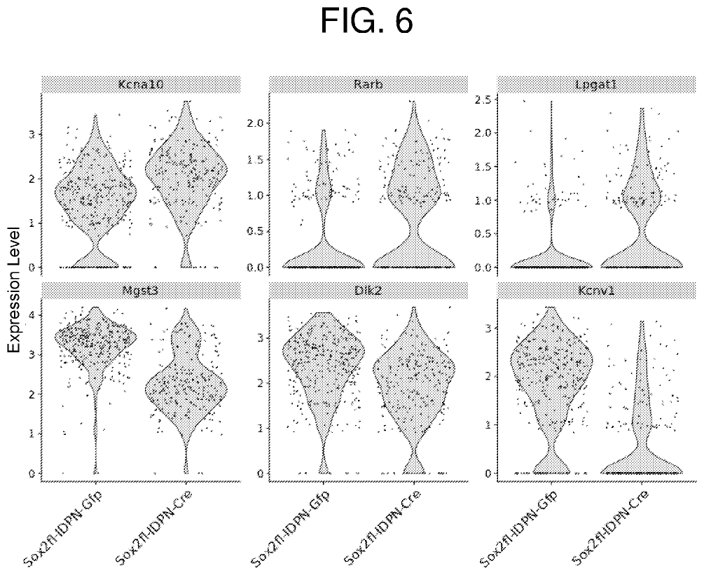

Next, we assessed if Sox2 knockout in surviving type II hair cells after IDPN-induced damage would result in the conversion of those cells to type I-like hair cells. To lesion hair cells, adult Sox2^{fl/fl} mice were weighed and injected intraperitoneally with 4 mg/kg sterile 3,3′-iminodipropionitrile (IDPN; TCI America, 10010) in PBS. IDPN preferentially damages type I hair cells over type II hair cells. A plasmid containing an expression cassette encoding the mouse Myo15 promoter driving expression of a nuclear-targeted green fluorescent protein (GFP fused to the H2B fragment of the histone 2b gene) was packaged into AAV8 at a titer of 2.42×10¹³ vg/mL. Two weeks after IDPN injection, AAV8.Myo15.Cre (left ears) and AAV8.Myo15.GFP (right ears) were administered into the posterior semicircular canal (n=6) at a dose of 3.2×10¹⁰ vg/ear (AAV8.Myo15.Cre) or 2.42×10¹⁰ vg/ear (AAV8.Myo15.GFP). One month after virus delivery to the Sox2″ mice, the animals were sacrificed, and the vestibular tissue was collected for immunohistochemistry (INC) or scRNAseq. For IHC, the temporal bones were removed, the utricles were microdissected out, and fluorescence immunolabelling for the neuronal marker Tuj (primary rabbit anti-Tubulin-3 (Tuj) antibody 1:500 dilution; Cat #802001, BioLegend, San Diego, Calif.) and Cre marker (primary mouse monoclonal anti-Cre antibody 1:100 dilution; Cat #C7988, Sigma-Aldrich, St. Louis, Mo.) was performed. Organs were washed three times for five minutes with PBS, mounted onto glass slides, and confocal images were obtained using the Zeiss LSM 880 with airyscan (Zeiss, Germany). Hair cell nuclei positive for Cre adopted significantly more calyxes (a type of neuronal afferent unique to vestibular type I hair cells) in the Sox2^{fl/fl} ears treated with AAV8.Myo15.Cre compared to ears treated with AAV8.Myo15.GFP (FIGS. 5A-5B). This phenotype was not restricted to a particular region within the sensory epithelium as calyx adoption by Cre positive nuclei was observed in the striolar and extrastriolar regions of the utricle (FIG. 5C). For scRNAseq, the cristae were collected, the cells were dissociated, and single cells were captured for scRNAseq. Downstream analysis of the scRNAseq data was performed and a representative selection of transcripts used to define type I (FIG. 6; top row; Kcna10, Rarb, Lpgat1) and type II (FIG. 6; bottom row; Mgst3, DIk2, Kcnv1) hair cells were selected from the data. Converting type II hair cells from the Sox2^{fl/fl} mice showed increased expression of transcripts typically associated with type I hair cells (FIG. 6; top row) and a decreased expression of transcripts typically associated with type II hair cells (FIG. 6; bottom row). Taken together, these results suggest that vestibular type II hair cells are capable acquiring morphological and molecular features that are more like type I hair cells in the context of damage.

### Example 5—Examination of Molecular Changes in Regenerating and Pre-Existing Vestibular Hair Cells after Sox2 Knockout in a Mouse IDPN Damage Model In Vivo

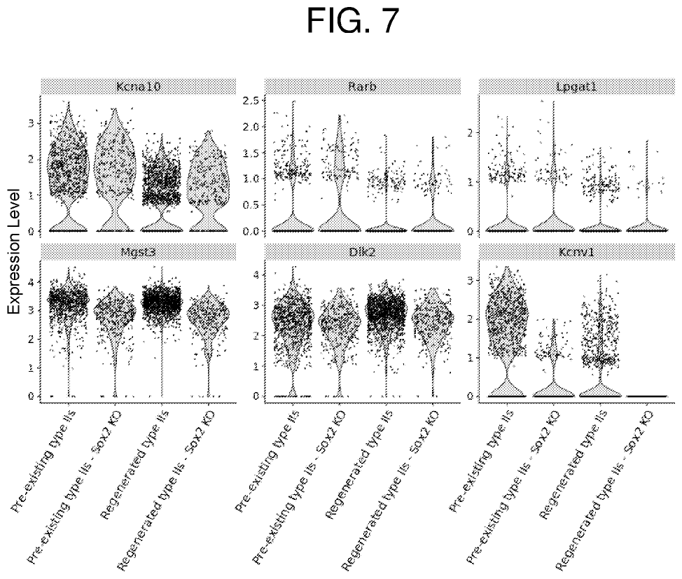

Using our IDPN-damage model, we examined if knockout of Sox2 in regenerating vestibular hair cells resulted in the regeneration of type I-like vestibular hair cells. To lesion hair cells, adult Sox2^{fl/fl} mice were weighed and injected intraperitoneally with 4 mg/kg sterile 3,3′-iminodipropionitrile (IDPN; TCI America, 10010) in PBS. A plasmid containing an expression cassette encoding a GFAP promoter driving expression of a human ATOH1 and co-expressing a nuclear-targeted green fluorescent protein (GFP fused to the H2B fragment of the histone 2b gene) was packaged into AAV8 at a titer of 2.47×10¹³ vg/mL. Two weeks after IDPN injection, AAV8.GFAP.ATOH1+/−AAV8.Myo15.Cre were administered into the posterior semicircular canal (n=6) of C57Bl6 and Sox2^{fl/fl} at a dose of 2.42×10¹⁰ vg/ear (AAV8.GFAP.ATOH1) or 3.2×10¹⁰ vg/ear (AAV8.Myo15.Cre). One month after virus delivery, the animals were sacrificed, and the vestibular tissue was collected for scRNAseq. For scRNAseq, the cristae were collected, the cells were dissociated, and single cells were captured for scRNAseq. Downstream analysis of the scRNAseq data was performed and a representative selection of transcripts used to define type I (FIG. 7; Kcna10, Rarb, Lpgat1) and type II (FIG. 7; Mgst3, DIk2, Kcnv1) hair cells were selected from the data. Delivery of AAV8.GFAP.ATOH1 alone into the vestibular system of the Sox2″ mice resulted in the regeneration of type II-like vestibular hair cells (FIG. 7; regenerated type IIs) whereas co-delivery of AAV8.GFAP.ATOH1 and AAV8.Myo15.Cre resulted in the regeneration of vestibular hair cells with increased expression of transcripts typically associated with type 1 hair cells (FIG. 7; top row; Regenerated type IIs—Sox2 KO) and decreased expression of transcripts typically associated with type II hair cells (FIG. 7; bottom row; regenerated type IIs—Sox2 KO). Taken together, these results suggest that knockout of Sox2 combined with regeneration of vestibular hair cells via ATOH1 overexpression after damage leads to the generation of type I like hair cells. Notably, co-delivery of AAV8.GFAP.ATOH1 and AAV8.Myo15.Cre also resulted in pre-existing type II hair cells increasing their expression of type 1 hair cell transcripts and decreasing their expression of type II hair cell transcripts (FIG. 7; top and bottom row, respectively; pre-existing type IIs—Sox2 KO).

### Example 6—Examination of Sox2 Dysregulation and Downregulation by RNAi and Dominant-Negative Over-Expression

In order to knockdown endogenous Sox2 activity we tested three modalities: AAV-shRNA, AAV-dominant-negative-Sox2 (dnSox2), and siRNA.

### AA VshRNA

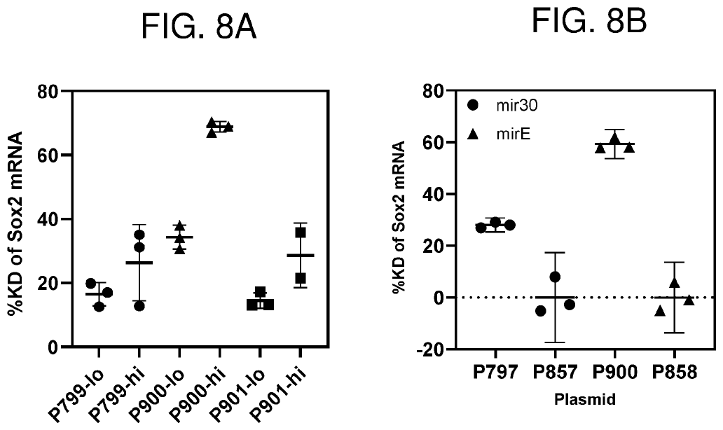

AAV8-CMV-shRNA transfer plasmids contained a single Sox2 shRNA hairpin driven by a CMV promoter and scaffolded by miRNAs mir30 or mir-E and co-expressing GFP as a marker. We transfected P19 cells with plasmids containing shRNA candidate Sox2 _2 in a mir30 scaffold (P797; SEQ ID NO: 30), Sox2_2 in a mirE scaffold (P900; SEQ ID NO: 31), Sox2_4 in a mir30 scaffold (P799; SEQ ID NO: 32), Sox2_4 in mirE scaffold (P901; SEQ ID NO: 33), and non-targeting controls (NT). After 3 days, we sorted GFP+ cells that were successfully transfected into populations with high and lower expression of GFP, extracted RNA, and performed RT-qPCR to assess Sox2 knockdown. Sox2 expression across all groups was normalized to the endogenous control gene, Actb and the percentage of Sox2 knockdown compared to non-targeting controls. We observed significantly more knockdown of Sox2 for cells in populations with greater GFP expression (P799-hi, P900-hi, P901-hi), confirming that knockdown was correlated with the degree of plasmid expression. Furthermore, we observed significantly greater knockdown of Sox2 mRNA for cells expressing shSox2_2 with the mirE backbone (P900) compared to the those expressing shSox2_4 with the mirE backbone (P901) (FIG. 8A). In a separate experiment we sorted cells with the highest GFP expression and compared cells expressing the same shRNA sequence (shSox2_2), but with two different backbones, shSox2_2 on mir30 (P797) and shSox2_2 on mirE (P900). We see that while both shRNAs show knockdown compared to their scrambled controls (P857 and P858), shSox2_2 in the mirE backbone (P900) results in two-fold better Sox2 knockdown compared to the same sequence in the mir30 backbone (P797) (FIG. 8B). We believe that a mirE backbone might provide superior Sox2 shRNA knockdown activity as compared to a mir30 backbone.

siRNA

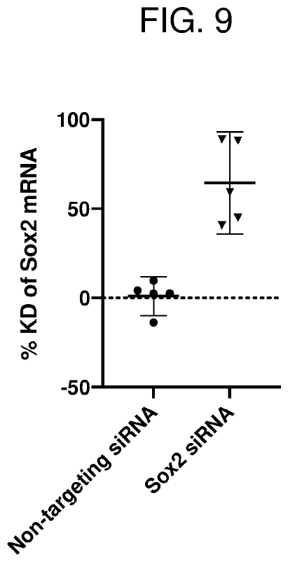

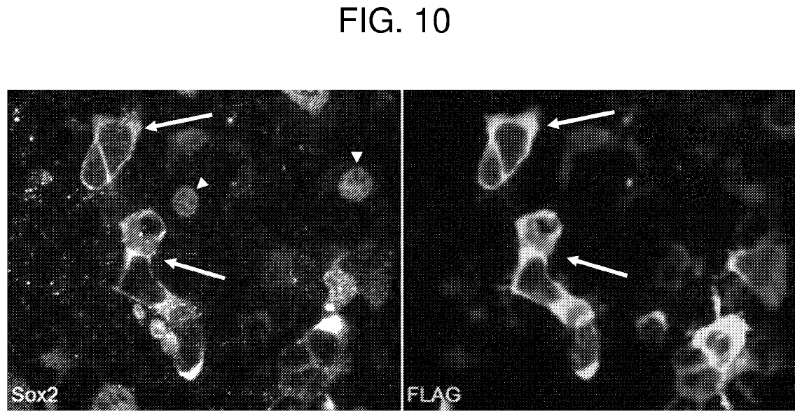

To assess the potential for siRNA knockdown of endogenous Sox2 in the mouse vestibule we excised utricles from adult C57BL6/J mice and cultured them in media with siRNAs. Adult utricle explants were cultured for 72 hours in cell culture media (Accell siRNA Delivery Media, Horizon Discovery with N₂ and SM1 supplements) with a pool of siRNAs (SEQ ID NOs: 35 and 36, 37 and 38, 39 and 40, and 41 and 42; in which each pair represents a sense and an anti-sense strand targeting SEQ ID NOs: 25, 11, 26, and 27, respectively) or non-targeting siRNAs at a concentration of 1 μM (FIG. 9). After 72 hours we extracted RNA from the utricle sensory epithelia (n=3 utricle per condition) and performed RT-qPCR to assess Sox2 knockdown in these utricles. We observed up to ˜80% knockdown (KD) in the utricle sensory epithelia.

dnSox2

The AAV8-CMV-dnSox2 (SEQ ID NO: 34)-FLAG transfer plasmid encodes a CMV promoter-driven, nuclear localization mutant (NLS) form of Sox2 (NM_011443.4) followed by a glycine linker to connect a FLAG epitope tag. The NLS mutation is predicted to prevent dnSOX2 localization to nucleus, but not prevent it from binding endogenous SOX2 or its transcriptional cofactors. Thus, dnSOX2 may act as a dominant negative by sequestering proteins required for SOX2 transcriptional activity in the cytoplasm. AAV8-CMV-dnSox2-FLAG was transfected into P19 cells to examine its effects on endogenous SOX2 localization. Cells were fixed and immunostained for SOX2 (1:500; #ab97959; Abcam) and FLAG (1:1000; F1804; Sigma) three days after transfection. FIG. 10 shows a field containing P19 cells that express native Sox2 in their nuclei (arrowheads) and a separate set of cells that co-expressed native SOX2 and dnSOX2-FLAG (arrows). In these cells, the native SOX2 is redistributed from the nucleus to the cytoplasm and corresponds to the localization of the dnSOX2-FLAG species (FIG. 10; arrows). Together, these data indicate that when dnSOX2 is co-expressed with endogenous SOX2, the former prevents the latter from entering the nucleus and, therefore, limits SOX2 transcriptional activity.

### Example 7—Examination of Distinct Molecular Differences in Adult Vs. Embryonic/Early Postnatal Type II Vestibular Hair Cells of Naïve Mice

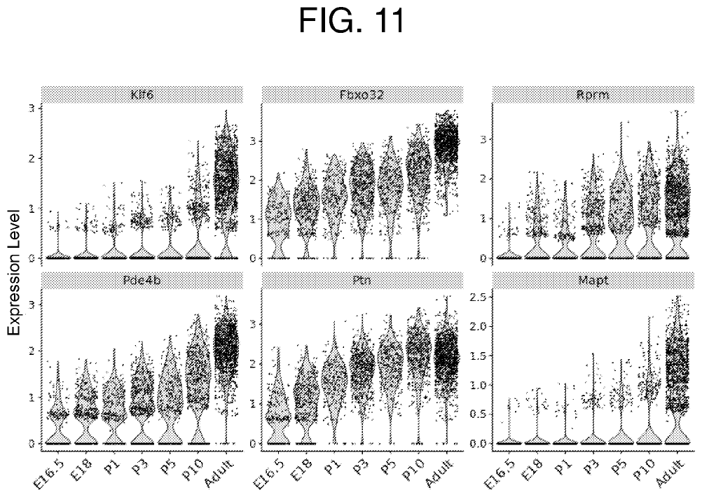

The effect of Sox2 knockout on conversion of adult type II to type I hair cells could not have been predicted based on earlier work on embryonic hair cells because adult type II hair cells are molecularly distinct from embryonic/early postnatal type II hair cells. To demonstrate this conversion in adults, vestibular organs were dissected from naïve CD1 mice at various embryonic (E) and post-natal (P) stages (E16.5, E18, P1, P3, P5, P10, and adult (>P50)), the cells were dissociated, and single cells were captured for scRNAseq. Downstream analysis of the scRNAseq data was performed and a representative selection of transcripts were chosen that were either not expressed or expressed at low levels in embryonic/early postnatal type II hair cells and expressed highly at adult ages (FIG. 11). These data indicate that adult type II hair cells are molecularly distinct and therefore different than embryonic/early postnatal type II vestibular hair cells.

### Example 8—Examination of Type I and Type II Vestibular Hair Cell Markers after Retinoic Acid Pathway Modulation in Adult Vs. Embryonic/Early Postnatal Mice

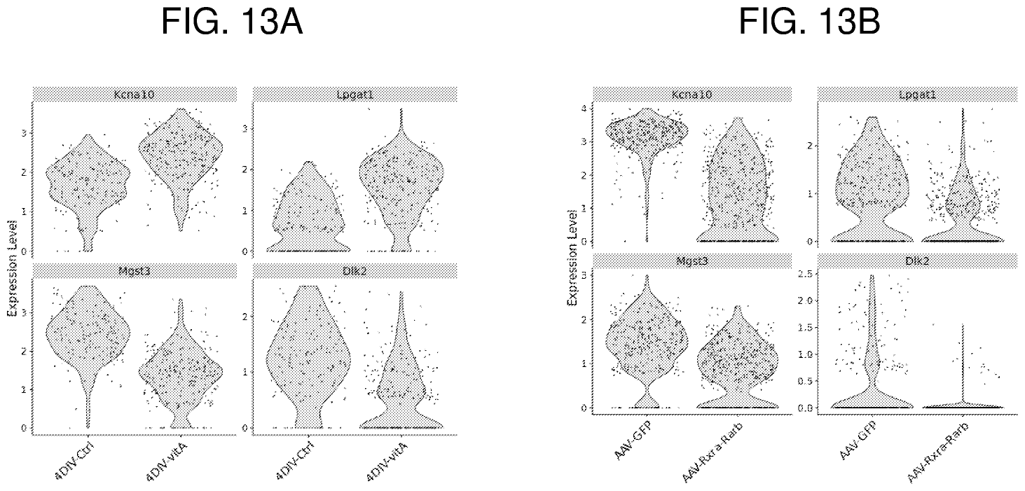

Analysis of vestibular hair cell development suggests that the retinoic acid pathway may play a significant role in determination of type I hair cell fate. To evaluate the potential for this pathway to modulate type I hair cell fate, we dissected and cultured embryonic utricles from E15.5 CD1 mice in the presence or absence of 0.1 μM vitamin A (retinoic acid precursor). After 4 days in culture the cells were dissociated, and single cells were captured for scRNAseq. Downstream analysis of the scRNAseq data was performed and representative transcripts used to define type I hair cells (Kcna10 and Lpgat1) and type II hair cells (Mgst3 and DIk2) were selected from the data. Addition of vitamin A to the culture media of embryonic utricles resulted in the development of hair cells with increased expression of transcripts typically associated with type I hair cells (FIG. 13A; top row) and decreased expression of transcripts typically associated with type II hair cells (FIG. 13A; bottom row).

Next, we wanted to determine if we could drive the conversion of vestibular type II hair cells to type I hair cells in adult utricle explant cultures via similar modulation of the retinoic acid pathway. From our scRNAseq data, it is evident that downstream receptors of the retinoic acid pathway, namely Rarb and Rxra, are not expressed in adult type II hair cells. Therefore, we designed two plasmids containing an expression cassette encoding the cytomegalovirus (CMV) promoter driving expression of either the mouse retinoic acid receptor Rarb or the mouse retinoic acid receptor Rxra. These plasmids were packaged into AAV8 at a titer of 1.24×10¹³ vg/mL (AAV8.CMV.Rarb) or 9.12×10¹² vg/mL (AAV8.CMV.Rxra). As a control, a plasmid containing an expression cassette encoding the CMV promoter driving expression of a nuclear-targeted green fluorescent protein (GFP fused to the H2B fragment of the histone 2b gene) was packaged into AAV8 at a titer of 4.43×10¹³ vg/mL. Utricle explants were dissected from adult mice and were cultured with both the AAV8.CMV.Rarb and AAV8.CMV.Rxra viruses together or with the AAV8.CMV.GFP virus alone. In all cases, 1 μM retinoic acid was included in the culture media. After 3 days, the viruses were washed out of the media and the utricles were cultured for an additional 6 days in the presence of 1 μM retinoic acid. After the culture period, the cells were dissociated, and single cells were captured for scRNAseq. Downstream analysis of the scRNAseq data was performed and the same representative transcripts used to define type I hair cells in FIG. 13A were selected from the data.

Data from the adult cultures indicated that, in contrast to embryonic hair cells, activation of the retinoic acid pathway via addition of retinoic acid to the culture media did not result in the generation of hair cells with increased expression of transcripts typically associated with type I hair cells (FIG. 13B; top row) and decreased expression of transcripts typically associated with type II hair cells (FIG. 13B; bottom row).

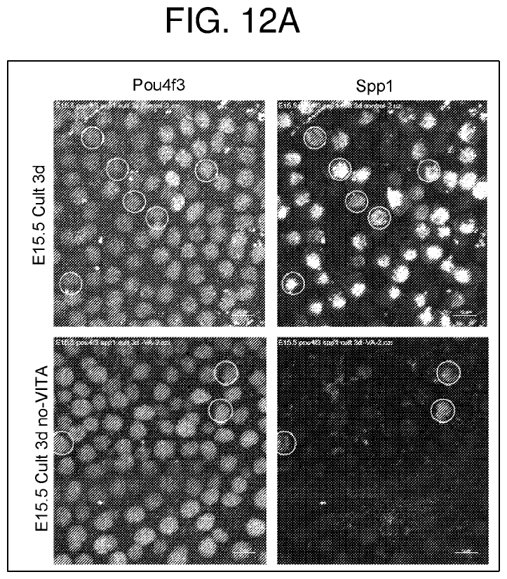

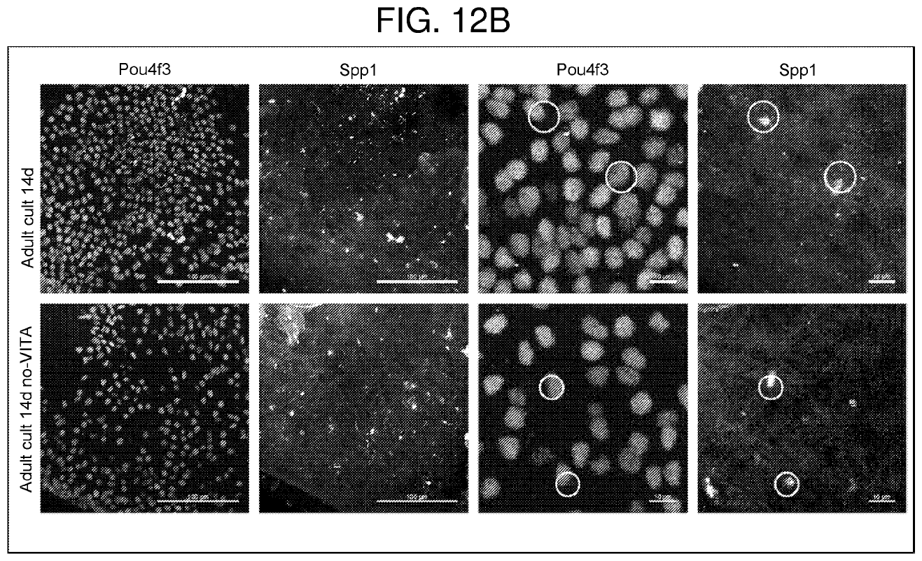

To further explore these differences of the effect of the retinoic acid pathway on embryonic and adult hair cells, we compared the effects of 0.1 μM vitamin A on cultured utricles from embryonic and adult animals. We looked at the effect of vitamin A on the induction of SPP1 (a Type I HC marker) and found that this agent dramatically increased the number of SPP1-positive cells in embryonic, but not adult tissue (FIGS. 12A-12B).

The results observed in embryonic cells were very different from those observed in adult cells. This suggest that perturbations that drive embryonic/early postnatal vestibular hair cell fate toward the type I hair cell path do not predict efficacy of those same perturbations generating type I hair cells in adults. These data add emphasis to Example 7, in which we confirm that embryonic/early postnatal and adult vestibular hair cells are molecularly distinct and therefore cannot be expected to respond similarly to the same molecular perturbations.

### Example 9—Administration of a Composition Containing a Sox2 Inhibitor to a Subject with Vestibular Dysfunction

According to the methods disclosed herein, a physician of skill in the art can treat a patient, such as a human patient, with vestibular dysfunction (e.g., vertigo, dizziness, loss of balance or imbalance, bilateral vestibulopathy (bilateral vestibular hypofunction), oscillopsia, or a balance disorder) so as to improve or restore vestibular function. To this end, a physician of skill in the art can administer to the human patient a composition containing a Sox2 inhibitor (e.g., an inhibitory RNA molecule targeting the Sox2 mRNA or a Sox2 promoter, a component of a gene editing system targeting Sox2, or a dominant negative Sox2 protein). The Sox2 inhibitor may be delivered using a viral vector, such as an AAV vector (e.g., an AAV1, AAV2, AAV3, AAV4, AAV5, AAV6, AAV7, AAV8, AAV9, AAV10, AAV11, rh10, rh39, rh43, rh74, AAV2-QuadYF, Anc80, Anc80L65, DJ, DJ/8, DJ/9, 7m8, PHP.B, PHP.B2, PBP.B3, PHP.A, PHP.eb, or PHP.S vector). The composition containing the Sox2 inhibitor may be administered to the patient, for example, by local administration to the inner ear (e.g., injection into the semicircular canal), to treat vestibular dysfunction. If the vestibular dysfunction is thought to result from vestibular hair cell loss, the physician can administer to the human patient a composition containing a regeneration agent (e.g., an agent that increases Atoh1 expression, such as an AAV vector containing a polynucleotide encoding Atoh1 operably linked to a supporting cell promoter (e.g., an SLC6A14 promoter), and/or a Notch inhibitor, such as a small molecule Notch inhibitor (e.g., a gamma-secretase inhibitor) or an inhibitory RNA molecule targeting Notch). If the Sox2 inhibitor and/or regeneration agent is administered using a viral vector, e.g., an AAV vector, the viral vector may be administered to the patient at a dose of, for example, from about 1×10¹⁰ vector genomes (VG) to 1×10¹⁵ VG (e.g., 1×10¹⁰ VG, 2×10¹⁰ VG, 3×10¹⁰ VG, 4×10¹⁰ VG, 5×10¹⁰ VG, 6×10¹⁰ VG, 7×10¹⁰ VG, 8×10¹⁰ VG, 9×10¹⁰ VG, 1×10¹¹ VG, 2×10¹¹ VG, 3×10¹¹ VG, 4×10¹¹ VG, 5×10¹¹ VG, 6×10¹¹ VG, 7×10¹¹ VG, 8×10¹¹ VG, 9×10¹¹ VG, 1×10¹² VG, 2×10¹² VG, 3×10¹² VG, 4×10¹² VG, 5×10¹² VG, 6×10¹² VG, 7×10¹² VG, 8×10¹² VG, 9×10¹² VG, 1×10¹³ VG, 2×10¹³ VG, 3×10¹³ VG, 4×10¹³ VG, 5×10¹³ VG, 6×10¹³ VG, 7×10¹³ VG, 8×10¹³ VG, 9×10¹³ VG, 1×10¹⁴ VG, 2×10¹⁴ VG, 3×10¹⁴ VG, 4×10¹⁴ VG, 5×10¹⁴ VG, 6×10¹⁴ VG, 7×10¹⁴ VG, 8×10¹⁴ VG, 9×10¹⁴ VG, 1×10¹⁵ VG) in a volume of 1 μL to 200 μL (e.g., 1, 2, 3, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200 μL).

Following administration of the composition to a patient, a practitioner of skill in the art can monitor the patient's improvement in response to the therapy by a variety of methods. For example, a physician can monitor the patient's vestibular function by performing standard tests such as electronystagmography, video nystagmography, VOR tests (e.g., head impulse tests (Halmagyi-Curthoys test, e.g., VHIT), or caloric reflex tests), rotation tests, vestibular evoked myogenic potential, or computerized dynamic posturography. A finding that the patient exhibits improved vestibular function in one or more of the tests following administration of the composition compared to test results obtained prior to administration of the composition indicates that the patient is responding favorably to the treatment. Subsequent doses can be determined and administered as needed.

Exemplary embodiments of the invention are described in the enumerated paragraphs below.
E1. A method of generating Type I vestibular hair cells in a human subject in need thereof, comprising administering to the subject an effective amount of a Sox2 inhibitor.
E2. The method of E1, wherein the subject has or is at risk of developing vestibular dysfunction.
E3. A method of treating a subject having or at risk of developing vestibular dysfunction, comprising administering to the subject an effective amount of a Sox2 inhibitor.
E4. The method of any one of E1-E3, wherein the Sox2 inhibitor is an inhibitory RNA molecule targeting Sox2 or a Sox2 promoter, a component of a gene editing system targeting Sox2, a polynucleotide encoding a component of a gene editing system targeting Sox2, a dominant negative Sox2 protein, or a polynucleotide encoding a dominant negative Sox2 protein.
E5. The method of E4, wherein the Sox2 inhibitor is an inhibitory RNA molecule targeting Sox2.
E6. The method of E5, wherein the inhibitory RNA molecule is a short interfering RNA (siRNA).
E7. The method of E5, wherein the inhibitory RNA molecule is a short hairpin RNA (shRNA).
E8. The method of E6 or E7, wherein the siRNA or shRNA targeting Sox2 has a nucleobase sequence containing a portion of at least 8 contiguous nucleobases having at least 80% complementarity to an equal length portion of a target region of an mRNA transcript of a human or murine SOX2 gene.
E9. The method of E8, wherein the target region is an mRNA transcript of the human SOX2 gene.
E10. The method of E8, wherein the target region is at least 8 to 21 contiguous nucleobases of any one of SEQ ID NOs: 5-23, at least 8 to 22 contiguous nucleobases of SEQ ID NO: 28 or SEQ ID NO: 29, or at least 8 to 19 contiguous nucleobases of any one of SEQ ID NOs: 25-27.
E11. The method of E8, wherein the siRNA or shRNA has a nucleobase sequence containing a portion of at least 8 contiguous nucleobases having at least 70% complementarity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity) complementarity to an equal length portion of any one of SEQ ID NOs: 5-23 and 25-29.
E12. The method of E11, wherein the shRNA has a nucleobase sequence having at least 70% complementarity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity) complementarity to any one of SEQ ID NO: 11, SEQ ID NO: 25, SEQ ID NO: 26, SEQ ID NO: 27, SEQ ID NO: 28, and SEQ ID NO: 29.
E13. The method of E11, wherein the shRNA comprises the sequence of nucleotides 2234-2296 of SEQ ID NO: 30 or nucleotides 2234-2296 of SEQ ID NO: 32.
E14. The method of any one of E7-E13, wherein the shRNA is embedded in a microRNA (miRNA) backbone.
E15. The method of E14, wherein the shRNA is embedded in a miR-30 or mir-E backbone.
E16. The method of E15, wherein the shRNA comprises the sequence of nucleotides 2109-2426 of SEQ ID NO: 30, nucleotides 2109-2408 of SEQ ID NO: 31, nucleotides 2109-2426 of SEQ ID NO: 32, or nucleotides 2109-2408 of SEQ ID NO: 33.
E17. The method of E8 or E10, wherein the siRNA comprises a sense strand and an antisense strand selected from the following pairs: SEQ ID NO: 35 and SEQ ID NO: 36; SEQ ID NO: 37 and SEQ ID NO: 38; SEQ ID NO: 39 and SEQ ID NO: 40; and SEQ ID NO: 41 and SEQ ID NO: 42
E18. The method of E5, wherein the inhibitory RNA is an miRNA.
E19. The method of E18, wherein the miRNA is human miR-145, miR-126, miR-200c, miR-429, miR-200b, miR-140, miR-9, miR-21, miR-590, miR-182, or miR-638, or murine miR-134, miR-200c, miR-429, miR-200b, miR-34a, or miR-9.
E20. The method of E4, wherein the Sox2 inhibitor is an inhibitory RNA molecule targeting a Sox2 promoter.
E21. The method of E20, wherein the inhibitory RNA molecule is an miRNA.
E22. The method of E4, wherein the Sox2 inhibitor is a component of a gene editing system targeting Sox2 or a polynucleotide encoding a component of a gene editing system targeting Sox2.
E23. The method of E22, wherein the gene editing system is a zinc finger nuclease (ZFN) system, a transcription activator-like effector-based nuclease (TALEN) system, or a clustered regulatory interspaced short palindromic repeat (CRISPR) system.
E24. The method of E4, wherein the Sox2 inhibitor is a dominant negative Sox2 protein or a polynucleotide encoding a dominant negative Sox2 protein.
E25. The method of E24, wherein the polynucleotide encoding the dominant negative Sox2 protein has the sequence of SEQ ID NO: 24 or SEQ ID NO: 34.
E26. The method of E24, wherein the dominant negative Sox2 protein is a Sox2 protein that lacks most or all of the high mobility group domain (HMGD), a Sox2 protein in which the nuclear localization signals in the HMGD are mutated, a Sox2 protein in which the HMGD is fused to an engrailed repressor domain, or a c-terminally truncated Sox2 protein comprising only the DNA binding domain.
E27. The method of any one of E1-E26, wherein the method further comprises administering a regeneration agent.
E28. The method of E27, wherein the regeneration agent is administered before the Sox2 inhibitor.
E29. The method of E27, wherein the regeneration agent is administered after the Sox2 inhibitor.
E30. The method of E27, wherein the regeneration agent is administered concurrently with the Sox2 inhibitor.
E31. The method of any one of E21-E24, wherein the regeneration agent is an agent that increases Atoh1 expression and/or a Notch inhibitor.
E32. The method of E31, wherein the regeneration agent is an agent that increases Atoh1 expression.
E33. The method of E31 or E32, wherein the agent that increases Atoh1 expression is a polynucleotide encoding Atoh1 (e.g., a polynucleotide encoding SEQ ID NO: 43, such as a polynucleotide having the sequence of SEQ ID NO: 44).
E34. The method of E31 or E32, wherein the agent that increases Atoh1 expression is a small molecule.
E35. The method of E31, wherein the regeneration agent is a Notch inhibitor.
E36. The method of E31 or E35, wherein the Notch inhibitor is an inhibitory RNA targeting Notch, a small molecule Notch inhibitor, an anti-Notch antibody, or a polynucleotide encoding an anti-Notch antibody.
E37. The method of any one of E31, E35, and E36, wherein the Notch inhibitor is an inhibitory RNA targeting Notch.
E38. The method of E36 or E37, wherein the inhibitory RNA targeting Notch is an siRNA, an shRNA, or an miRNA.
E39. The method of any one of E31, E35, and E36, wherein the Notch inhibitor is a small molecule Notch inhibitor.
E40. The method of any one of E31, E35, and E36, wherein the Notch inhibitor is an anti-Notch antibody or a polynucleotide encoding an anti-Notch antibody.
E41. The method of any one of E27-33, E35-E38, and E40, wherein the regeneration agent is administered using a nucleic acid vector.
E42. The method of E41, wherein the nucleic acid vector comprises a promoter operably linked to the regeneration agent.
E43. The method of E42, wherein the regeneration agent is a polynucleotide encoding Atoh1, an siRNA targeting Notch, an shRNA targeting Notch, an miRNA targeting Notch, or a polynucleotide encoding an anti-Notch antibody and the promoter is a pol II promoter.
E44. The method of E43, wherein the pol II promoter is a supporting cell promoter.
E45. The method of E44, wherein the supporting cell promoter is a Glial Acidic Fibrillary Protein (GFAP) promoter, a Solute Carrier Family 1 Member 3 (GLAST) promoter, a Hes Family BHLH Transcription Factor 1 (HES1) promoter, a Jagged 1 (JAG1) promoter, a Notch 1 (NOTCH1) promoter, a Leucine Rich Repeat Containing G Protein-Coupled Receptor 5 (LGR5) promoter, a SOX2 promoter, a Hes Family BHLH Transcription Factor 5 (HESS) promoter, a LFNG 0-Fucosylpeptide 3-Beta-N-Acetylglucosaminyltransferase (LFNG) promoter, a Kringle Containing Transmembrane Protein 1 (KREMEN1) promoter, an Anterior Gradient 3, Protein Disulphide Isomerase Family Member (AGR3) promoter, a SRY-Box 9 (SOX9), or a Solute Carrier Family 6 Member 14 (SLC6A14) promoter.
E46. The method of E42, wherein the regeneration agent is an siRNA targeting Notch, an shRNA targeting Notch, or an miRNA targeting Notch and the promoter is a pol III promoter.
E47. The method of any one of E1-E46, wherein the Sox2 inhibitor is administered using a nucleic acid vector.
E48. The method of E47, wherein the Sox2 inhibitor is an siRNA targeting Sox2 or a Sox2 promoter, an shRNA targeting Sox 2 or a Sox2 promoter, an shRNA targeting Sox2 or a Sox2 promoter embedded in an miRNA, an miRNA targeting Sox2, or an miRNA targeting a Sox2 promoter and the promoter is a pol III promoter.
E49. The method of E46 or E48, wherein the pol III promoter is a ubiquitous pol III promoter.
E50. The method of E49, wherein the ubiquitous pol III promoter is a U6 promoter, an H1 promoter, or a 7SK promoter.
E51. The method of E47, wherein the Sox2 inhibitor is an siRNA targeting Sox2 or a Sox2 promoter, an shRNA targeting Sox2 or a Sox2 promoter, an shRNA targeting Sox2 or a Sox2 promoter embedded in an miRNA, an miRNA targeting Sox2, an miRNA targeting a Sox2 promoter, a polynucleotide encoding a component of a gene editing system targeting Sox2, or a polynucleotide encoding a dominant negative Sox2 protein and the promoter is a pol II promoter.
E52. The method of E43 or E51, wherein the pol II promoter is a ubiquitous promoter.
E53. The method of E52, wherein the ubiquitous pol II promoter is a CMV promoter, a CAG promoter, or a smCBA promoter.
E54. The method of E51, wherein the pol II promoter is a hair cell promoter.
E55. The method of E54, wherein the hair cell promoter is a Myosin 15A (MYO15) promoter, a Growth Factor Independent 1 Transcriptional Repressor (GFI1) promoter, a POU Class 4 Homeobox 3 (POU4F3) promoter, or Myosin 7a (MYO7A) promoter.
E56. The method of E51, wherein the pol II promoter is a Type II vestibular hair cell promoter.
E57. The method of E56, wherein the Type II vestibular hair cell promoter is a Calbindin 2 (CALB2) promoter, a Microtubule associated protein tau (MAPT) promoter, an Annexin A4 (ANXA4) promoter, or an Otoferlin (OTOF) promoter.
E58. The method of any one of E41-E57, wherein the Sox2 inhibitor and the regeneration agent are administered using separate nucleic acid vectors.
E59. The method of any one of E41-57, wherein the Sox2 inhibitor and the regeneration agent are administered using a single nucleic acid vector that expresses both the Sox2 inhibitor and the regeneration agent.
E60. The method of E59, wherein the Sox2 inhibitor and the regeneration agent are expressed using two different promoters.
E61. The method of E59, wherein the Sox2 inhibitor and the regeneration agent are expressed using the same promoter.
E62. The method of any one of E27-E61, wherein the regeneration agent is a polynucleotide encoding Atoh1.
E63. The method of any one of E41-E62, wherein the nucleic acid vector is a plasmid, cosmid, artificial chromosome, or viral vector.
E64. The method of E63, wherein the nucleic acid vector is a viral vector.
E65. The method of E64, wherein the viral vector is selected from the group consisting of an adeno-associated virus (AAV), an adenovirus, and a lentivirus.
E66. The method of E65, wherein the viral vector is an AAV vector.
E67. The method of E66, wherein the AAV vector has an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ, DJ/8, DJ/9, 7m8, PHP.B, PHP.B2, PBP.B3, PHP.A, PHP.eb, and PHP.S.
E68. The method of any one of E2-E67, wherein the vestibular dysfunction comprises vertigo, dizziness, loss of balance (imbalance), bilateral vestibulopathy (bilateral vestibular hypofunction), oscillopsia, or a balance disorder.
E69. The method of any one of E2-E68, wherein the vestibular dysfunction is age-related vestibular dysfunction, head trauma-related vestibular dysfunction, disease or infection-related vestibular dysfunction, or ototoxic drug-induced vestibular dysfunction.
E70. The method of any one of E2-E68, wherein the vestibular dysfunction is associated with a genetic mutation.
E71. The method of E69, wherein the ototoxic drug is an aminoglycoside, an antineoplastic drug, ethacrynic acid, furosemide, a salicylate, or quinine.
E72. The method of any one of E1-E71, wherein the method further comprises evaluating the vestibular function of the subject prior to administering the nucleic acid vector or composition.
E73. The method of any one of E1-E71, wherein the method further comprises evaluating the vestibular function of the subject after administering the nucleic acid vector or composition.
E74. The method of any one of E1-E73, wherein the Sox2 inhibitor and/or regeneration agent is locally administered.
E75. The method of E74, wherein the Sox2 inhibitor and/or regeneration agent is administered to a semicircular canal (intra-labyrinth delivery).
E76. The method of any one of E1-E75, wherein the Sox2 inhibitor and/or regeneration agent is administered in an amount sufficient to prevent or reduce vestibular dysfunction, delay the development of vestibular dysfunction, slow the progression of vestibular dysfunction, improve vestibular function, improve balance, reduce dizziness, reduce vertigo, increase Type I vestibular hair cell numbers, increase the generation of Type I vestibular hair cells, or promote or increase hair cell regeneration.
E77. A nucleic acid vector comprising a Sox2 inhibitor operably linked to a promoter.
E78. The nucleic acid vector of E77, wherein the Sox2 inhibitor is an inhibitory RNA molecule targeting Sox2 or a Sox2 promoter, a polynucleotide encoding a component of a gene editing system targeting Sox2, or a polynucleotide encoding a dominant negative Sox2 protein.
E79. The nucleic acid vector of E78, wherein the Sox2 inhibitor is an inhibitory RNA molecule targeting Sox2.
E80. The nucleic acid vector of E78 or E79, wherein the inhibitory RNA molecule is a short interfering RNA (siRNA).
E81. The nucleic acid vector of E78 or E79, wherein the inhibitory RNA molecule is a short hairpin RNA (shRNA).
E82. The nucleic acid vector of E80 or E81, wherein the siRNA or shRNA targeting Sox2 has a nucleobase sequence containing a portion of at least 8 contiguous nucleobases having at least 80% complementarity to an equal length portion of a target region of an mRNA transcript of a human or murine SOX2 gene.
E83. The nucleic acid vector of E82, wherein the target region is an mRNA transcript of the human SOX2 gene.
E84. The nucleic acid vector of E82, wherein the target region of the siRNA or shRNA is at least 8 to 21 contiguous nucleobases of any one of SEQ ID NOs: 5-23, 28, and 29, or at least 8 to 19 contiguous nucleobases of any one of SEQ ID NOs: 25-27.
E85. The nucleic acid vector of E82, wherein the siRNA or shRNA has a nucleobase sequence containing a portion of at least 8 contiguous nucleobases having at least 70% complementarity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity) complementarity to an equal length portion of any one of SEQ ID NOs: 5-23 and 25-29.
E86. The nucleic acid vector of E85, wherein the shRNA has a nucleobase sequence having at least 70% complementarity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity) complementarity to any one of SEQ ID NO: 11, SEQ ID NO: 25, SEQ ID NO: 26, SEQ ID NO: 27, SEQ ID NO: 28, and SEQ ID NO: 29.
E87. The nucleic acid vector of E85, wherein the shRNA comprises the sequence of nucleotides 2234-2296 of SEQ ID NO: 30 or nucleotides 2234-2296 of SEQ ID NO: 32.
E88. The nucleic acid vector of any one of E81-E87, wherein the shRNA is embedded in a microRNA (miRNA) backbone.
E89. The nucleic acid vector of E78, wherein the shRNA is embedded in a miR-30 or mir-E backbone.
E90. The nucleic acid vector of E89, wherein the shRNA comprises the sequence of nucleotides 2109-2426 of SEQ ID NO: 30, nucleotides 2109-2408 of SEQ ID NO: 31, nucleotides 2109-2426 of SEQ ID NO: 32, or nucleotides 2109-2408 of SEQ ID NO: 33.
E91. The nucleic acid vector of E82 or E84, wherein the siRNA comprises a sense strand and an antisense strand selected from the following pairs: SEQ ID NO: 35 and SEQ ID NO: 36; SEQ ID NO: 37 and SEQ ID NO: 38; SEQ ID NO: 39 and SEQ ID NO: 40; and SEQ ID NO: 41 and SEQ ID NO: 42
E92. The nucleic acid vector of E78 or E79, wherein the inhibitory RNA is an miRNA.
E93. The nucleic acid vector of E92, wherein the miRNA is human miR-145, miR-126, miR-200c, miR-429, miR-200b, miR-140, miR-9, miR-21, miR-590, miR-182, or miR-638, or murine miR-134, miR-200c, miR-429, miR-200b, miR-34a, or miR-9.
E94. The nucleic acid vector of E78, wherein the Sox2 inhibitor is an inhibitory RNA molecule targeting a Sox2 promoter.
E95. The nucleic acid vector of E94, wherein the inhibitory RNA molecule is an miRNA.
E96. The nucleic acid vector of E78, wherein the Sox2 inhibitor is a polynucleotide encoding a component of a gene editing system targeting Sox2.
E97. The nucleic acid vector of E96, wherein the gene editing system is a zinc finger nuclease (ZFN) system, a transcription activator-like effector-based nuclease (TALEN) system, or a clustered regulatory interspaced short palindromic repeat (CRISPR) system.
E98. The nucleic acid vector of E78, wherein the Sox2 inhibitor is a polynucleotide encoding a dominant negative Sox2 protein.
E99. The nucleic acid vector of E98, wherein the polynucleotide encoding the dominant negative Sox2 protein has the sequence of SEQ ID NO: 24 or SEQ ID NO: 34.
E100. The nucleic acid vector of E98, wherein the polynucleotide encoding the dominant negative Sox2 protein is a polynucleotide that encodes a Sox2 protein that lacks most or all of the high mobility group domain (HMGD), a polynucleotide that encodes a Sox2 protein in which the nuclear localization signals in the HMGD are mutated, a polynucleotide that encodes a Sox2 protein in which the HMGD is fused to an engrailed repressor domain, or a polynucleotide that encodes a c-terminally truncated Sox2 protein comprising only the DNA binding domain.
E101. The nucleic acid vector of any one of E77-E100, wherein the nucleic acid vector further comprises a regeneration agent.
E102. The nucleic acid vector of E101, wherein the Sox2 inhibitor and the regeneration agent are expressed using the same promoter.
E103. The nucleic acid vector of E101, wherein the Sox2 inhibitor and the regeneration agent are expressed using different promoters.
E104. The nucleic acid vector of any one of E101-E103, wherein the regeneration agent is an agent that increases Atoh1 expression and/or a Notch inhibitor.
E105. The nucleic acid vector of any one of E104, wherein the regeneration agent is an agent that increases Atoh1 expression.
E106. The nucleic acid vector of E104 or E105, wherein the agent that increases Atoh1 expression is a polynucleotide encoding Atoh1.
E107. The nucleic acid vector of any one of E104, wherein the regeneration agent is a Notch inhibitor.
E108. The nucleic acid vector of E104 or E107, wherein the Notch inhibitor is a polynucleotide encoding an anti-Notch antibody.
E109. The nucleic acid vector of E104 or E107, wherein the Notch inhibitor is an inhibitory RNA targeting Notch.
E110. The nucleic acid vector of E109, wherein the inhibitory RNA targeting Notch is an siRNA.
E111. The nucleic acid vector of E109, wherein the inhibitory RNA targeting Notch is an shRNA.
E112. The nucleic acid vector of E109, wherein the inhibitory RNA targeting Notch is an miRNA.
E113. The nucleic acid vector of any one of E77-E112, wherein the promoter is a pol II promoter.
E114. The nucleic acid vector of E113, wherein the pol II promoter is a supporting cell promoter.
E115. The nucleic acid vector of E114, wherein the supporting cell promoter is a Glial Acidic Fibrillary Protein (GFAP) promoter, a Solute Carrier Family 1 Member 3 (GLAST) promoter, a Hes Family BHLH Transcription Factor 1 (HES1) promoter, a Jagged 1 (JAG1) promoter, a Notch 1 (NOTCH1) promoter, a Leucine Rich Repeat Containing G Protein-Coupled Receptor 5 (LGR5) promoter, a SOX2 promoter, a Hes Family BHLH Transcription Factor 5 (HESS) promoter, a LFNG 0-Fucosylpeptide 3-Beta-N-Acetylglucosaminyltransferase (LFNG) promoter, a Kringle Containing Transmembrane Protein 1 (KREMEN1) promoter, an Anterior Gradient 3, Protein Disulphide Isomerase Family Member (AGR3) promoter, a SRY-Box 9 (SOX9), or a Solute Carrier Family 6 Member 14 (SLC6A14) promoter.
E116. The nucleic acid vector of E113, wherein the pol II promoter is a ubiquitous promoter.
E117. The nucleic acid vector of E116, wherein the ubiquitous pol II promoter is a CMV promoter, a CAG promoter, or a smCBA promoter.
E117. The nucleic acid vector of E113, wherein the pol II promoter is a hair cell promoter.
E119. The nucleic acid vector of E118, wherein the hair cell promoter is a Myosin 15A (MYO15) promoter, a Growth Factor Independent 1 Transcriptional Repressor (GFI1) promoter, a POU Class 4 Homeobox 3 (POU4F3) promoter, or Myosin 7a (MYO7A) promoter.
E120. The nucleic acid vector of E113, wherein the pol II promoter is a Type II vestibular hair cell promoter.
E121. The nucleic acid vector of E120, wherein the Type II vestibular hair cell promoter is a Calbindin 2 (CALB2) promoter, a Microtubule associated protein tau (MAPT) promoter, an Annexin A4 (ANXA4) promoter, or an Otoferlin (OTOF) promoter.
E122. The nucleic acid vector of any one of E77-E112, wherein the promoter is a pol III promoter.
E123. The nucleic acid vector of E122, wherein the pol III promoter is a ubiquitous pol III promoter.
E124. The nucleic acid vector of E123, wherein the ubiquitous pol III promoter is a U6 promoter, an H1 promoter, or a 7SK promoter.
E125. The nucleic acid vector of any one of E77-124, wherein the nucleic acid vector is a plasmid, cosmid, artificial chromosome, or viral vector.
E126. The nucleic acid vector of E125, wherein the nucleic acid vector is a viral vector.
E127. The nucleic acid vector of E126, wherein the viral vector is selected from the group consisting of an adeno-associated virus (AAV), an adenovirus, and a lentivirus.
E128. The nucleic acid vector of E127, wherein the viral vector is an AAV vector.
E129. The nucleic acid vector of E128, wherein the AAV viral vector has an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ, DJ/8, DJ/9, 7m8, PHP.B, PHP.B2, PBP.B3, PHP.A, PHP.eb, or PHP.S capsid.
E130. An shRNA molecule comprising a nucleotide sequence that has at least 70% complementarity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity) complementarity to any one of SEQ ID NO: 11, SEQ ID NO: 25, SEQ ID NO: 26, SEQ ID NO: 27, SEQ ID NO: 28, and SEQ ID NO: 29.
E131. An shRNA molecule comprising a sequence of nucleotides 2234-2296 of SEQ ID NO: 30 or nucleotides 2234-2296 of SEQ ID NO: 32.
E132. The shRNA molecule of E130 or E131, wherein the shRNA is embedded in a microRNA (miRNA) backbone.
E133. The shRNA molecule of E132, wherein the miRNA backbone is a miR-30 or mir-E backbone.
E134. The shRNA molecule of E133, wherein the shRNA comprises a sequence of nucleotides 2109-2426 of SEQ ID NO: 30, nucleotides 2109-2408 of SEQ ID NO: 31, nucleotides 2109-2426 of SEQ ID NO: 32, or nucleotides 2109-2408 of SEQ ID NO: 33.
E135. A nucleic acid vector comprising a promoter operably linked to the shRNA molecule of any one of E130-E134.
E136. The nucleic acid vector of E135, wherein the vector is an AAV vector.
E137. An siRNA comprising a sense strand and an antisense strand and selected from the following pairs: SEQ ID NO: 35 and SEQ ID NO: 36; SEQ ID NO: 37 and SEQ ID NO: 38; SEQ ID NO: 39 and SEQ ID NO: 40; and SEQ ID NO: 41 and SEQ ID NO: 42.
E138. A nucleic acid vector nucleic acid vector comprising a promoter operably linked to SEQ ID NO: 34, wherein the nucleic acid vector is an AAV vector.

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention. Other embodiments are in the claims.

1. A method of treating a subject having or at risk of developing vestibular dysfunction, comprising administering to the subject an effective amount of a Sox2 inhibitor.

2. The method of claim 1, wherein the Sox2 inhibitor is an inhibitory RNA molecule targeting Sox2 or a Sox2 promoter, a component of a gene editing system targeting Sox2, a polynucleotide encoding a component of a gene editing system targeting Sox2, a dominant negative Sox2 protein, or a polynucleotide encoding a dominant negative Sox2 protein.

3. The method of claim 2, wherein the Sox2 inhibitor is a short interfering RNA (siRNA) or a short hairpin RNA (shRNA).

4. The method of claim 3, wherein the siRNA or shRNA has 100% complementarity to at least 8 contiguous nucleobases of any one of SEQ ID NOs: 5-23 and 25-29.

5. The method of claim 4, wherein the shRNA has 100% complementarity to any one of SEQ ID NO: 11, SEQ ID NO: 25, SEQ ID NO: 26, SEQ ID NO: 27, SEQ ID NO: 28, and SEQ ID NO: 29.

6. The method of claim 5, wherein the shRNA comprises the sequence of nucleotides 2234-2296 of SEQ ID NO: 30 or nucleotides 2234-2296 of SEQ ID NO: 32.

7. The method of any one of claims 3-6, wherein the shRNA is embedded in a microRNA (miRNA) backbone.

8. The method of claim 7, wherein the miRNA backbone is a miR-30 or mir-E backbone.

9. The method of claim 8, wherein the shRNA comprises the sequence of nucleotides 2109-2426 of SEQ ID NO: 30, nucleotides 2109-2408 of SEQ ID NO: 31, nucleotides 2109-2426 of SEQ ID NO: 32, or nucleotides 2109-2408 of SEQ ID NO: 33.

10. The method of claim 4, wherein the siRNA comprises a sense strand and an antisense strand selected from the following pairs: SEQ ID NO: 35 and SEQ ID NO: 36; SEQ ID NO: 37 and SEQ ID NO: 38; SEQ ID NO: 39 and SEQ ID NO:4 0; and SEQ ID NO: 41 and SEQ ID NO: 42.

11. The method of claim 2, wherein the Sox2 inhibitor is a dominant negative Sox2 protein or a polynucleotide encoding a dominant negative Sox2 protein.

12. The method of claim 11, wherein the polynucleotide encoding the dominant negative Sox2 protein has the sequence of SEQ ID NO: 34.

13. The method of any one of claims 1-12, wherein the method further comprises administering a regeneration agent.

14. The method of claim 13, wherein the regeneration agent is an agent that increases Atoh1 expression and/or a Notch inhibitor.

15. The method of claim 14, wherein the regeneration agent is a polynucleotide encoding Atoh1.

16. The method of any one of claims 1-15, wherein the vestibular dysfunction comprises vertigo, dizziness, loss of balance, bilateral vestibulopathy, oscillopsia, or a balance disorder.

17. An shRNA molecule comprising a nucleotide sequence that has 100% complementarity to any one of SEQ ID NO: 11, SEQ ID NO: 25, SEQ ID NO: 26, SEQ ID NO: 27, SEQ ID NO: 28, and SEQ ID NO: 29.

18. An shRNA molecule comprising the sequence of nucleotides 2234-2296 of SEQ ID NO: 30 or nucleotides 2234-2296 of SEQ ID NO: 32.

19. The shRNA molecule of claim 17 or 18, wherein the shRNA is embedded in a microRNA (miRNA) backbone.

20. The shRNA molecule of claim 19, wherein the miRNA backbone is a miR-30 or mir-E backbone.

21. The shRNA molecule of claim 20, wherein the shRNA comprises the sequence of nucleotides 2109-2426 of SEQ ID NO: 30, nucleotides 2109-2408 of SEQ ID NO: 31, nucleotides 2109-2426 of SEQ ID NO: 32, or nucleotides 2109-2408 of SEQ ID NO: 33.

22. A nucleic acid vector comprising a promoter operably linked to the shRNA molecule of any one of claims 17-21.

23. The nucleic acid vector of claim 22, wherein the vector is an AAV vector.

24. An siRNA comprising a sense strand and an antisense strand selected from the following pairs: SEQ ID NO: 35 and SEQ ID NO:36; SEQ ID NO: 37 and SEQ ID NO: 38; SEQ ID NO: 39 and SEQ ID NO: 40; and SEQ ID NO: 41 and SEQ ID NO: 42.

25. A nucleic acid vector nucleic acid vector comprising a promoter operably linked to SEQ ID NO: 34, wherein the nucleic acid vector is an AAV vector.


SEQUENCE LISTING (46 sequences)

SEQ ID NO: 1 - DNA, 2512 bp, Homo sapiens
gatggttgtctattaacttgttcaaaaaagtatcaggagttgtcaaggcagagaagagagtgtttgcaaaagggggaaagtagtttgctgcctctttaagactaggactgagagaaagaagaggagagagaaagaaagggagagaagtttgagccccaggcttaagcctttccaaaaaataataataacaatcatcggcggcggcaggatcggccagaggaggagggaagcgctttttttgatcctgattccagtttgcctctctctttttttcccccaaattattcttcgcctgattttcctcgcggagccctgcgctcccgacacccccgcccgcctcccctcctcctctccccccgcccgcgggccccccaaagtcccggccgggccgagggtcggcggccgccggcgggccgggcccgcgcacagcgcccgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgagggccggacagcgaactggaggggggagaaattttcaaagaaaaacgagggaaatgggaggggtgcaaaagaggagagtaagaaacagcatggagaaaacccggtacgctcaaaaagaaaaaggaaaaaaaaaaatcccatcacccacagcaaatgacagctgcaaaagagaacaccaatcccatccacactcacgcaaaaaccgcgatgccgacaagaaaacttttatgagagagatcctggacttctttttgggggactatttttgtacagagaaaacctggggagggtggggagggcgggggaatggaccttgtatagatctggaggaaagaaagctacgaaaaactttttaaaagttctagtggtacggtaggagctttgcaggaagtttgcaaaagtctttaccaataatatttagagctagtctccaagcgacgaaaaaaatgttttaatatttgcaagcaacttttgtacagtatttatcgagataaacatggcaatcaaaatgtccattgtttataagctgagaatttgccaatatttttcaaggagaggcttcttgctgaattttgattctgcagctgaaatttaggacagttgcaaacgtgaaaagaagaaaattattcaaatttggacattttaattgtttaaaaattgtacaaaaggaaaaaattagaataagtactggcgaaccatctctgtggtcttgtttaaaaagggcaaaagttttagactgtactaaattttataacttactgttaaaagcaaaaatggccatgcaggttgacaccgttggtaatttataatagcttttgttcgatcccaactttccattttgttcagataaaaaaaaccatgaaattactgtgtttgaaatattttcttatggtttgtaatatttctgtaaatttattgtgatattttaaggttttcccccctttattttccgtagttgtattttaaaagattcggctctgtattatttgaatcagtctgccgagaatccatgtatatatttgaactaatatcatccttataacaggtacattttcaacttaagtttttactccattatgcacagtttgagataaataaatttttgaaatatggacactgaaa

SEQ ID NO: 2 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 3 - DNA, 2480 bp, Mus musculus
ggatggttgtctattaacttgttcaaaaaagtatcaggagttgtcaaggcagagaagagagtgtttgcaaaaagggaaaagtactttgctgcctctttaagactagggctgggagaaagaagaggagagagaaagaaaggagagaagtttggagcccgaggcttaagcctttccaaaaactaatcacaacaatcgcggcggcccgaggaggagagcgcctgttttttcatcccaattgcacttcgcccgtctcgagctccgcttccccccaactattctccgccagatctccgcgcagggccgtgcacgccgaggcccccgcccgcggcccctgcatcccggcccccgagcgcggcccccacagtcccggccgggccgagggttggcggccgccggcgggccgcgcccgcccagcgcccgcatgtataacatgatggagacggagctgaagccgccgggcccgcagcaagcttcggggggcggcggcggaggaggcaacgccacggcggcggcgaccggcggcaaccagaagaacagcccggaccgcgtcaagaggcccatgaacgccttcatggtatggtcccgggggcagcggcgtaagatggcccaggagaaccccaagatgcacaactcggagatcagcaagcgcctgggcgcggagtggaaacttttgtccgagaccgagaagcggccgttcatcgacgaggccaagcggctgcgcgctctgcacatgaaggagcacccggattataaataccggccgcggcggaaaaccaagacgctcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcaaccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagctccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcacatgtgagggctggactgcgaactggagaaggggagagattttcaaagagatacaagggaattgggaggggtgcaaaaagaggagagtaggaaaaatctgataatgctcaaaaggaaaaaaaatctccgcagcgaaacgacagctgcggaaaaaaaccaccaatcccatccaaattaacgcaaaaaccgtgatgccgactagaaaacttttatgagagatcttgggacttctttttgggggactatttttgtacagagaaaacctgagggcggcggggagggcgggggaatcggaccatgtatagatctggaggaaaaaaactacgcaaaacttttttttaaagttctagtggtacgttaggcgcttcgcagggagttcgcaaaagtctttaccagtaatatttagagctagactccgggcgatgaaaaaaaagttttaatatttgcaagcaacttttgtacagtatttatcgagataaacatggcaatcaaatgtccattgtttataagctgagaatttgccaatatttttcgaggaaagggttcttgctgggttttgattctgcagcttaaatttaggaccgttacaaacaaggaaggagtttattcggatttgaacattttagttttaaaattgtacaaaaggaaaacatgagagcaagtactggcaagaccgttttcgtggtcttgtttaaggcaaacgttctagattgtactaaatttttaacttactgttaaaggcaaaaaaaaaatgtccatgcaggttgatatcgttggtaatttataatagcttttgttcaatcctaccctttcattttgttcacataaaaaatatggaattactgtgtttgaaatattttcttatggtttgtaatatttctgtaaattgtgatattttaaggtttttccccccttttattttccgtagttgtattttaaaagattcggctctgttattggaatcaggctgccgagaatccatgtatatatttgaactaataccatccttataacagctacattttcaacttaagtttttactccattatgcacagtttgagataaataaatttttgaaatatggacactgaaaaaaaaaaaaaaaa

SEQ ID NO: 4 - protein, 319 aa, Mus musculus
MYNMMETELKPPGPQQASGGGGGGGNATAAATGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGGGLNQRMDSYAHMNGWSNGSYSMMQEQLGYPQHPGLNAHGAAQMQPMHRYVVSALQYNSMTSSQTYMNGSPTYSMSYSQQGTPGMALGSMGSVVKSEASSSPPVVTSSSHSRAPCQAGDLRDMISMYLPGAEVPEPAAPSRLHMAQHYQSGPVPGTAKYGTLPLSHM

SEQ ID NO: 5 - DNA, 21 bp, Artificial Sequence
  Synthetic Construct
ctgccgagaatccatgtatat

SEQ ID NO: 6 - DNA, 21 bp, Artificial Sequence
  Synthetic Construct
gtacagtatttatcgagataa

SEQ ID NO: 7 - DNA, 21 bp, Artificial Sequence
  Synthetic Construct
aggagcacccggattataaat

SEQ ID NO: 8 - DNA, 21 bp, Artificial Sequence
  Synthetic Construct
tggacagttacgcgcacatga

SEQ ID NO: 9 - DNA, 21 bp, Artificial Sequence
  Synthetic Construct
tcccatcacccacagcaaatg

SEQ ID NO: 10 - DNA, 21 bp, Artificial Sequence
  Synthetic Construct
cgagataaacatggcaatcaa

SEQ ID NO: 11 - DNA, 21 bp, Artificial Sequence
  Synthetic Construct
cgctcatgaagaaggataagt

SEQ ID NO: 12 - DNA, 21 bp, Artificial Sequence
  Synthetic Construct
cagctcgcagacctacatgaa

SEQ ID NO: 13 - DNA, 21 bp, Artificial Sequence
  Synthetic Construct
caacggcagctacagcatgat

SEQ ID NO: 14 - DNA, 21 bp, Artificial Sequence
  Synthetic Construct
ccacctacagcatgtcctact

SEQ ID NO: 15 - DNA, 21 bp, Artificial Sequence
  Synthetic Construct
ccctgcagtacaactccatga

SEQ ID NO: 16 - DNA, 21 bp, Artificial Sequence
  Synthetic Construct
acatgtcccagcactaccaga

SEQ ID NO: 17 - DNA, 21 bp, Artificial Sequence
  Synthetic Construct
gcacatgaacggctggagcaa

SEQ ID NO: 18 - DNA, 21 bp, Artificial Sequence
  Synthetic Construct
gcccacctacagcatgtccta

SEQ ID NO: 19 - DNA, 21 bp, Artificial Sequence
  Synthetic Construct
gaagaaggataagtacacgct

SEQ ID NO: 20 - DNA, 21 bp, Artificial Sequence
  Synthetic Construct
accaatcccatccaaattaac

SEQ ID NO: 21 - DNA, 21 bp, Artificial Sequence
  Synthetic Construct
caaagagatacaagggaattg

SEQ ID NO: 22 - DNA, 21 bp, Artificial Sequence
  Synthetic Construct
tgcgcccagtagactgcacat

SEQ ID NO: 23 - DNA, 21 bp, Artificial Sequence
  Synthetic Construct
cgcggcacagatgcaaccgat

SEQ ID NO: 24 - DNA, 954 bp, Artificial Sequence
  Synthetic Construct
atgtataacatgatggagacggagctgaagccgccgggcccgcagcaagcttcggggggcggcggcggaggaggcaacgccacggcggcggcgaccggcggcaaccagaagaacagcccggaccgcgtcacggggcccatgaacgccttcatggtatggtcccgggggcagctgggtaagatggcccaggagaaccccaagatgcacaactcggagatcagcaagcgcctgggcgcggagtggaaacttttgtccgagaccgagaagcggccgttcatcgacgaggccaagcggctgcgcgctctgcacatgaaggagcacccggattataaataccggccgctggggaaaaccaagacgctcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcaaccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagctccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcac

SEQ ID NO: 25 - DNA, 19 bp, Artificial Sequence
  Synthetic Construct
ccagtaatatttagagcta

SEQ ID NO: 26 - DNA, 19 bp, Artificial Sequence
  Synthetic Construct
ttgtgatattttaaggttt

SEQ ID NO: 27 - DNA, 19 bp, Artificial Sequence
  Synthetic Construct
cttatggtttgtaatattt

SEQ ID NO: 28 - DNA, 22 bp, Artificial Sequence
  Synthetic Construct
ttgattgccatgtttatctcga

SEQ ID NO: 29 - DNA, 22 bp, Artificial Sequence
  Synthetic Construct
ttatctcgataaatactgtaca

SEQ ID NO: 30 - DNA, 6198 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttctcgactagggataacagggtaattgtttgaatgaggcttcagtactttacagaatcgttgcctgcacatcttggaaacacttgctgggattacttcttcaggttaacccaacagaaggctcgagaaggtatattgctgttgacagtgagcgccgagataaacatggcaatcaatagtgaagccacagatgtattgattgccatgtttatctcgatgcctactgcctcgcaattgaaggggctactttaggagcaattatcttgtttactaaaactgaataccttgctatctctttgatacatttttacaaagctgaattaaaatggtataaattaaatcacttttataaattaaatcacttttttacgcgtggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagctcttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 31 - DNA, 6195 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttctcgactagggataacagggtaattgtttgaatgaggcttcagtactttacagaatcgttgcctgcacatcttggaaacacttgctgggattacttcgacttcttaacccaacagaaggctcgagaaggtatattgctgttgacagtgagcgccgagataaacatggcaatcaatagtgaagccacagatgtattgattgccatgtttatctcgatgcctactgcctcggacttcaaggggctagaattcgagcaattatcttgtttactaaaactgaataccttgctatctctttgatacatttttacaaagctgaattaaaatggtataaattaaatcacttttttcaattgacgcgtaattctaccggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagctcttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 32 - DNA, 6198 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttctcgactagggataacagggtaattgtttgaatgaggcttcagtactttacagaatcgttgcctgcacatcttggaaacacttgctgggattacttcttcaggttaacccaacagaaggctcgagaaggtatattgctgttgacagtgagcgcgtacagtatttatcgagataatagtgaagccacagatgtattatctcgataaatactgtacatgcctactgcctcgcaattgaaggggctactttaggagcaattatcttgtttactaaaactgaataccttgctatctctttgatacatttttacaaagctgaattaaaatggtataaattaaatcacttttataaattaaatcacttttttacgcgtggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagctcttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 33 - DNA, 6195 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttctcgactagggataacagggtaattgtttgaatgaggcttcagtactttacagaatcgttgcctgcacatcttggaaacacttgctgggattacttcgacttcttaacccaacagaaggctcgagaaggtatattgctgttgacagtgagcgcgtacagtatttatcgagataatagtgaagccacagatgtattatctcgataaatactgtacatgcctactgcctcggacttcaaggggctagaattcgagcaattatcttgtttactaaaactgaataccttgctatctctttgatacatttttacaaagctgaattaaaatggtataaattaaatcacttttttcaattgacgcgtaattctaccggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagctcttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 34 - DNA, 957 bp, Artificial Sequence
  Synthetic Construct
atgtataacatgatggagacggagctgaagccgccgggcccgcagcaagcttcggggggcggcggcggaggaggcaacgccacggcggcggcgaccggcggcaaccagaagaacagcccggaccgcgtcacggggcccatgaacgccttcatggtatggtcccgggggcagctgggtaagatggcccaggagaaccccaagatgcacaactcggagatcagcaagcgcctgggcgcggagtggaaacttttgtccgagaccgagaagcggccgttcatcgacgaggccaagcggctgcgcgctctgcacatgaaggagcacccggattataaataccggccgctggggaaaaccaagacgctcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcaaccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagctccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcacatg

SEQ ID NO: 35 - RNA, 21 nt, Artificial Sequence
  Synthetic Construct
ccaguaauauuuagagcuauu

SEQ ID NO: 36 - RNA, 21 nt, Artificial Sequence
  Synthetic Construct
uagcucuaaauauuacugguu

SEQ ID NO: 37 - RNA, 21 nt, Artificial Sequence
  Synthetic Construct
cgcucaugaagaaggauaauu

SEQ ID NO: 38 - RNA, 21 nt, Artificial Sequence
  Synthetic Construct
uuauccuucuucaugagcguu

SEQ ID NO: 39 - RNA, 21 nt, Artificial Sequence
  Synthetic Construct
uugugauauuuuaagguuuuu

SEQ ID NO: 40 - RNA, 21 nt, Artificial Sequence
  Synthetic Construct
aaaccuuaaaauaucacaauu

SEQ ID NO: 41 - RNA, 21 nt, Artificial Sequence
  Synthetic Construct
cuuaugguuuguaauauuuuu

SEQ ID NO: 42 - RNA, 21 nt, Artificial Sequence
  Synthetic Construct
aaauauuacaaaccauaaguu

SEQ ID NO: 43 - protein, 354 aa, Homo sapiens
MSRLLHAEEWAEVKELGDHHRQPQPHHLPQPPPPPQPPATLQAREHPVYPPELSLLDSTDPRAWLAPTLQGICTARAAQYLLHSPELGASEAAAPRDEVDGRGELVRRSSGGASSSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPSGGEQPPPPPASCKSDHHHLRTAASYEGGAGNATAAGAQQASGGSQRPTPPGSCRTRFSAPASAGGYSVQLDALHFSTFEDSALTAMMAQKNLSPSLPGSILQPVQEENSKTSPRSHRSDGEFSPHSHYSDSDEAS

SEQ ID NO: 44 - DNA, 1062 bp, Homo sapiens
atgtcccgcctgctgcatgcagaagagtgggctgaagtgaaggagttgggagaccaccatcgccagccccagccgcatcatctcccgcaaccgccgccgccgccgcagccacctgcaactttgcaggcgagagagcatcccgtctacccgcctgagctgtccctcctggacagcaccgacccacgcgcctggctggctcccactttgcagggcatctgcacggcacgcgccgcccagtatttgctacattccccggagctgggtgcctcagaggccgctgcgccccgggacgaggtggacggccggggggagctggtaaggaggagcagcggcggtgccagcagcagcaagagccccgggccggtgaaagtgcgggaacagctgtgcaagctgaaaggcggggtggtggtagacgagctgggctgcagccgccaacgggccccttccagcaaacaggtgaatggggtgcagaagcagagacggctagcagccaacgccagggagcggcgcaggatgcatgggctgaaccacgccttcgaccagctgcgcaatgttatcccgtcgttcaacaacgacaagaagctgtccaaatatgagaccctgcagatggcccaaatctacatcaacgccttgtccgagctgctacaaacgcccagcggaggggaacagccaccgccgcctccagcctcctgcaaaagcgaccaccaccaccttcgcaccgcggcctcctatgaagggggcgcgggcaacgcgaccgcagctggggctcagcaggcttccggagggagccagcggccgaccccgcccgggagttgccggactcgcttctcagccccagcttctgcgggagggtactcggtgcagctggacgctctgcacttctcgactttcgaggacagcgccctgacagcgatgatggcgcaaaagaatttgtctccttctctccccgggagcatcttgcagccagtgcaggaggaaaacagcaaaacttcgcctcggtcccacagaagcgacggggaattttccccccattcccattacagtgactcggatgaggcaagt

SEQ ID NO: 45 - protein, 351 aa, Mus musculus
MSRLLHAEEWAEVKELGDHHRHPQPHHVPPLTPQPPATLQARDLPVYPAELSLLDSTDPRAWLTPTLQGLCTARAAQYLLHSPELGASEAAAPRDEADSQGELVRRSGCGGLSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPNVGEQPPPPTASCKNDHHHLRTASSYEGGAGASAVAGAQPAPGGGPRPTPPGPCRTRFSGPASSGGYSVQLDALHFPAFEDRALTAMMAQKDLSPSLPGGILQPVQEDNSKTSPRSHRSDGEFSPHSHYSDSDEAS

SEQ ID NO: 46 - DNA, 1053 bp, Mus musculus
atgtcccgcctgctgcatgcagaagagtgggctgaggtaaaagagttgggggaccaccatcgccatccccagccgcaccacgtcccgccgctgacgccacagccacctgctaccctgcaggcgagagaccttcccgtctacccggcagaactgtccctcctggatagcaccgacccacgcgcctggctgactcccactttgcagggcctctgcacggcacgcgccgcccagtatctgctgcattctcccgagctgggtgcctccgaggccgcggcgccccgggacgaggctgacagccagggtgagctggtaaggagaagcggctgtggcggcctcagcaagagccccgggcccgtcaaagtacgggaacagctgtgcaagctgaagggtggggttgtagtggacgagcttggctgcagccgccagcgagccccttccagcaaacaggtgaatggggtacagaagcaaaggaggctggcagcaaacgcaagggaacggcgcaggatgcacgggctgaaccacgccttcgaccagctgcgcaacgttatcccgtccttcaacaacgacaagaagctgtccaaatatgagaccctacagatggcccagatctacatcaacgctctgtcggagttgctgcagactcccaatgtcggagagcaaccgccgccgcccacagcttcctgcaaaaatgaccaccatcaccttcgcaccgcctcctcctatgaaggaggtgcgggcgcctctgcggtagctggggctcagccagccccgggagggggcccgagacctaccccgcccgggccttgccggactcgcttctcaggcccagcttcctctgggggttactcggtgcagctggacgctttgcacttcccagccttcgaggacagggccctaacagcgatgatggcacagaaggacctgtcgccttcgctgcccgggggcatcctgcagcctgtacaggaggacaacagcaaaacatctcccagatcccacagaagtgacggagagttttccccccactctcattacagtgactctgatgaggccagt